(19) United States
(12) Patent Application Publication
(10) Pub. No.: US 2025/0041408 A1
(43) Pub. Date: Feb. 6, 2025

(54) SMALL MOLECULE IMMUNOPOTENTIATOR CONJUGATES OF NFKB ACTIVATORS AS ADJUVANTS WITH ENHANCED EFFICACY AND REDUCED TOXICITY

(71) Applicants: Naorem NIHESH, Chicago, IL (US); THE UNIVERSITY OF CHICAGO, Chicago, IL (US)

(72) Inventors: Aaron ESSER-KAHN, Chicago, IL (US); Flora KIMANI, Chicago, IL (US); Saikat MANNA, Chicago, IL (US); Brittany MOSER, Chicago, IL (US); Naorem NIHESH, Chicago, IL (US)

(21) Appl. No.: 18/709,085

(22) PCT Filed: Nov. 10, 2022
(86) PCT No.: PCT/US2022/079647

§ 371 Date: May 10, 2024

Related U.S. Application Data
(60) Provisional application No. 63/343,176, filed on May 18, 2022.
(60) Provisional application No. 63/278,011, filed on Nov. 10, 2021.

(51) Int. Cl. A61K 39/39; A61K 39/00; A61K 39/12; A61K 39/21; A61K 39/215; A61K 47/54; A61P 37/04; C07D 471/04

(52) U.S. Cl. CPC A61K 39/39; A61K 39/0011; A61K 39/12; A61K 39/21; A61K 39/215; A61K 47/545; A61P 37/04; C07D 471/04

(57) ABSTRACT

The present disclosure concerns immunomodulatory compositions and methods of use for enhancing response to an antigen (e.g., in a vaccine), an immunotherapy (e.g., a cancer immunotherapy), or other immune stimulation. The disclosure describes immunomodulators having reduced toxicity and improved immune response compared with existing adjuvants. Further disclosed are methods for improving an immune response to a vaccine antigen, cancer immunotherapeutic, or other immune stimulating agent. The disclosure describes dimeric and polymeric immunomodulators comprising one or more pattern recognition receptor (PRR) agonist moieties and one or more NF-κB inhibitor moieties.

### CROSS-REFERENCE TO RELATED APPLICATIONS

This application claims the benefit of priority of U.S. Provisional Patent Application No. 63/278,011 filed Nov. 10, 2021, and U.S. Provisional Patent Application No. 63/343,176 filed May 18, 2022, which are hereby incorporated by reference in their entirety.

### STATEMENT OF GOVERNMENT SUPPORT

This invention was made with government support under AI112194, AI124286, GM099594, and 75N93019C00041 awarded by the National Institutes of Health. The government has certain rights in the invention.

### BACKGROUND

### 1. Field of the Invention

The invention relates generally to the fields of immunology and immunotherapy. Described herein are methods and compositions that increase the safety and effectiveness of vaccines and immunotherapeutics.

### 2. Background

Toll-like receptor (TLR) activation is linked to the high immunogenicity and protective effects of vaccines.^{1,2} The incorporation of TLR adjuvants in sub-unit and epitope-based vaccine formulations has led to great improvements in both antibody and T-cell levels and antigen specificity.^{3,4} Currently, many small molecule adjuvants have been discovered.^{5,6} However, their tolerability in preclinical and clinical studies have limited the use of many of these compounds requiring either reformulation or redesign.⁷

Historically, discovery of adjuvants has been empirical, but with synthetic small molecule adjuvants, modern drug discovery techniques have been applied to optimize adjuvanticity. This has led to the development of a class of adjuvants collectively referred to as small molecule immune potentiators (SMIPs).^{8,9} In this class, imidazoquinolinones that activate toll-like receptor-7 and toll-like receptor-8 (TLR 7/8) such as imiquimod (R837) and resiquimod (R848) have been extensively studied. Imiquimod is currently approved for clinical immunotherapy use in topical creams.⁹⁻¹¹ These SMIPs have been shown to elicit antigen specific cellular responses when administered as adjuvants.¹²⁻¹⁴ Additionally, activation of TLR7/8 by resiquimod can lead to antitumor activity facilitated by APC activation of CD8⁺ T cells and CD4⁺ Th cells due to to IFN-γ, IL-2, and IL-10 production and hence enhanced proliferation.¹⁵⁻¹⁷ However, the high bioavailability of imidazoquinolinone and structurally-related compounds results in unacceptable levels of systemic inflammation due to adjuvant toxicity, greatly limiting their use.

Accordingly, current vaccination and immunotherapeutic methods, particularly those comprising the use of adjuvants, may pose safety concerns, and there is a need in the art for strategies for increasing the safety and tolerability of vaccines and cancer immunotherapeutics.

### SUMMARY OF THE INVENTION

Described herein are immunomodulators capable of enhancing the immunogenicity and tolerability of protein subunit vaccines. Described herein is a synthetic poly-TLR7/8a-dopamine conjugate, p(TLR7/8a-dopa), which decreases toxicity markers and improves immune response. As described herein, p(TLR7/8a-dopa) generates greater humoral and cellular immunity in the context of antigen vaccination than peptide lacking the immunomodulator. The present disclosure describes and demonstrates a strategy to alter existing responses with immunomodulators, tailoring the activity to the desired level without the need of developing new agonists. The current disclosure also describes the synthesis and use of novel hybrid molecules that include a TLR 7/8 agonist moiety and an NF-κB inhibitor moiety. Small molecule NF-κB inhibitors can reduce systemic toxicity by reducing pro-inflammatory systemic IL-6 and TNF-α levels in vaccine formulations. Conjugating NF-κB inhibiting small molecules to TLR 7/8 agonist adjuvants limits their systemic diffusion and alters the response elicited from immune cells. Safety and tolerability can be improved without affecting adjuvanticity and anti-tumor activity.

The disclosure relates to methods for vaccinating a subject comprising administering a hybrid molecule comprising a TLR 7/8 agonist moiety and an NF-κB inhibitor moiety to the subject. Also described is a method for treatment or prevention of cancer comprising administering a hybrid molecule comprising a TLR 7/8 agonist moiety and an NF-κB inhibitor moiety to a subject. Also provided are pharmaceutical compositions comprising hybrid molecules that include a TLR 7/8 agonist moiety and an NF-κB inhibitor moiety.

Methods include methods for enhancing an immune response in a subject comprising administering an immunomodulator comprising one or more TLR7/8 agonist moieties and one or more NF-κB inhibitor moieties. The TLR7/8 agonist moieties and/or the one or more NF-κB inhibitor moieties may be attached to a polypeptide backbone. An immunomodulator of the disclosure may comprise (a) two or more (e.g., 2, 3, 4, 5, 6, or more) TLR7/8 agonist moieties (e.g., imidazoquinoline or a derivative thereof) attached to a polypeptide backbone; and (b) two or more (e.g., 2, 3, 4, 5, 6, or more) NF-κB inhibitor moieties (e.g., dopamine or a derivative thereof) attached to a polypeptide backbone, each connected by a linker. A schematic overview of such an immunomodulator is provided in FIG. 7. An example immunomodulator of the present disclosure is provided at FIG. 8B and FIG. 9.

Described herein are compounds, molecules, polymers, polypeptides, immunomodulators, monomeric immunomodulators, polymeric immunomodulators, PRR agonists, TLR agonists, TLR7/8 agonists, NF-κB inhibitors, methods for synthesis of immunomodulators, methods for immune activation, methods for TLR activation, methods for immune modulation, methods for NF-κB inhibition, pharmaceutical compositions, vaccination methods, methods for enhancing an immune response to an antigen, methods for treatment of cancer, and methods for prevention of cancer.

Compounds of the present disclosure include polymers, polypeptides, immunomodulators (including monomeric and polymeric immunomodulators), TLR agonists (including TLR7/8 agonists), and NF-κB inhibitors. A compound of the present disclosure can comprise at least 1, 2, 3, or more of: a TLR agonist moiety (e.g., a TLR7/8 agonist moiety), an NK-kB inhibitor moiety, a linker, an amino acid, and a polypeptide backbone. Any one or more of these components may be excluded from a compound of the disclosure.

Methods of the present disclosure include treatment methods, disease prevention methods, vaccination methods, synthesis methods, immune activation methods, and cellular activation methods. A method of the present disclosure can include at least 1, 2, 3, or more of the following steps: administering an immunomodulator, administering a polymeric immunomodulator, administering p(TLR7/8a_dopa), administering a vaccine, administering an antigen, generating a pharmaceutical composition comprising an antigen and an immunomodulator, diagnosing a subject as having cancer, diagnosing a subject as having a viral infection, diagnosing a subject as having an autoimmune condition, and administering a cancer therapy. Any one or more of these steps may be excluded from a method of the disclosure.

Pharmaceutical compositions of the present disclosure include compositions for vaccination, compositions for disease treatment, compositions for disease prevention, compositions for immune activation, and compositions for immune suppression. A pharmaceutical composition of the disclosure may comprise at least 1, 2, 3, or more of: an immunomodulator, a polymeric immunomodulator, p(TLR7/8a_dopa), an NF-κB inhibitor, a TLR agonist, a TLR7/8 agonist, an antigen, a bacterial antigen, a viral antigen, a tumor antigen, a peptide, an excipient, a carrier, and a salt.

Provided herein is a compound of formula (I)

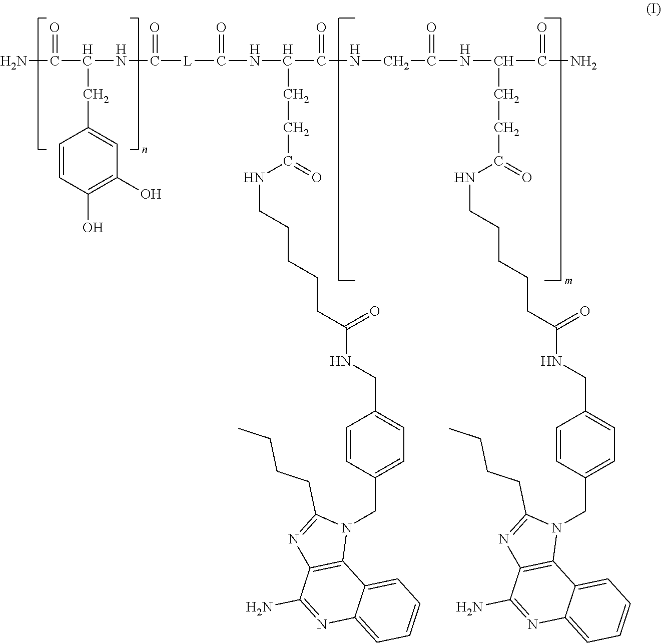

wherein L is a linker; and
wherein n is an integer from 0 to 10 and m is an integer from 0 to 10.

The linker may be a bond, —NH—, —CH₂—, —C(O)—, —C(O)N—, -alkyl-C(O)—, -alkenyl-C(O)—, -alkyl-NC(O)—, -alkenyl-NC(O)—, —C(O)NC(O)—, —C(O)NH(CH₂)_{z}NC(O)—, -alkyl-C(O)NH(CH₂)_{z}NC(O)—, or -alkenyl-C(O)NH(CH₂)_{z}NC(O)—, where z is an integer from 1 to 10.

The linker may be a DBCO linker. The linker may comprise

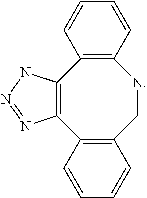

The compound may be further defined as

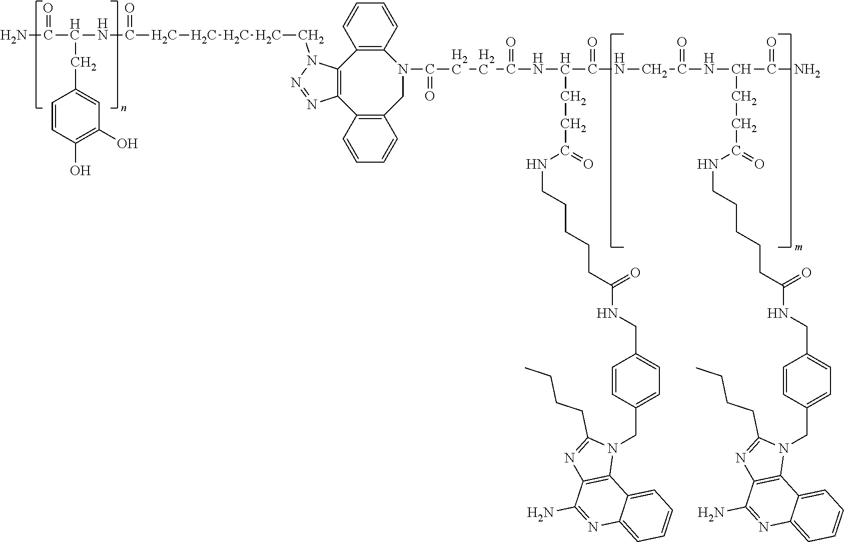

wherein n is an integer from 0 to 10 and m is an integer from 0 to 10.

n can be 0, 1, 2, 3, 4, 5, 6, 7, 8, 9, or 10. m can be 0, 1, 2, 3, 4, 5, 6, 7, 8, 9, or 10. n may be 1 when m is 0, 1, 2, 3, 4, 5, 6, 7, 8, 9, or 10. n may be 2 when m is 0, 1, 2, 3, 4, 5, 6, 7, 8, 9, or 10. n may be 3 when m is 0, 1, 2, 3, 4, 5, 6, 7, 8, 9, or 10. n may be 4 when m is 0, 1, 2, 3, 4, 5, 6, 7, 8, 9, or 10. n is 5 when m is 0, 1, 2, 3, 4, 5, 6, 7, 8, 9, or 10. n may be 6 when m is 0, 1, 2, 3, 4, 5, 6, 7, 8, 9, or 10. n may be 7 when m is 0, 1, 2, 3, 4, 5, 6, 7, 8, 9, or 10. n may be 8 when m is 0, 1, 2, 3, 4, 5, 6, 7, 8, 9, or 10. n may be 9 when m is 0, 1, 2, 3, 4, 5, 6, 7, 8, 9, or 10. n may be 10 when m is 0, 1, 2, 3, 4, 5, 6, 7, 8, 9, or 10. m may be 1 when n is 0, 1, 2, 3, 4, 5, 6, 7, 8, 9, or 10. m may be 2 when n is 0, 1, 2, 3, 4, 5, 6, 7, 8, 9, or 10. m may be 3 when n is 0, 1, 2, 3, 4, 5, 6, 7, 8, 9, or 10. m may be 4 when n is 0, 1, 2, 3, 4, 5, 6, 7, 8, 9, or 10. m may be 5 when n is 0, 1, 2, 3, 4, 5, 6, 7, 8, 9, or 10. m may be 6 when n is 0, 1, 2, 3, 4, 5, 6, 7, 8, 9, or 10. m may be 7 when n is 0, 1, 2, 3, 4, 5, 6, 7, 8, 9, or 10. m may be 8 when n is 0, 1, 2, 3, 4, 5, 6, 7, 8, 9, or 10. m may be 9 when n is 0, 1, 2, 3, 4, 5, 6, 7, 8, 9, or 10. m may be 10 when n is 0, 1, 2, 3, 4, 5, 6, 7, 8, 9, or 10. n may be 5 and m is 4.

The compound may be further defined as

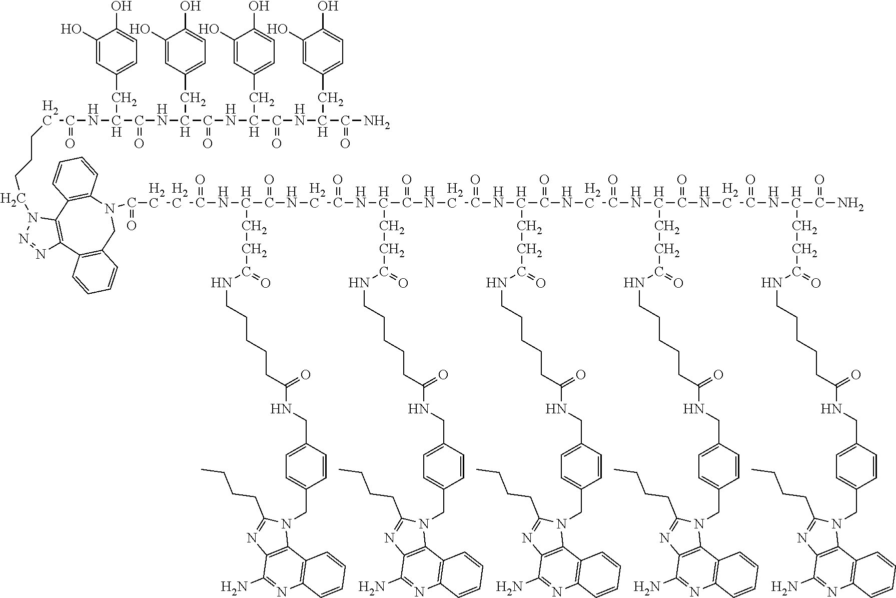

Also disclosed herein is a compound of formula (II)

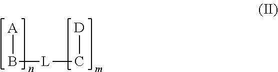

wherein:
A is an NF-κB inhibitor moiety;
B is an amino acid or amino acid analog;
L is a linker;
C is an amino acid or amino acid analog;
D is a TLR agonist moiety; and
wherein n is an integer from 1 to 10 and m is an integer from 1 to 10.

n may be 1, 2, 3, 4, 5, 6, 7, 8, 9, or 10. m may be 1, 2, 3, 4, 5, 6, 7, 8, 9, or 10. The linker may be a bond, —NH—, —CH₂—, —C(O)—, —C(O)N—, -alkyl-C(O)—, -alkenyl-C(O)—, -alkyl-NC(O)—, -alkenyl-NC(O)—, —C(O)NC(O)—, —C(O)NH(CH₂)_{z}NC(O)—, -alkyl-C(O)NH(CH₂)_{z}NC(O)—, or -alkenyl-C(O)NH(CH₂)_{z}NC(O)—, where z is an integer from 1 to 10. The linker may be a DBCO linker. The linker may comprise a region of formula

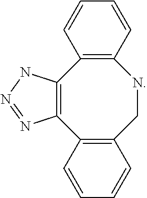

The NF-κB inhibitor moiety may be or may comprise a NF-κB inhibitor of Table 1 or a derivative or portion thereof having NF-κB inhibitor activity. The NF-κB inhibitor moiety may be ferrulic acid or a derivative thereof. The NF-κB inhibitor moiety may be vanillin or a derivative thereof. The NF-κB inhibitor moiety may be honokiol or a derivative. The NF-κB inhibitor moiety may be dopamine or a derivative thereof.

B may be an amino acid of Table 2 or an analog thereof. B may be an amino acid of Table 3 or an analog thereof. B may be glutamic acid or an analog thereof. C may be an amino acid of Table 2 or an analog thereof. C may be an amino acid of Table 3 or an analog thereof. C may be glycine or an analog thereof.

The compound may be further defined as:

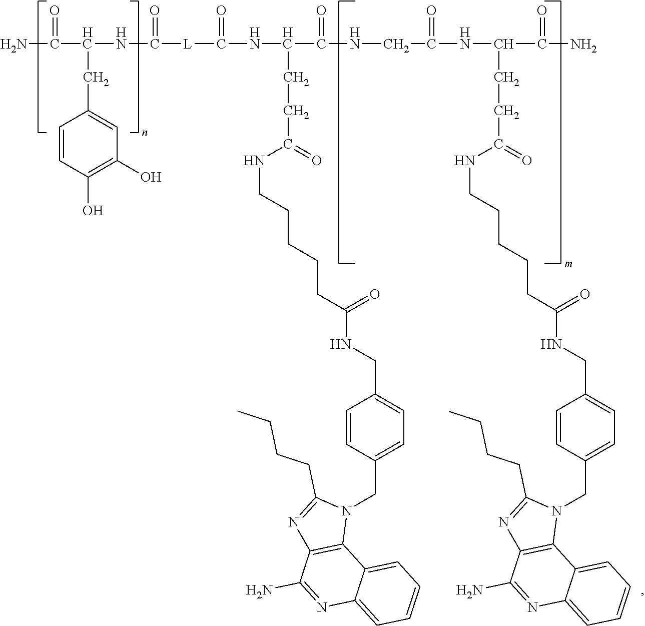

wherein n is an integer from 0 to 10 and m is an integer from 0 to 10.

The compound may be further defined as:

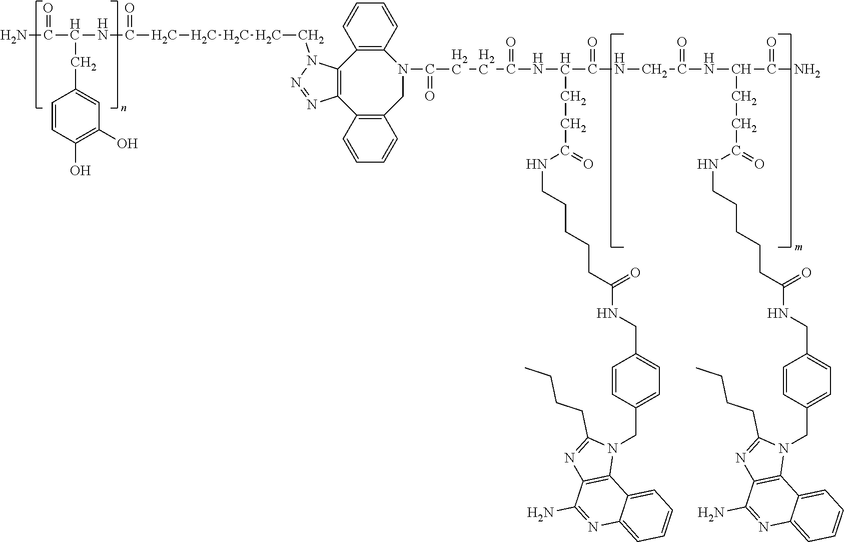

wherein n is an integer from 0 to 10 and m is an integer from 0 to 10.

n may be 0, 1, 2, 3, 4, 5, 6, 7, 8, 9, or 10. m may be 0, 1, 2, 3, 4, 5, 6, 7, 8, 9, or 10. n may be 1 when m is 0, 1, 2, 3, 4, 5, 6, 7, 8, 9, or 10. n may be 2 when m is 0, 1, 2, 3, 4, 5, 6, 7, 8, 9, or 10. n may be 3 when m is 0, 1, 2, 3, 4, 5, 6, 7, 8, 9, or 10. n may be 4 when m is 0, 1, 2, 3, 4, 5, 6, 7, 8, 9, or 10. n may be 5 when m is 0, 1, 2, 3, 4, 5, 6, 7, 8, 9, or 10. n may be 6 when m is 0, 1, 2, 3, 4, 5, 6, 7, 8, 9, or 10. n may be 7 when m is 0, 1, 2, 3, 4, 5, 6, 7, 8, 9, or 10. n may be 8 when m is 0, 1, 2, 3, 4, 5, 6, 7, 8, 9, or 10. n may be 9 when m is 0, 1, 2, 3, 4, 5, 6, 7, 8, 9, or 10. n may be 10 when m is 0, 1, 2, 3, 4, 5, 6, 7, 8, 9, or 10. m may be 1 when n is 0, 1, 2, 3, 4, 5, 6, 7, 8, 9, or 10. m may be 2 when n is 0, 1, 2, 3, 4, 5, 6, 7, 8, 9, or 10. m may be 3 when n is 0, 1, 2, 3, 4, 5, 6, 7, 8, 9, or 10. m may be 4 when n is 0, 1, 2, 3, 4, 5, 6, 7, 8, 9, or 10. m may be 5 when n is 0, 1, 2, 3, 4, 5, 6, 7, 8, 9, or 10. m may be 6 when n is 0, 1, 2, 3, 4, 5, 6, 7, 8, 9, or 10. m may be 7 when n is 0, 1, 2, 3, 4, 5, 6, 7, 8, 9, or 10. m may be 8 when n is 0, 1, 2, 3, 4, 5, 6, 7, 8, 9, or 10. m may be 9 when n is 0, 1, 2, 3, 4, 5, 6, 7, 8, 9, or 10. m may be 10 when n is 0, 1, 2, 3, 4, 5, 6, 7, 8, 9, or 10. n may be 5 and m is 4.

The compound may be further defined as:

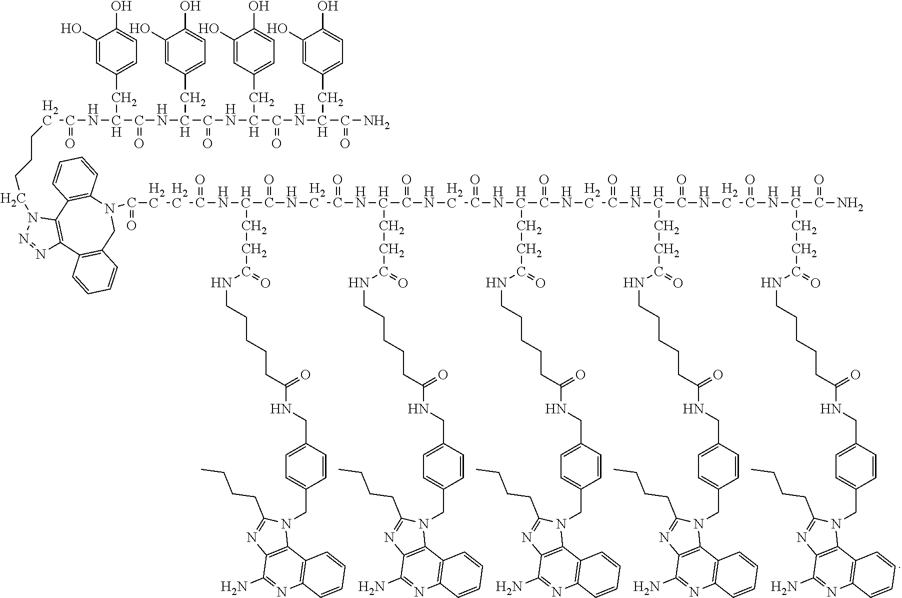

Also disclosed is a method of immune activation comprising administering to a population of immune cells a compound (e.g., polymeric immunomodulator) disclosed herein. The population of immune cells may comprise T cells. The population of immune cells may comprise macrophages. The method may be an in vitro method. The method may be an in vivo method.

Further disclosed herein is a method for vaccinating a subject comprising administering to the subject an effective amount of a pharmaceutical composition comprising a compound (e.g., polymeric immunomodulator) disclosed herein. The method may comprise or further comprise administering an antigen to the subject. The pharmaceutical composition may comprise or further comprise the antigen. The method may be for preventing a disease in the subject. The method may be for treating a disease in the subject. The subject may be one that has previously been administered an adjuvant. The subject may be one that has had an adverse reaction to the adjuvant.

Also disclosed herein is a method for treatment or prevention of cancer, the method comprising administering to a subject an effective amount of a pharmaceutical composition comprising a compound (e.g., polymeric immunomodulator) disclosed herein. The method may comprise or further comprise administering to the subject an additional cancer therapy. The additional cancer therapy may comprise chemotherapy, radiation therapy, immunotherapy, or a combination thereof. The additional cancer therapy may comprise immunotherapy, for example administration of a checkpoint inhibitor. The subject may be one that has not been diagnosed with cancer. The subject may be one that has been diagnosed with cancer. The subject may be one that was previously treated for cancer with a previous therapy. The subject may be determined to be resistant to the previous therapy. The pharmaceutical composition may be administered intratumorally.

Further disclosed is a pharmaceutical composition comprising (a) a compound (e.g., polymeric immunomodulator) disclosed herein and (b) an antigen.

The antigen may be a bacterial antigen. Various bacterial antigens are recognized in the art and contemplated herein. The antigen may be a viral antigen. Various viral antigens are recognized in the art and contemplated herein. Certain examples include, for example, a dengue antigen (e.g., capsid protein of dengue serotype-2 (DENV-2C) or a portion thereof), an HIV antigen (e.g., gp120 or a portion thereof), an influenza antigen, and a SARS-CoV-2 antigen (eg. spike protein or a portion thereof). The antigen may be a tumor antigen. Various tumor antigens are recognized in the art and contemplated herein. A tumor antigen may be an antigen expressed exclusively or preferentially by a tumor cell compared with a healthy cell, including neoantigens discoverable by methods disclosed in the fields of cancer biology and immunology.

The disclosure also describes to a compound of formula (I):

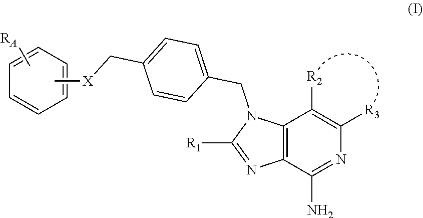

where R₁ is H, C₁-C₁₂ alkyl, C₅-C₁₂ cycloalkyl, C₄-C₁₂ heterocycloalkyl, C₆-C₁₀ aryl, and C₄-C₁₀ heteroaryl, where R₁ is optionally substituted with one or more Y; R₂ and R₃ are independently H, C₁-C₁₂ alkyl, C₂-C₁₂ alkenyl, C₂-C₁₂ alkynyl, C₁-C₁₂ heteroalkyl, C₂-C₁₂ heteroalkenyl, or C₂-C₁₂ heteroalkynyl, or together form a 5-6 membered carbocyclic or heterocyclic ring, where the 5-6 membered carbocyclic or heterocyclic ring is optionally substituted with one or more Y; X is a bond, —N—, —C(O)—, -alkyl-C(O)—, -alkenyl-C(O)—, —C(O)N—, —NC(O)—, -alkyl-NC(O)—, alkenyl-NC(O)—, —C(O)N—, —C(O)NC(O)—, —C(O)NH(CH₂)_{z} NC(O)—, -alkyl-C(O)NH(CH₂)_{z} NC(O)—, or -alkenyl-C(O)NH(CH₂)_{z}NC(O)—; z is an integer from 1 to 10; R_{A} is attached to one or more ring atoms, and each R_{A} is independently hydrogen, hydroxyl, C₁-C₁₂ alkoxy, C₂-C₁₂ alkenoxy, C₂-C₁₂ alkenyl, or substituted or unsubstituted phenyl; and Y is substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NH₂, NO₂, NR₄R₅, NC(O)R₅, OC(O)NR₄R₅, OC(O)OR₄, C(O)R₄, C(O)OR₄, SH, SR₄, OR₄, where R₄ and R₅ are independently C₁-C₆ substituted or unsubstituted alkyl. R₁ may be a C₁-C₁₂ alkyl group. The alkyl group may be an n-butyl group. R₂ and R₃ together can form a phenyl ring. The phenyl ring may be an unsubstituted phenyl ring. X may be an amide. X may comprise one or more of an alkyl group, an alkenyl group, an amide group, and a urea group. R_{A} may be unsubstituted phenyl. R_{A} may be substituted phenyl.

The compound of formula (I) may be further defined as one of:

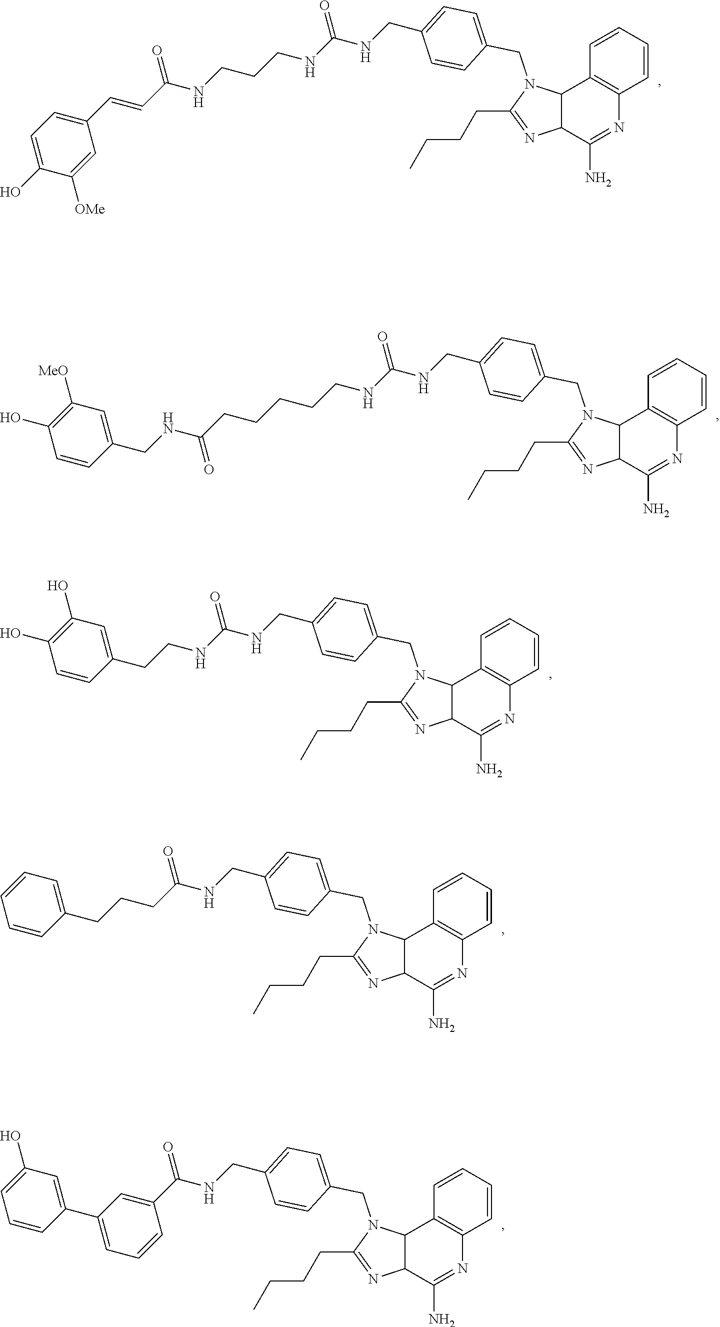 [SMILES: CCCCC1=NC2C(N)=Nc3ccccc3C2N1Cc1ccc(CNC(=O)NCCCNC(=O)/C=C/c2ccc(O)c(OC)c2)cc1 ; CCCCC1=NC2C(N)=Nc3ccccc3C2N1Cc1ccc(CNC(=O)NCCCCCC(=O)NCc2ccc(O)c(OC)c2)cc1 ; CCCCC1=NC2C(N)=Nc3ccccc3C2N1Cc1ccc(CNC(=O)NCCc2ccc(O)c(O)c2)cc1 ; CCCCC1=NC2C(N)=Nc3ccccc3C2N1Cc1ccc(CNC(=O)CCCc2ccccc2)cc1 ; CCCCC1=NC2C(N)=Nc3ccccc3C2N1Cc1ccc(CNC(=O)c2cccc(-c3cccc(O)c3)c2)cc1]

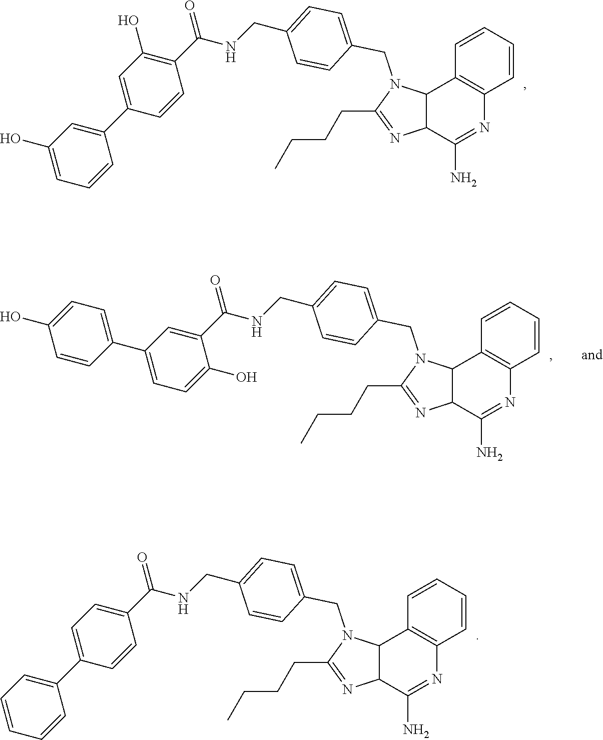 [SMILES: CCCCC1=NC2C(N)=Nc3ccccc3C2N1Cc1ccc(CNC(=O)c2ccc(-c3cccc(O)c3)cc2O)cc1 ; CCCCC1=NC2C(N)=Nc3ccccc3C2N1Cc1ccc(CNC(=O)c2cc(-c3ccc(O)cc3)ccc2O)cc1 ; CCCCC1=NC2C(N)=Nc3ccccc3C2N1Cc1ccc(CNC(=O)c2ccc(-c3ccccc3)cc2)cc1]

Also provided is a compound of formula (II):

A-B-C (II)

where A is a TLR agonist, B is a linker, and C is an NF-κB inhibitor. The TLR agonist may be a TLR 7/8 agonist. The TLR7/8 agonist may be imidazoquinolinone or a derivative thereof. The linker may be a bond, —NH—, —CH₂—, —C(O)—, —C(O)N—, -alkyl-C(O)—, -alkenyl-C(O)—, -alkyl-NC(O)—, -alkenyl-NC(O)—, —C(O)NC(O)—, —C(O)NH(CH₂)_{z}NC(O)—, -alkyl-C(O)NH(CH₂)_{z}NC(O)—, or -alkenyl-C(O)NH(CH₂)_{z}NC(O)—, where z is an integer from 1 to 10. The linker may be oriented in either direction, e.g., an amide linker may be linked to the TLR agonist through the amide amine or through the amide carbon. The NF-κB inhibitor may be ferrulic acid or a derivative thereof. The NF-κB inhibitor may be vanillin or a derivative thereof. The NF-κB inhibitor may be dopamine or a derivative thereof. The NF-κB inhibitor may be honokiol or a derivative thereof. The compound may be further defined as one of:

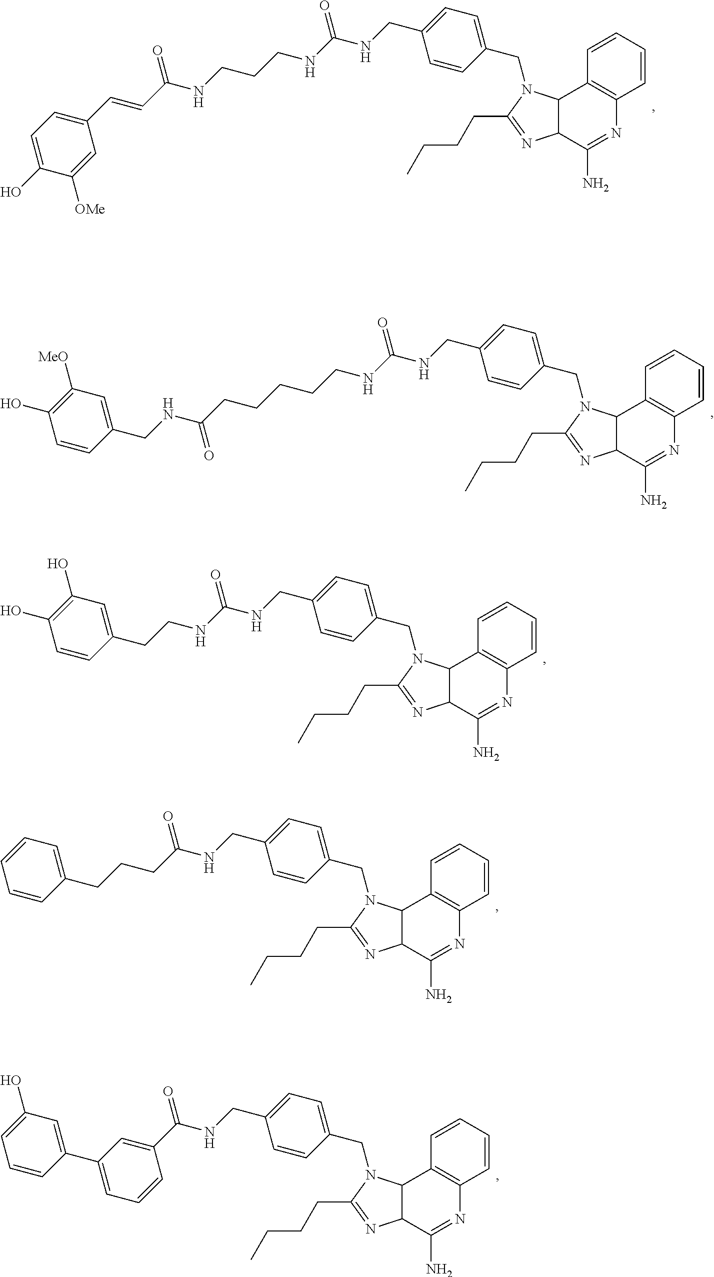 [SMILES: CCCCC1=NC2C(N)=Nc3ccccc3C2N1Cc1ccc(CNC(=O)NCCCNC(=O)/C=C/c2ccc(O)c(OC)c2)cc1 ; CCCCC1=NC2C(N)=Nc3ccccc3C2N1Cc1ccc(CNC(=O)NCCCCCC(=O)NCc2ccc(O)c(OC)c2)cc1 ; CCCCC1=NC2C(N)=Nc3ccccc3C2N1Cc1ccc(CNC(=O)NCCc2ccc(O)c(O)c2)cc1 ; CCCCC1=NC2C(N)=Nc3ccccc3C2N1Cc1ccc(CNC(=O)CCCc2ccccc2)cc1 ; CCCCC1=NC2C(N)=Nc3ccccc3C2N1Cc1ccc(CNC(=O)c2cccc(-c3cccc(O)c3)c2)cc1]

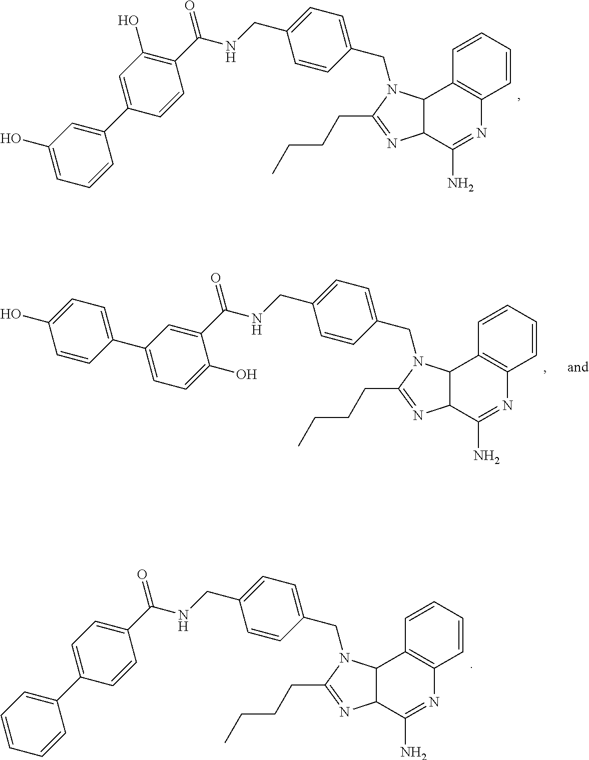 [SMILES: CCCCC1=NC2C(N)=Nc3ccccc3C2N1Cc1ccc(CNC(=O)c2ccc(-c3cccc(O)c3)cc2O)cc1 ; CCCCC1=NC2C(N)=Nc3ccccc3C2N1Cc1ccc(CNC(=O)c2cc(-c3ccc(O)cc3)ccc2O)cc1 ; CCCCC1=NC2C(N)=Nc3ccccc3C2N1Cc1ccc(CNC(=O)c2ccc(-c3ccccc3)cc2)cc1]

Also described is a method for vaccinating a subject. The method may comprise administering to the subject an effective amount of a pharmaceutical composition comprising a compound of formula (I) or a compound of formula (II). The method may further comprise administering an antigen to the subject. The antigen may be a bacterial antigen. The antigen may be a viral antigen. The antigen may be a dengue antigen. The dengue antigen may comprise capsid protein of dengue serotype-2 (DENV-2C). The antigen may be an HIV antigen. The HIV antigen may comprise gp120. The antigen may be a SARS-CoV-2 antigen. The SARS-CoV-2 antigen may be a SARS-CoV-2 spike protein or portion thereof. The antigen may be a tumor antigen The subject may be a human subject. The method may be for preventing a disease in the subject. The method may be for treating a disease in the subject. The subject may be one that has previously been administered an adjuvant. The subject may be one that has had an adverse reaction to the adjuvant.

Also described is a method for treatment or prevention of cancer, the method comprising administering to a subject an effective amount of a pharmaceutical composition comprising a compound of formula (I) or a compound of formula (II). The method may comprise or further comprise administering to the subject an additional cancer therapy. The additional cancer therapy may comprise chemotherapy, radiation therapy, immunotherapy, or a combination thereof. The additional cancer therapy may comprise immunotherapy. The subject may be a human subject. The subject may be a non-human primate, a laboratory animal, a mammal, a rat, dog, pig, horse, mouse, rabbit, goat, or cat. The subject may be one has not been diagnosed with cancer. The subject may be one that has been diagnosed with cancer. The subject may be one that was previously treated for cancer with a previous therapy. The subject may be one that was determined to be resistant to the previous therapy. The pharmaceutical composition may be administered to the subject intratumorally.

Also described are pharmaceutical compositions comprising a compound of formula (I) or a compound of formula (II). The pharmaceutical composition may further comprises an antigen. The antigen may be a bacterial antigen. The antigen may be a viral antigen. The antigen may be a dengue antigen. The dengue antigen may comprise capsid protein of dengue serotype-2 (DENV-2C). The antigen may be an HIV antigen. The HIV antigen may comprise gp120. The antigen may comprise a SARS-CoV-2 antigen. The SARS-CoV-2 antigen may comprise a SARS-CoV-2 spike protein or portion thereof. The antigen may comprise an influenza antigen. The antigen may comprise a tumor antigen.

The compound of formula (I) or formula (II) may be administered by intramucosal, intramuscular, parenteral, or subcutaneous administration. The the compound of formula (I) or formula (II) may be administered by a route of administration described herein. The compound of formula (I) or formula (II) may be administered prior to administration of an antigen. The compound of formula (I) or formula (II) may be administered after administration of an antigen. The compound of formula (I) or formula (II) may be administered at least or at most 0.5, 1, 2, 3, 4, 5, or 10 hours or 1, 2, 3, 4, 5, 6, 7, 8, 9, 10, 11, 12, 13, or 14, days before or after (or any derivable range therein) an antigen. The compound of formula (I) or formula (II) and antigen may be administered simultaneously. A compound of formula (I) or formula (II) and an antigen may be administered locally to the same site in the subject. A compound of formula (I) or formula (II) and an antigen may be administered in the same composition to the subject. The compound of formula (I) or formula (II) may be administered in a separate composition than an antigen. The subject may be one that has had an adverse reaction to a previous administration of an adjuvant or to a vaccine. The adverse reaction may comprise systemic inflammation.

At least 12 mg of a compound of formula (I) or formula (II) may be administered to the subject. At least, at most, or about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 125, 150, 175, 200, 225, 250, 275, 300, 325, 350, 375, 400, 425, 450, 475, or 500 mg of a compound of formula (I) or formula (II) (or any derivable range therein) may be administered to the subject. 0.2 mg/kg a compound of formula (I) or formula (II) may be administered to the subject. At least, at most, or about 0.01, 0.05, 0.07, 0.1, 0.15, 0.2, 0.25, 0.3, 0.35, 0.4, 0.45, 0.5, 0.55, 0.6, 0.65, 0.7, 0.75, 0.8, 0.85, 0.9. 1, 1.5, 2, 2.5, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/kg (or any derivable range therein) of a compound of formula (I) or formula (II) may be administered to the subject. The amount of compound of formula (I) or formula (II) administered to a human or non-human primate subject may correspond to a dose that is equal to or greater than 50 micrograms in a mouse. The amount of compound of formula (I) or formula (II) administered to a human or non-human primate subject may correspond to a dose that is more than or less than 1, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, or 900 micrograms (or any derivable range therein) in a mouse.

The subject may be a human. The subject may be a non-human primate, a mouse, a goat, a rabbit, a dog, a horse, or a sheep.

The method may be for preventing a disease in the subject. The method may be for treating a disease in a subject.

A compound or composition as disclosed herein may be formulated for intramucosal, intramuscular, parenteral, or subcutaneous administration. The composition may further comprise a pharmaceutical excipient.

The methods and compositions of the disclosure may be used to reduce systemic inflammation, such as that associated with vaccination and/or vaccines comprising an adjuvant. The methods of the disclosure may reduce adjuvant-induced inflammation while also increasing the adaptive immune response. The methods of the disclosure may reduce one or both of IL-6 and TNF-α. The methods and compositions of the disclosure may be used to enhance antigen presentation and T cell activation, and/or increase antibody titer. The methods and compositions of the disclosure may also enhance epitope selectivity, shift epitope selectivity, and/or provide for a vaccine that produces a broad-spectrum antibody response.

The preparation of the vaccine as the active immunogenic ingredient, may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

Administration of vaccines according to the disclosure may be via any common route so long as the target tissue is available via that route in order to maximize the delivery of antigen to a site for maximum (or in some cases minimum) immune response. Administration will generally be by orthotopic, intradermal, mucosally, subcutaneous, intramuscular, intraperitoneal or intravenous injection. Other areas for delivery include: oral, nasal, buccal, rectal, vaginal or topical. Vaccines of the invention are preferably administered parenterally, by injection, for example, either subcutaneously or intramuscularly.

Vaccines may be administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, or in the range from about 10 mg to 50 mg. Suitable regimens for initial administration and booster shots are also variable but are typified by an initial administration followed by subsequent inoculations or other administrations. Precise amounts of active ingredient required to be administered depend on the judgment of the practitioner and may be peculiar to each subject. It will be apparent to those of skill in the art that the therapeutically effective amount of nucleic acid molecule or fusion polypeptides of this invention will depend, inter alia, upon the administration schedule, the unit dose of antigen administered, whether the vaccine composition is administered in combination with other therapeutic agents, and the immune status and health of the recipient.

A vaccine may be given in a single dose schedule or in a multiple dose schedule. A multiple dose schedule is one in which a primary course of vaccination may include, e.g., 1-10 separate doses, followed by other doses given at subsequent time intervals required to maintain and/or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity.

A vaccine may be provided in one or more “unit doses”. Unit dose is defined as containing a predetermined-quantity of the vaccine calculated to produce the desired responses in association with its administration, i.e., the appropriate route and treatment regimen. The quantity to be administered, and the particular route and formulation, are within the skill of those in the clinical arts. The subject to be treated may also be evaluated, in particular, the state of the subject's immune system and the protection desired. A unit dose need not be administered as a single injection but may include continuous infusion over a set period of time. Unit dose of the present invention conveniently may be described in terms of mg/kg body weight. The dose of the NFkB inhibitor, adjuvant, or antigen may be at least, at most, or about 0.05, 0.10, 0.15, 0.20, 0.25, 0.5, 1, 10, 50, 100, 1,000 or any derivable range therein mg/kg. Likewise the amount of vaccine delivered can vary from about 0.2 to about 8.0 mg/kg body weight. 0.1 mg/kg, 0.2 mg/kg, 0.3 mg/kg, 0.4 mg/kg, 0.5 mg/kg, 0.8 mg/kg, 1.0 mg/kg, 1.5 mg/kg, 2.0 mg/kg, 2.5 mg/kg, 3.0 mg/kg, 4.0 mg/kg, 5.0 mg/kg, 5.5 mg/kg, 6.0 mg/kg, 6.5 mg/kg, 7.0 mg/kg and 7.5 mg/kg (or any derivable range therein) of the vaccine may be delivered to an individual in vivo. The dosage of vaccine to be administered depends to a great extent on the weight and physical condition of the subject being treated as well as the route of administration and the frequency of treatment.

The methods of the disclosure may comprise administering one or more compositions two or more times. It is contemplated that the compositions may be administered 1, 2, 3, 4, 5, 6, 7, 8, 9, 10, 11, 12, 13 or 14 days apart or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 weeks apart or 1, 2, 3, 4, 5, 6, 7, 8, 9, 10, 11, 12, 13, 14, 15, 16, 17, 18, 19, 20, 21, 22, 23, 24, 36, 48, 60, 72, 84 or 96 months apart or 1, 2, 3, 4, 5, 6, 7, 8, 9, 10, 11, 12, 13, 14, 15, 16, 17, 18, 19 or 20 years apart (or any derivable range therein).

Also described is a kit comprising compositions of the disclosure and instructions for use.

Methods may further comprise testing the patient for an infection, such as a viral infection or diagnosing a patient with an infection, such as a viral infection.

Any embodiment discussed in the context of an antibody may be implemented in any method embodiment discussed herein.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows innate immune activation as measured by NF-κB in RAW-Blue 264.7 macrophage cell assay. Cells were incubated with each compound for 18 h at 37° C. Supernatant was removed and incubated with QUANTIBlue for 1 h, and the absorbance at 620 nm was measured. Error bars represent standard deviation (SD). Samples were run in triplicate. Statistical differences were determined via two-tailed t-test (n=3 per group).

FIGS. 2A-B show immune activation as measured by costimulatory molecules CD40 (FIG. 2A) and CD86 (FIG. 2B) on BMDMs. Cells were incubated with each compound for 18 h at 37° C. Error bars represent SD. Samples were run in triplicate. Statistical differences were determined via two-tailed t-test (n=3 per group). Each experiment was repeated thrice with similar results.

FIG. 3 shows innate immune activation as measured by concentration of TNF-α in the supernatant of BMDMs after 12 h of exposure to TLR7/8 ligand as either pTLR7/8_mod, pTLR7/8, or mTLR7/8. Cells were incubated with each compound for 18 h at 37° C. Supernatant was removed and analyzed using ELISA and the absorbance at 450 nm was measured. Error bars represent SD. Samples were run in triplicate. Statistical differences were determined via two-tailed t-test (n=3 per group).

FIGS. 4A-C show in vivo IL-23 (FIG. 4A), IL6 (FIG. 4B), and MCP-1 (FIG. 4C) serum levels in C57BL/6 mice 24 h postinjection as measured by cytokine cytometric bead array (CBA). C57Bl/6 mice were immunized s.c. with 100 μg of OVA in just alum or with 10 μg of TLR7/8 ligand formulated as p(TLR7/8)+alum, p(TLR7/8_mod)+alum, p(TLR7/8)+pmod+alum, on days 0 and 14. All symbols represent individual mice. Statistical differences were determined via two-tailed t-test (n=5 per group).

FIG. 5 shows serum anti-OVA IgG titers from C57Bl/6 mice (n=5 per group) immunized s.c with 100 μg of OVA in just alum or with 10 μg of TLR7/8 ligand formulated as p(TLR7/8)+alum, p(TLR7/8_mod+alum, p(TLR7/8)+pmod+alum, on days 0 and 14. Serum antibody titers were analyzed by ELISA at 3 weeks. Statistical differences were determined via two-tailed t-test.

FIGS. 6A-C show levels of IL-6 (FIG. 6A), IL-2 (FIG. 6B) and TNF-α (FIG. 6C) analyzed via CBA. C57Bl/6 mice (n=5 per group) were immunized s.c with 100 μg of OVA in alum or with 10 μg of TLR7/8 ligand formulated as p(TLR7/8)+alum, p(TLR7/8_mod+alum, p(TLR7/8)+pmod+alum, on days 0 and 14. Two weeks after the second immunization, cells from the dLNs were collected and re-stimulated with OVA for 48 h and the supernatant analyzed via CBA for cytokines. Statistical differences were determined via two-tailed t-test.

FIG. 7 shows a schematic of a polymeric immunomodulator of the present disclosure. The modulator comprises repeating dopamine units (left; circles) and repeating imidazoquinoline units (right; squares), each attached to a polypeptide backbone and linked via a linker.

FIGS. 8A-B show an overview of the synthesis scheme of a polymeric immunomodulator of the present disclosure—p(TLR7/8a_dopa).

FIG. 9 shows the structure of p(TLR7/8a_dopa).

FIGS. 10A-B (A) Traditional SMIP adjuvant NF-κB pathway. Activation of TLR7/8 with the agonist leads to downstream pro-inflammatory responses and responses associated with adaptive immunity (antigen presentation). (B) SMIP activation of the NF-κB pathway using TLR7/8 agonist/NF-κB inhibitor dimers of the disclosure. Activation of TLR7/8 with the dimers also results in activation of NF-kB pathway by the agonist while the tethered potentiators inhibit specific parts of the pathway resulting in lower pro-inflammatory immune response and enhanced or unchanged adaptive immune response.

FIGS. 11A-C (A) TLR 7/8 small molecule potentiator (SMIP) imidazoquinolinone. (B) NF-κB small molecule inhibitors. (C) Synthesized TLR 7/8 agonist/NF-κB inhibitor dimers 1-6, 12, and 13; intermediate compounds 7-11.

FIGS. 12A-C In vitro assays determining TLR 7/8 NF-κB activation and potentiation of synthesized dimers. (A) Immune activation measured by RAW-Blue activation via NF-κB stimulation after 24 h incubation with 500 nM of compounds at 37° C. (B) IL-6 expression (ELISA). (C) Cell surface protein expression (FACS) measured 8 h after incubation with bone marrow derived macrophages (BMDCs). Compounds assayed at 200 nm.

FIGS. 13A-B (A) Intracellular reactive oxygen species (ROS) measured by incubating RAW macrophages with CM-H2DCFDA (6-chloromethyl-2′,7′-dichlorodihydrofluorescein diacetate, acetyl ester) and fluorescence measured using flow cytometry. (B) Nitrite levels in supernatant of RAW macrophages incubated with 500 nM of compounds for 16 h and measured using Griess reagent.

FIGS. 14A-B (A) Expression of COX-2 protein measured in the lysate of RAW macrophages incubated with 500 nM compounds for 16 h. Samples run in triplicate. (B) Graphical representation of COX-2 expression. Statistical significance to TLR 7/8 SMIP, compared by the one-way ANOVA *p≤0.05, ****p≤0.0001.

FIGS. 15A-C In vivo assays of TLR 7/8 agonist/NF-κB inhibitor dimers. (A) Outline of in vivo vaccination investigation. (B) Serum IL-6 levels assayed 1 h after injection. IL-6 levels of linked compounds not detected. (C) Serum TNF-α levels assayed 1 h after injection. TNF-α levels of linked compounds not detected.

FIGS. 16A-C Serum anti-OVA antibodies measured day 28. (A) Serum anti-OVA Ig. (B) Serum anti-OVA IgG. (C) Serum anti-OVA IgA. Statistical significance to PBS control, compared by the one-way ANOVA *p≤0.05, ****p≤0.0001.

FIGS. 17A-C (A) Outline of in vivo tumor model experiment. (B) Systemic IL-6 levels measured in the serum 2 h after intertumoral injection of compounds. (C) Systemic TNF-α levels measured in the serum 2 h after intertumoral injection of compounds.

FIGS. 18A-C (A) Hematological analysis of peripheral blood white blood cells (WBC) measured 24 h after intertumoral injection of compounds. (B) Hematological analysis of peripheral blood lymphocytes measured 24 h after intertumoral injection of compounds. (C) Survivability plot. Statistical significance to PBS, compared by the one-way ANOVA *p≤0.05, ****p≤0.0001.

FIGS. 19A-B In vivo assays with R848 and NF-κB inhibitor. Serum levels of cytokines assayed 1 h after injection. (A) IL-6 levels are not significantly reduced when compared with R848. (B) TNF-α levels are not significantly reduced when compared with R848.

FIG. 20 MTT assay showing the viability of agonist and agonist dimer treated cells. At the assayed concentrations the cells have comparable viability to resting cells.

FIGS. 21A-B (A) SINFEKL MHC-specific tetramer on day 28 post-vaccination isolated spleens. (B) Proliferation assay on naïve spleenocytes.

FIG. 22 Overlay of results of six in vivo tumor model analyses using SMIP-modulator dimers with peritumoral injection into subcutaneous CT-26 tumor model. Agonists were injected when tumors were about 75 cc in size followed by three additional injections every four days.

FIGS. 23A-F In vivo tumor model analyses using SMIP-modulator dimers with peritumoral injection into subcutaneous CT-26 tumor model. (A) PBS control. (B) TLR 7/8 SMIP. (C) TLR 7/8 agonist resiquimod (R-848). (D) TLR 7/8 agonist 3M-052. (E) Compound 1. (F) Compound 2.

### DESCRIPTION OF ILLUSTRATIVE EMBODIMENTS

Imidazoquinolinone derivatives that activate Toll like receptor (TLR) 7/8 are small molecule immune potentiators (SMIPs) that have potent activity as vaccine adjuvants and as anti-tumor agents. However, these molecules have high bioavailability that results in unacceptable levels of systemic inflammation due to adjuvant toxicity greatly limiting their use.

Small molecule NF-κB inhibitors can be used to potentiate cytidine phosphate guanosine (CPG, a TLR 9 agonist) in vaccine formulations. The small molecule NF-κB inhibitors capsaicin and honokiol were shown to reduce pro-inflammatory systemic IL-6 and TNF-α levels while maintaining vaccine protective effects.¹⁹ However, this effect was not observed when the in-vivo experiments were repeated with R848 as an adjuvant. This is likely due to the high diffusion of the small molecule adjuvant and the immune potentiators.

The present disclosure is based, at least in part, on the design of hybrid molecules in which an imidazoquinolinone derivative²⁰ was covalently linked through an conjugatable amine handle to vanilloid, catechol and honokiol¹⁹ derivatives in order to reduce the degree of diffusion. Using in vitro assays, a mini library of synthesized dimers was screened and viable candidates were selected for further in vivo experiments. Mice were vaccinated with ovalbumin as a model antigen treated with the synthesized dimers. The results demonstrated that these dimers reduce systemic toxicity to baseline levels while maintaining the adjuvanticity in a vaccine formulation. Additionally, select dimers increased survivability in a CT26 WT mouse colon carcinoma tumor model while eliciting low adjuvant toxicity.

### I. DEFINITIONS

The term “adjuvant” as used herein refers to substances, which when administered prior, together or after administration of an antigen, accelerate, prolong and/or enhance the quality and/or strength of an immune response to the antigen in comparison to the administration of the antigen alone.

NFκB (also “NF-κB” or “NFkB”) refers to the protein nuclear factor kappa B (encoded by the gene NFKB1).

As used herein, the term “vaccine” describes a composition which can be administered to humans or to animals in order to induce an immune system response; this immune system response can result in a production of antibodies or simply in the activation of certain cells, for example antigen-presenting cells, T lymphocytes and/or B lymphocytes. The vaccine may be capable of producing an immune response that leads to the production of neutralizing antibodies in the patient with respect to the antigen provided in the vaccine. The vaccine can be a composition for prophylactic purposes or for therapeutic purposes, or both.

As used herein, the term “antigen” refers to any antigen that can be used in a vaccine, whether it involves a whole microorganism or a portion thereof, and various types: (e.g., peptide, protein, glycoprotein, polysaccharide, glycolipid, lipopeptide, etc). Thus, the term “antigen” refers to a molecule that can initiate a humoral and/or cellular immune response in a recipient of the antigen. The antigen may be a molecule that causes a disease for which a vaccination would be advantageous treatment. The antigen may comprise a substance used to stimulate the production of antibodies and provide immunity against one or several diseases, prepared from the causative agent of a disease, its products, or a synthetic substitute, treated to act as an antigen without inducing the disease. The antigen may comprise a peptide or polypeptide.

As used herein, the term “dimer” refers to a bi-functional molecule. A dimer of the disclosure can include a TLR receptor-binding moiety and a NF-κB-binding moiety. The terms “dimer,” “hybrid molecule,” “adjuvant dimer,” “dimer adjuvant,” “dimer agonist,” and “agonist dimer” are used interchangeably herein.

The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the vaccine can contain minor amounts of auxiliary substances such as wetting or emulsifying agents, and pH buffering agents.

As used herein, the term “agonist” refers to a molecule that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another molecule so that the other molecule directly binds to the receptor. An agonist may be referred to as an agonist of a particular receptor or family of receptors (e.g., a TLR agonist).

“Individual, “subject,” and “patient” are used interchangeably and can refer to a human or non-human.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C6-C10-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄C₁, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplated. In certain embodiments, an aryl group may be mono-, di-, tri-, tetra- or penta-substituted with one or more heteroatom-containing substituents.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cₙ-alkoxy, and heteroatom-substituted Cₙ-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “alkenyl” includes straight and branched chain hydrocarbon radicals containing one double bond and having from 2 to 6 carbon atoms such as, for example, ethenyl, 2-propenyl (allyl), 3-butenyl, 2-pentenyl, 3-pentenyl, 3-methyl-2-butenyl, and the like. The term “alkenoxy” refers to an alkenyl ether radical, where alkenyl is defined as above.

The term “allyl” refers to the radical H₂C═CH—CH₂. The term “ether” refers to a hydrocarbyl group that is attached to another hydrocarbyl group via oxygen. Thus, the ether substituent of the hydrocarbyl group can be hydrocarbyl-O—. The ether can be symmetric or asymmetric. Examples of ethers include, but are not limited to vinyl ether and allyl ether. The term “vinyl”—refers to the portion of a molecule that includes a carbon-carbon double bond.

Various groups described herein, including hydroxyl, aryl, alkenoxy, alkoxy, and alkenyl, may be optionally substituted with one or more substituents. Non-limiting examples of substituent groups include halogen, nitro, cyano, hydroxy, amino, mercapto, formyl, carboxy, carbamoyl, alkyl, heteroalkyl, alkoxy, alkylthio, alkylamino, (alkyl)2amino, alkylsulfinyl, alkylsulfonyl, arylsulfonyl, cycloalkyl, heterocyclyl, aryl, and heteroaryl.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term consisting essentially of may include the listed active ingredients, such as the recited dimer, and also any unrecited buffers, pharmaceutical excipients, etc. but exclude any other active ingredients, such as other hybrid molecules.

### II. IMMUNOMODULATORS

Methods and compositions of the disclosure are directed to immunomodulators and methods of use thereof. As used herein, an “immunomodulator” describes any molecule capable of stimulating an innate immune response in a subject. Immunomodulators include molecules capable of activating a patent recognition receptor (PRR), such as a toll-like receptor (TLR), in an immune cell. The disclosure includes polymeric immunomodulators comprising multiple TLR agonist moieties and multiple NF-κB inhibitor moieties. As disclosed herein, such a polymeric molecule is capable of activating a TLR while limiting release of proinflammatory cytokines, thus improving safety and tolerability without affecting adjuvanticity and/or anti-tumor activity. The number of TLR agonist moieties and NF-κB inhibitor moieties on a particular polymeric immunomodulator can be adjusted to tune the desired level of immune activation.

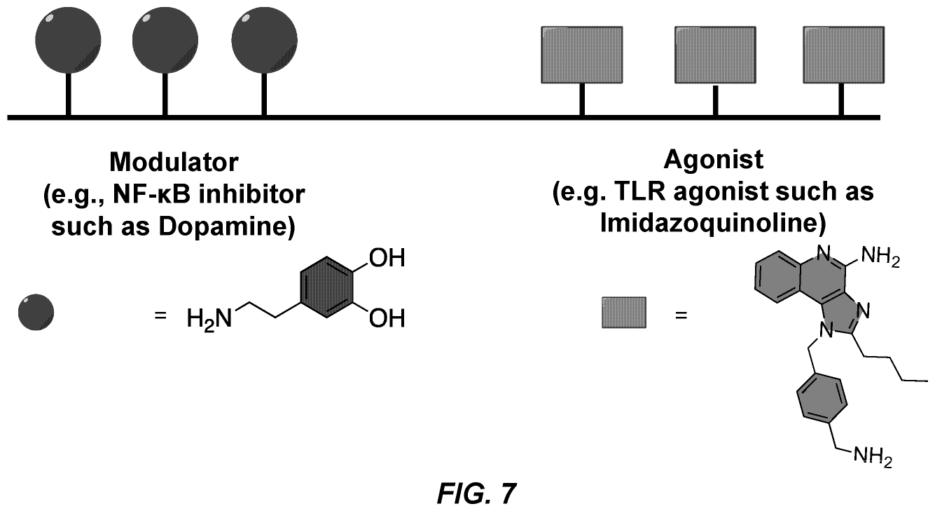

Disclosed are polymeric immunomodulators built on a polypeptide backbone comprising two or more repeating TLR agonist moieties and two or more repeating NF-κB inhibitor moieties. A schematic example of such a polymeric immunomodulator is provided in FIG. 7. A polymeric immunomodulator of the disclosure may comprise multiple different TLR agonist moieties and/or repeating units of the same TLR agonist moiety. A “TLR agonist moiety” describes a moiety of a molecule or compound having TLR agonist activity. A polymeric immunomodulator of the disclosure may comprise multiple different NF-κB inhibitor moieties and/or repeating units of the same NF-κB inhibitor moiety. An “NF-κB inhibitor moiety” describes a moiety of a molecule or compound having NF-κB inhibitor activity.

A polymeric immunomodulator may comprise a first region comprising two or more TLR agonist moieties attached to a polypeptide backbone and a second region comprising two or more NF-κB inhibitor moieties attached to a polypeptide backbone. The polypeptide backbone of the first region may comprise all of the same amino acid subunits (e.g., repeating glutamic acid units), or multiple different amino acid subunits. The polypeptide backbone of the second region may comprise all of the same amino acid subunits (e.g., repeating glycine units), or multiple different amino acid subunits. The first region may comprise at least, at most, or exactly 2, 3, 4, 5, 6, 7, 8, 9, 10, 11, 12, 13, 14, 15, 16, 17, 18, 19, or 20 TLR agonist moieties. The first region may comprise exactly 2, 3, 4, 5, 6, 7, or 8 TLR agonist moieties. The second region may comprise at least, at most, or exactly 2, 3, 4, 5, 6, 7, 8, 9, 10, 11, 12, 13, 14, 15, 16, 17, 18, 19, or 20 NF-κB inhibitor moieties. The second region may comprise exactly 2, 3, 4, 5, 6, 7, or 8 NF-κB inhibitor moieties. A polymeric immunomodulator of the present disclosure may comprise the same number of TLR agonist moieties as NF-κB inhibitor moieties, or may comprise different numbers of TLR agonist moieties and NF-κB inhibitor moieties. For example, the immunomodulator may comprise 3 TLR agonist moieties and 3 NF-κB inhibitor moieties, or the immunomodulator may comprise 3 TLR agonist moieties and 5 NF-κB inhibitor moieties.

The immunomodulator may comprise 3 TLR agonist moieties and 2, 3, 4, 5, 6, 7, or 8 NF-κB inhibitor moieties. The immunomodulator may comprise 4 TLR agonist moieties and 2, 3, 4, 5, 6, 7, or 8 NF-κB inhibitor moieties. The immunomodulator may comprise 5 TLR agonist moieties and 2, 3, 4, 5, 6, 7, or 8 NF-κB inhibitor moieties. The immunomodulator may comprise 6 TLR agonist moieties and 2, 3, 4, 5, 6, 7, or 8 NF-κB inhibitor moieties. The immunomodulator may comprise 7 TLR agonist moieties and 2, 3, 4, 5, 6, 7, or 8 NF-κB inhibitor moieties. The immunomodulator may comprise 8 TLR agonist moieties and 2, 3, 4, 5, 6, 7, or 8 NF-κB inhibitor moieties.

The first region and the second region may be connected by a linker. Various linkers are recognized in the art and contemplated herein. The first and second region may be connected by a triazole linker.

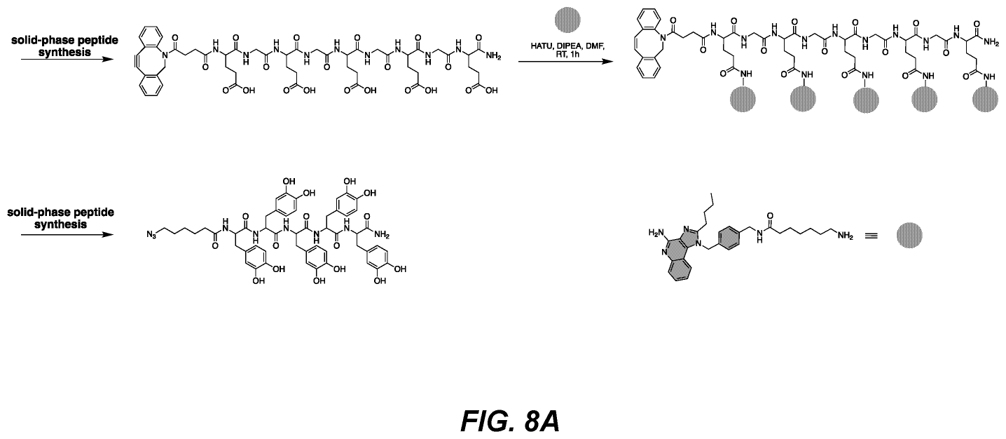

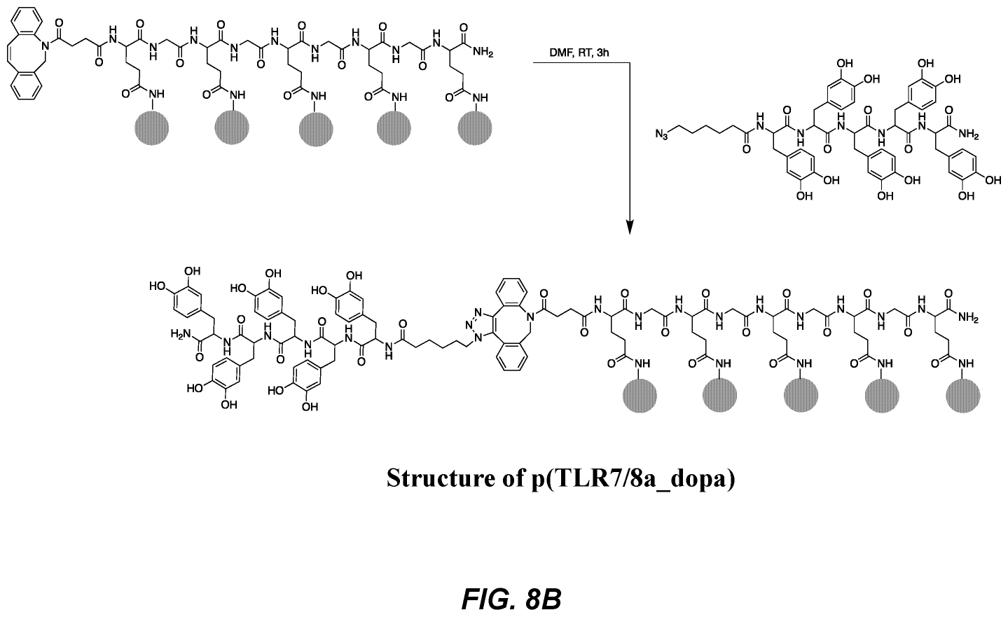

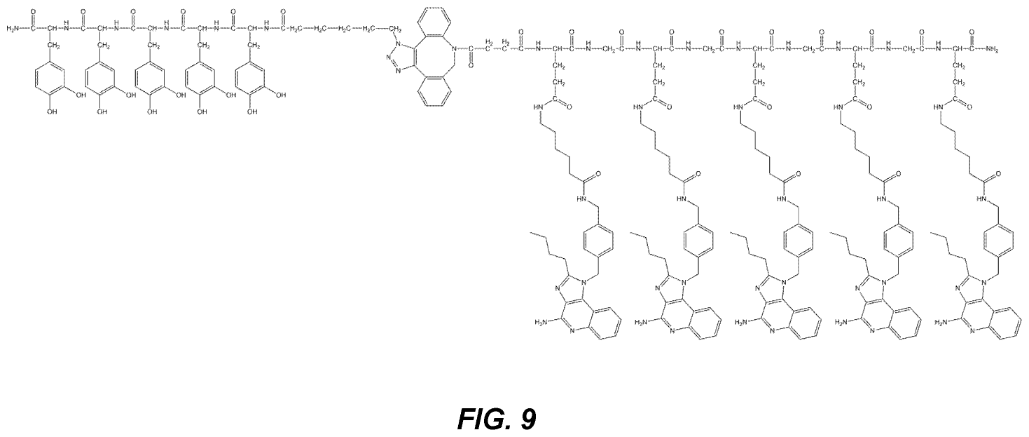

A polymeric immunomodulator may comprise a polypeptide backbone, where each of the TLR agonist moieties and/or NF-κB inhibitor moieties are attached to the side chain of each amino acid of the polypeptide backbone. For example, a polymeric immunomodulator of the disclosure comprises a region of poly-glutamic acid, where each glutamic acid residue is attached to a TLR agonist moiety (e.g., TLR 7/8 agonist such as imidazoquinoline) via the side chain. A non-limiting example of such a polymeric immunomodulator is provided at FIG. 8B and FIG. 9.

Disclosed is an immunomodulator having formula (I):

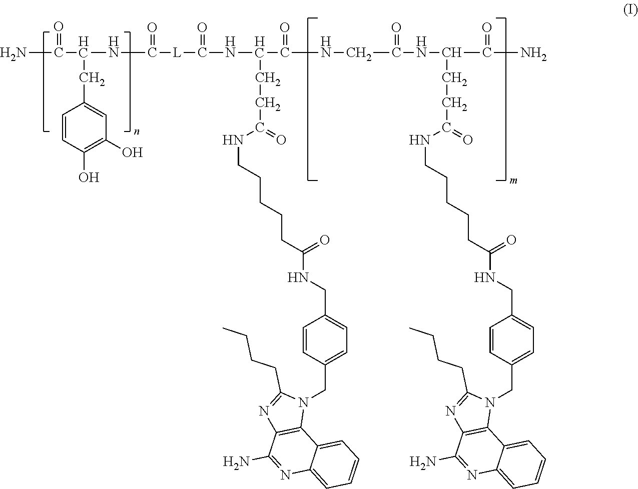

wherein L is a linker; and
wherein n is an integer from 1 to 10 and m is an integer from 1 to 10.

n may be at least, at most, or exactly 1, 2, 3, 4, 5, 6, 7, 8, 9, or 10. n may be at least 2. n may be 5. m may be at least, at most, or exactly 1, 2, 3, 4, 5, 6, 7, 8, 9, or 10. m may be at least 2. m ma be 5. n may be equal to m. n may be not equal to m. The immunomodulator may be further defined as:

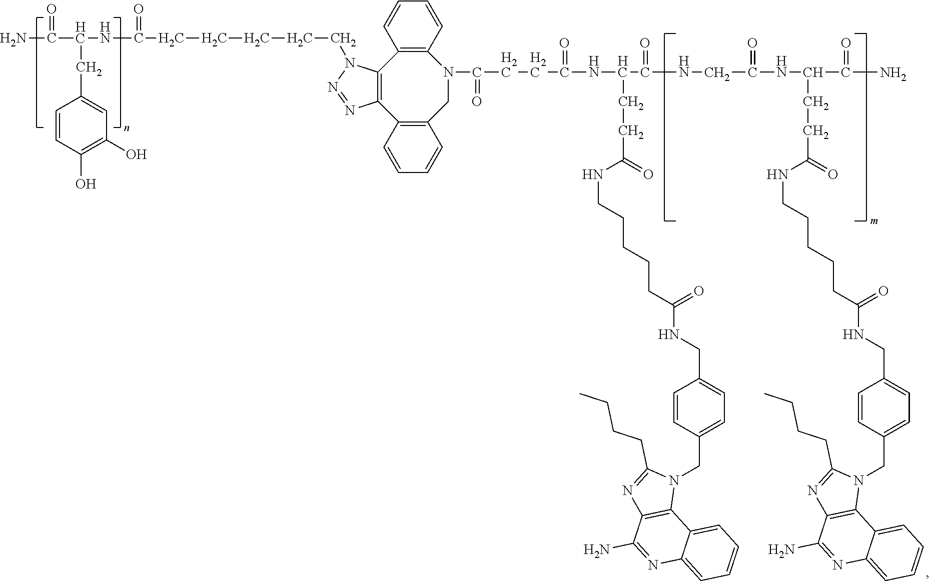

wherein n is an integer from 1 to 10 and m is an integer from 1 to 10. n and m may both be 5.

The immunomodulator may be further defined as:

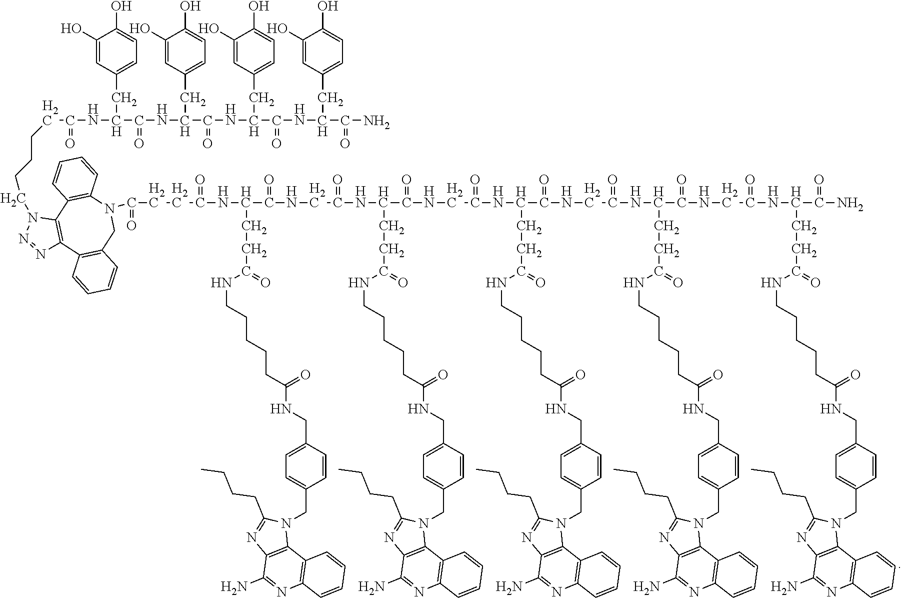

Disclosed is an immunomodulator having formula (II):

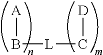

wherein:
A is an NF-κB inhibitor moiety;
B is an amino acid;
L is a linker;
C is an amino acid; and
D is a TLR agonist moiety,
   wherein n is an integer from 1 to 10 and m is an integer from 1 to 10.

The TLR agonist may be a TLR1, TLR2, TLR3, TLR4, TLR5, TLR6, TLR7, TLR8, and or TLR9 agonist. The TLR agonist may be a TLR7/8 agonist, such as imidazoquinolinone or a derivative thereof having TLR7/8 agonist activity. The NF-κB inhibitor may be ferrulic acid or a derivative thereof having NF-κB inhibitor activity. The NF-κB inhibitor may be dopamine or a derivative thereof having NF-κB inhibitor activity. The NF-κB inhibitor may be vanillin or a derivative thereof having NF-κB inhibitor activity. The NF-κB inhibitor may be honokiol or a derivative thereof having NF-κB inhibitor activity.

B and C may be the same amino acid. B and C may be different amino acids. B may be an amino acid of Table 2. B may be an amino acid of Table 3. B may be a glutamic acid residue attached to the NF-κB inhibitor moiety. C may be an amino acid of Table 2. C may be an amino acid of Table 3. C may be a glutamic acid residue attached to the TLR agonist moiety.

n may be at least, at most, or exactly 1, 2, 3, 4, 5, 6, 7, 8, 9, or 10. n may be at least 2. n may be 5. m may be at least, at most, or exactly 1, 2, 3, 4, 5, 6, 7, 8, 9, or 10. m may be at least 2. m may be 4. n may be equal to m. n may be unequal to m. The immunomodulator is further defined as:

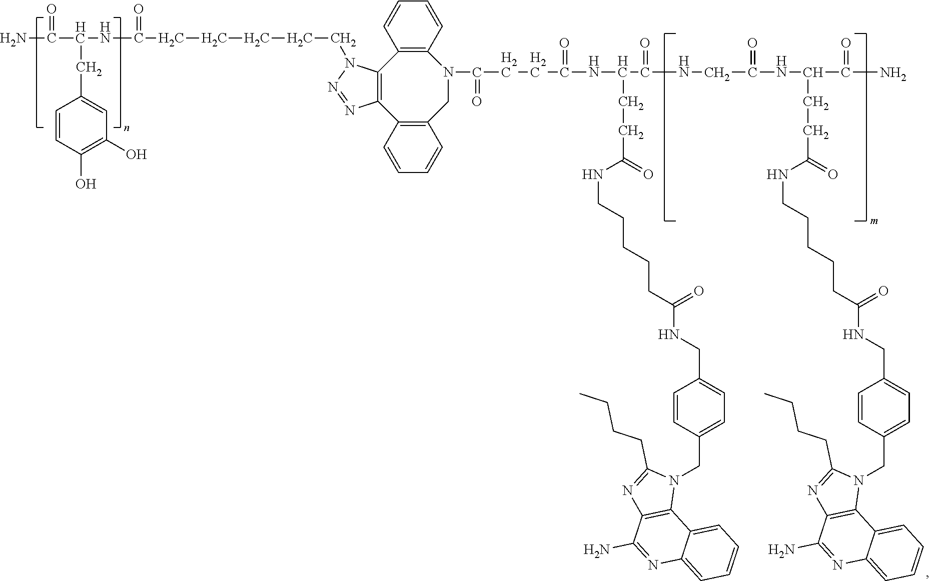

wherein n is an integer from 1 to 10 and m is an integer from 1 to 10. n is 5 and m may be 5.

The immunomodulator may be further defined as:

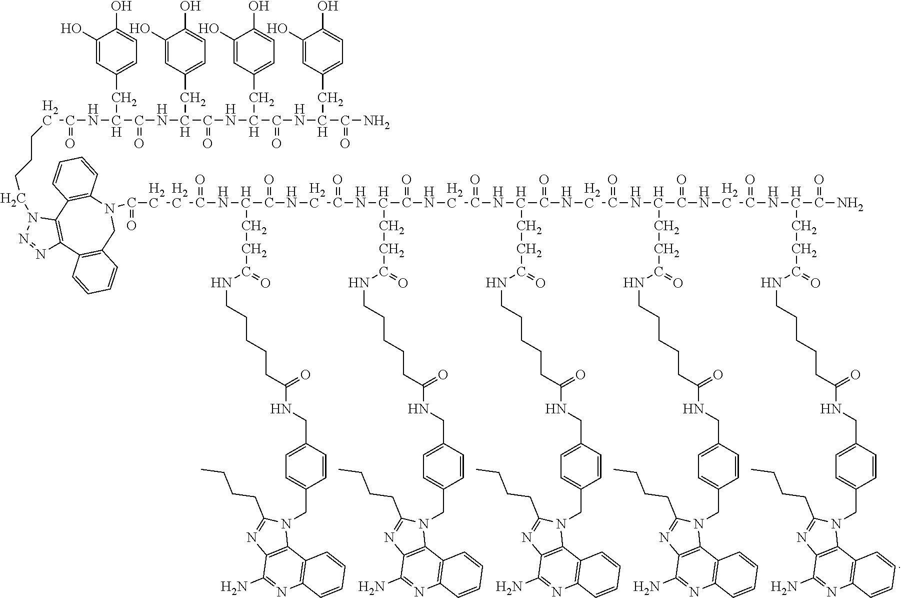

### III. NF-κB INHIBITORS

The disclosure includes compositions and methods comprising or directed to one or more NF-κB inhibitors, or derivatives thereof. As used herein, an NF-κB inhibitor describes any molecule capable of inhibiting expression or activity of NF-κB, which activity includes activating or promoting transcription of one or more genes. “NF-κB potentiator,” as used herein, describes an NF-κB inhibitor, or derivative thereof, which is capable of potentiating the activity of a TLR agonist. Table 1 provides example NF-κB inhibitors useful in the methods and compositions of the disclosure.

TABLE 1

| Molecule | Type of molecule | Point of inhibition |
|---|---|---|
| Calagualine (fern derivative) | Natural product | Upstream of IKK (TRAF2-NIK) |
| Conophylline (Ervatamia microphylla) | Natural product | Down regulated TNF-Receptors |
| Evodiamine (Evodiae fructus component) | Natural product | AKT-IKK interaction |
| Geldanamycin | Natural product | IKK complex formation |
| Perrilyl alcohol | Natural product | Calcium pathway |
| Protein-bound polysaccharide from basidiomycetes | Natural product | LPS-CD 14 interaction |
| Rocaglamides (Aglaia derivatives) | Natural product | Upstream of IKK |
| 15-deoxy-prostaglandin J(2) | Natural product | PPARγ activation of NF-κB |
| Adenovirus E1A | Protein, viral | Adaptor |
| NS5A (Hepatitis C virus) | Protein, viral | TRAF2 inhibition |
| NS3/4A (HCV protease) | Protein | Upstream of IKK |
| Golli BG21 (product of myelin basic protein) | Protein | Upstream of IKK (PKC) |
| NPM-ALK oncoprotein | Protein | Traf2 inhibition |
| MAST205 | Protein | TRAF6 binding |
| Erbin overexpression | Protein | NOD2 inhibitor |
| Rituximab (anti-CD20 antibody) | Protein | Up-regulates Raf-1 kinase inhibitor |
| Kinase suppressor of ras (KSR2) | Protein | MEKK3 inhibitor |
| PEDF (pigment epithelium derived factor) | Protein | ROS generation |
| TNAP | Protein | NIK |
| Betaine | Synthetic | NIK/IKK |
| Desloratadine | Synthetic | Histamine H1 receptor |
| LY29 and LY30 | Synthetic | PI3 Kinase inhibitors |
| MOL 294 (small molecule) | Synthetic | Redox regulated activation of NF-κB |
| Pefabloc (serine protease inhibitor) | Synthetic | Upstream of IKK |
| Rhein | Synthetic | MEKK activation of NF-κB |
| Salmeterol, fluticasone propionate | Synthetic | β2 agonists |
| Lead | Inorganic Complex | IKK activity |
| Anandamide | Natural product | IKKβ activity |
| Artemisia vestita | Natural product | Phosphorylation |
| Cobrotoxin | Natural product | IKKβ activity and p50 DNA binding |
| Dehydroascorbic acid (Vitamin C) | Natural product | IKKβ activity |
| Herbimycin A | Natural product | IKKβ activity |
| Isorhapontigenin | Natural product | IKKβ activity |
| Manumycin A | Natural product | IKKβ activity |
| Pomegranate fruit extract | Natural product | IKKα activity |
| Tetrandine (plant alkaloid) | Natural product | IKKα activity |
| Nitric oxide | Natural product | IKKβ activity/IκB phosphorylation |
| Thienopyridine | Natural product | IKKβ activity |
| Acetyl-boswellic acids | Natural product | IKK activity |
| β-carboline | Natural product | IKK activity |
| 1'-Acetoxychavicol acetate (Languas galanga) | Natural product | IKK activity |
| Apigenin (plant flavinoid) | Natural product | IKK activity |
| Cardamomin | Natural product | IKK activity |
| Diosgenin | Natural product | IKK activity |
| Furonaphthoquinone | Natural product | IKK activity |
| Guggulsterone | Natural product | IKK activity |
| Falcarindol | Natural product | IKK activity |
| Honokiol | Natural product | IKK activity |
| Hypoestoxide | Natural product | IKK activity |
| Garcinone B | Natural product | IKK activity |
| Kahweol | Natural product | IKK activity |
| Kava (Piper methysticum) derivatives | Natural product | IKK activity |
| γ-mangostin (from Garcinia mangostana) | Natural product | IKK activity |
| N-acetylcysteine | Natural product | IKK activity |
| Nitrosylcobalamin (vitamin B12 analog) | Natural product | IKK activity |
| Piceatannol | Natural product | IKK activity |
| Plumbagin (5-hydroxy-2-methyl-1,4-naphthoquinone) | Natural product | IKK activity |
| Quercetin | Natural product | IKK activity |
| Rosmarinic acid | Natural product | IKK activity |
| Semecarpus anacardiu extract | Natural product | IKK activity |
| Staurosporine | Natural product | IKK activity |
| Sulforaphane and phenylisothiocyanate | Natural product | IKK activity |
| Theaflavin (black tea component) | Natural product | Activation of IKK |
| Tilianin | Natural product | Activation of IKK |
| γ-Tocotrienol | Natural product | IKK activity |
| Wedelolactone | Natural product | IKK activity |
| Withanolides | Natural product | Activation of IKK |
| Zerumbone | Natural product | Activation of IKK |
| Silibinin | Natural product | IKKα activity; nuclear translocation |
| Betulinic acid | Natural product | IKKα activity and p65 phosphorylation |
| Ursolic acid | Natural product | IKKα activity and p65 phosphorylation |
| Monochloramine and glycine chloramine (NH2Cl) | Natural product | Oxidizes IκB |
| Anethole | Natural product | Phosphorylation |
| Baoganning | Natural product | Phosphorylation |
| Black raspberry extracts (cyanidin 3-O-glucoside, | Natural product | Phosphorylation |
| cyanidin 3-O-(2(G)-xylosylrutinoside), | | |
| cyanidin 3-O-rutinoside) | | |
| Buddlejasaponin IV | Natural product | Phosphorylation |
| Cacospongionolide B | Natural product | Phosphorylation |
| Calagualine | Natural product | Phosphorylation |
| Carbon monoxide | Natural product | Phosphorylation |
| Cardamonin | Natural product | Phosphorylation |
| Cycloepoxydon; 1-hydroxy-2-hydroxymethyl-3- | Natural product | Phosphorylation |
| pent-1-enylbenzene | | |
| Decursin | Natural product | Phosphorylation |
| Dexanabinol | Natural product | Phosphorylation |
| Digitoxin | Natural product | Phosphorylation |
| Diterpenes | Natural product | Phosphorylation |
| Docosahexaenoic acid | Natural product | Phosphorylation |
| Extensively oxidized low densityl ipoprotein | Natural product | Phosphorylation |
| (ox-LDL), 4-Hydroxynonenal (HNE) | | |
| Flavopiridol | Natural product | IKK activity and RelA phosphory lation |
| [6]-gingerol; casparol | Natural product | Phosphorylation |
| Glossogyne tenuifolia | Natural product | Phosphorylation |
| Guggulsterone | Natural product | Phosphorylation |
| Indirubin-3'-oxime | Natural product | Phosphorylation |
| Licorce extracts | Natural product | Phosphorylation |
| Oleandrin | Natural product | Phosphorylation |
| Omega 3 fatty acids | Natural product | Phosphorylation |
| Panduratin A (from Kaempferia pandurata, | Natural product | Phosphorylation |
| Zingiberaceae) | | |
| Petrosaspongiolide M | Natural product | Phosphorylation |
| Pinosylvin | Natural product | Phosphorylation |
| Plagius flosculosus extract polyacetylene spiroketal | Natural product | Phosphorylation |
| Phytic acid (inositol hexakisphosphate) | Natural product | Phosphorylation |
| Pomegranate fruit extract | Natural product | Phosphorylation |
| Prostaglandin A1 | Natural product | Phosphorylation/IKK |
| 20(S)-Protopanaxatriol (ginsenoside metabolite) | Natural product | Phosphorylation |
| Rengyolone | Natural product | Phosphorylation |
| Rottlerin | Natural product | Phosphorylation |
| Saikosaponin-d | Natural product | Phosphorylation |
| Saline (low Na+ istonic) | Natural product | Phosphorylation |
| Salvia miltiorrhizae water-soluble extract | Natural product | Phosphorylation |
| Sanguinarine (pseudochelerythrine, 13-methyl-[1,3]- | Natural product | Phosphorylation |
| benzodioxolo-[5,6-c]-1,3-dioxolo-4,5 phenanthridinium) | | |
| Sesquiterpene lactones (parthenolide; ergolide; guaianolides) | Natural product | IKK and DNA binding |
| Scoparone | Natural product | Phosphorylation |
| Silymarin | Natural product | Phosphorylation |
| Sulindac | Natural product | IKK/Phosphorylation |
| Vesnarinone | Natural product | Phosphorylation |
| Xanthoangelol D | Natural product | Phosphorylation |
| IKKß peptide to NEMO binding domain | Peptide | IKK-NEMO interaction |
| NEMO CC2-LZ peptide | Peptide | NEMO oligomerization |
| Adenovirus E3-14.7K | Protein, viral | IKK activity |
| Adenovirus E3-10.4/14.5K | Protein, viral | IKK activity |
| Core protein (Hepatitis C virus) | Protein, viral | IKKβ activity |
| E7 (Papillomavirus) | Protein, viral | IKK activity |
| MC160 (Mollusum Contagiosum virus) | Protein, viral | IKKα activity |
| MC159 (Mollusum contagiosum virus) | Protein, viral | Reduced IKKα expression |
| NS5B (Hepatitis C virus) | Protein, viral | IKKβ activity |
| vIRF3 (KSHV) | Protein, viral | IKKβ activity |
| Cytomegalovirus | Protein, viral | Phosphorylation |
| HB-EGF (Heparin-binding epidermal | Protein | IKK activity |
| growth factor-like growth factor) | | |
| Hepatocyte growth factor | Protein | IKK activity |
| PAN1 (aka NALP2 or PYPAF2) | Protein | IKK activity |
| PTEN (tumor suppressor) | Protein | Activation of IKK |
| Interleukin-10 | Protein | Reduced IKKα and IKKβ expression |
| Anti-thrombin III | Protein | Phosphorylation |
| Chorionic gonadotropin | Protein | Phosphorylation |
| FHIT (Fragile histidine triad protein) | Protein | Phosphorylation |
| Interferon-α | Protein | Phosphorylation |
| SOCS1 | Protein | Phosphorylation |
| AGRO100 (G-quadruplex oligodeoxynucleotide) | Synthetic | NEMO binding |
| 2-amino-3-cyano-4-aryl-6-(2-hydroxy-phenyl) | Synthetic | IKKβ activity |
| pyridine derivatives | | |
| Acrolein | Synthetic | IKKβ activity |
| AS602868 | Synthetic | IKKβ activity |
| Aspirin, sodium salicylate | Synthetic | Phosphorylation, IKKβ |
| Dihydroxyphenylethanol | Synthetic | IKKβ activity |
| Epoxyquinone A monomer | Synthetic | IKKβ/DNA binding |
| Inhibitor 22 | Synthetic | IKKβ activity |
| MLB120 (small molecule) | Synthetic | IKKβ activity |
| Novel small-molecule inhibitor | Synthetic | IKKβ activity |
| BMS-345541 | Synthetic | IKK activity |
| CYL-19s and CYL-26z, two synthetic alpha- | Synthetic | IKK activity |
| methylene-gamma-butyrolactone derivatives | | |
| ACHP (2-amino-6-[2-(cyclopropyl-methoxy)-6- | Synthetic | IKKß activity (ATP analog) |
| hydroxyphenyl]-4-piperidin-4-yl nicotinonitrile | | |
| Compound A | Synthetic | IKKβ activity (ATP analog) |
| Compound 5 | Synthetic | IKK activity |
| Cyclopentenones | Synthetic | IKKβ activity |
| Jesterone dimer | Synthetic | IKKβ activity; DNA binding |
| PS-1145 (MLN1145) | Synthetic | IKKβ activity (ATP analog) |
| 2-[(aminocarbonyl)amino]-5-acetylenyl- | Synthetic | IKKβ activity |
| 3-thionphenecarboxamides | | |
| SC-514 | Synthetic | IKKβ activity |
| (Amino)imidazolylcarboxaldehyde | Synthetic | IKK activity |
| derivative | | |
| Amino-pyrimidine | Synthetic | IKK activity |
| Benzoimidazole derivative | Synthetic | IKK activity |
| CDDO-Me (synthetic triterpenoid) | Synthetic | IKK activity |
| CHS 828 (anticancer drug) | Synthetic | IKK activity |
| Diaylpyridine derivative | Synthetic | IKK activity |
| Imidazolylquinoline-carboxaldehyde derivative | Synthetic | IKK activity |
| Indolecarboxamide | Synthetic | IKK activity |
| LF15-0195 (analog of 15-deoxyspergualine) | Synthetic | IKK activity |
| ML120B | Synthetic | IKK activity |
| MX781 (retinoid antagonist) | Synthetic | IKK activity |
| NSAIDS | Synthetic | IKK activity |
| N-(4-hydroxyphenyl) retinamide | Synthetic | IKK activity |
| Pyrazolo[4,3-c]quinoline derivative | Synthetic | IKK activity |
| Pyridooxazinone derivative | Synthetic | IKK activity |
| Scytonemin | Synthetic | IKK activity |
| Survanta (Surfactant product) | Synthetic | IKK activity |
| Sulfasalazine | Synthetic | IKKα and IKKβ kinase activity |
| Sulfasalazine analogs | Synthetic | IKK kinase activity |
| Thalidomide | Synthetic | IKK activity |
| Azidothymidine (AZT) | Synthetic | Phosphorylation |
| BAY-11-7082 (E3((4-methylphenyl)-sulfonyl)-2- | Synthetic | Phosphorylation |
| propenenitrile) | | |
| BAY-11-7083 (E3((4-t-butylphenyl)-sulfonyl)-2- | Synthetic | Phosphorylation |
| propenenitrile) | | |
| Benzyl isothiocyanate | Synthetic | Phosphorylation |
| Carboplatin | Synthetic | Phosphorylation |
| Gabexate mesylate | Synthetic | Phosphorylation |
| Gleevec (Imatanib) | Synthetic | Phosphorylation |
| Hydroquinone | Synthetic | Phosphorylation |
| Ibuprofen | Synthetic | Phosphorylation |
| Inhaled isobutyl nitrite | Synthetic | Phosphorylation |
| Methotrexate | Synthetic | Phosphorylation |
| Monochloramine | Synthetic | Phosphorylation |
| Nafamostat mesylate | Synthetic | Phosphorylation |
| Statins (several) | Synthetic | Phosphorylation |
| THI 52 (1-naphthylethyl-6,7-dihydroxy-1,2,3,4- | Synthetic | Phosphorylation |
| tetrahydroisoquinoline) | | |
| tetrahydroisoquinoline) | Synthetic | Phosphorylation |
| 1,2,4-thiadiazolidine derivatives | Synthetic | Phosphorylation |
| YC-1 | Synthetic | Phosphory lation |
| Zinc | Inorganic complex | Degradation |
| Alachlor | Natural product | Degradation |
| Amentoflavone | Natural product | Degradation |
| Artemisia capillaris Thunb extract | Natural product | Degradation |
| Artemisia iwayomogi extract | Natural product | Degradation |
| L-ascorbic acid | Natural product | Degradation |
| Antrodia camphorata | Natural product | Degradation |
| Aucubin | Natural product | Degradation |
| Baicalein | Natural product | Degradation |
| β-lapachone | Natural product | Degradation |
| Blackberry extract | Natural product | Degradation |
| Buchang-tang | Natural product | Degradation |
| Capsaicin (8-methyl-N-vanillyl-6-nonenamide) | Natural product | Degradation |
| Catalposide | Natural product | Degradation |
| Cyclolinteinone (sponge sesterterpene) | Natural product | Degradation |
| Dihydroarteanniun | Natural product | Degradation |
| Docosahexaenoic acid | Natural product | Degradation |
| Emodin (3-methyl-1,6,8-trihydroxyanthraquinone) | Natural product | Degradation |
| Ephedrae herba (Mao) | Natural product | Degradation |
| Equol | Natural product | Degradation |
| Erbstatin (tyrosine kinase inhibitor) | Natural product | Degradation |
| Estrogen (E2) | Natural product | Degradation/and various other steps |
| Ethacrynic acid | Natural product | Degradation (and DNA binding) |
| Fosfomycin | Natural product | Degradation |
| Fungal gliotoxin | Natural product | Degradation |
| Gamisanghyulyunbueum | Natural product | Degradation |
| Genistein (tyrosine kinase inhibitor) | Natural product | Degradation; caspase cleavage of IκBα |
| Genipin | Natural product | Degradation |
| Glabridin | Natural product | Degradation |
| Glucosamine sulfate | Natural product | Degradation |
| Glutamine | Natural product | Degradation |
| Gumiganghwaltang | Natural product | Degradation |
| Isomallotochromanol and isomallotochromene | Natural product | Degradation |
| Kochia scoparia fruit (methanol extract) | Natural product | Degradation |
| Leflunomide metabolite (A77 1726) | Natural product | Degradation |
| Melatonin | Natural product | Degradation |
| 5'-methylthioadenosine | Natural product | Degradation |
| Midazolam | Natural product | Degradation |
| Momordin I | Natural product | Degradation |
| Mosla dianthera extract | Natural product | Degradation |
| Morinda officinalis extract | Natural product | Degradation |
| Opuntia ficus indica va saboten extract | Natural product | Degradation |
| β-Phenylethyl (PEITC) and 8-methylsulphinyloctyl | Natural product | Degradation |
| isothiocyanates (MSO) (watercress) | | |
| Platycodin saponins | Natural product | Degradation |
| Polymyxin B | Natural product | Degradation |
| Poncirus trifoliata fruit extract | Natural product | Degradation |
| Probiotics | Natural product | Degradation |
| Prostaglandin 15-deoxy-Δ(12,14)-PGJ(2) | Natural product | Degradation |
| Resiniferatoxin | Natural product | Degradation |
| Stinging nettle (Urtica dioica) plant extracts | Natural product | Degradation |
| Thiopental | Natural product | Degradation |
| Tipifarnib | Natural product | Degradation |
| Titanium | Natural product | Degradation |
| TNP-470 (angiogenesis inhibitor) | Natural product | Degradation |
| Trichomomas vaginalis infection | Natural product | Degradation |
| Triglyceride-rich lipoproteins | Natural product | Degradation |
| Ursodeoxycholic acid | Natural product | Degradation |
| Xanthium strumarium L. (methanol extract) | Natural product | Degradation |
| Penetratin | Peptide | Degradation |
| Vasoactive intestinal peptide | Peptide | Degradation (and CBP-RelA interaction) |
| K1L (Vaccinia virus protein) | Protein, viral | Degradation |
| Nef (HIV-1) | Protein, viral | Degradation |
| Vpu protein (HIV-1) | Protein, viral | TrCP ubiquitin ligase inhibitor |
| γ-glutamylcysteine synthetase | Protein | Degradation |
| Heat shock protein-70 | Protein | Degradation |
| ST2 (IL-1-like receptor secreted form) | Protein | Degradation |
| YopJ (encoded by Yersinia pseudotuberculosis) | Protein, bacterial | Deubiquintinase for IκBα |
| Activated protein C (APC) | Protein | Degradation |
| α-melanocyte-stimulating hormone (a-MSH) | Protein | Degradation |
| IL-13 | Protein | Degradation |
| Intravenous immunoglobulin | Protein | Degradation |
| Murr1 gene product | Protein | Degradation |
| Neurofibromatosis-2 (NF-2; merlin) protein | Protein | Degradation |
| Pituitary adenylate cyclase-activating polypeptide (PACAP) | Protein | Degradation |
| SAIF (Saccharomyces boulardii anti-inflammatory factor) | Protein | Degradation |
| Acetaminophen | Synthetic | Degradation |
| 1-Bromopropane | Synthetic | Degradation |
| Diamide (tyrosine phosphatase inhibitor) | Synthetic | Degradation |
| Dobutamine | Synthetic | Degradation |
| E-73 (cycloheximide analog) | Synthetic | Degradation |
| Ecabet sodium | Synthetic | Degradation |
| Gabexate mesylate | Synthetic | Degradation |
| Glimepiride | Synthetic | Degradation |
| Hypochlorite | Synthetic | Degradation |
| Losartin | Synthetic | Degradation |
| LY294002 (PI3-kinase inhibitor) | Synthetic | Degradation |
| [2-(4-morpholinyl)-8-phenylchromonel | | |
| Pervanadate (tyrosine phosphatase inhibitor) | Synthetic | Degradation |
| Phenylarsine oxide (PAO, tyrosine | Synthetic | Degradation |
| phosphatase inhibitor) | | |
| Phenytoin | Synthetic | Degradation |
| Sabaeksan | Synthetic | Degradation |
| U0126 (MEK inhibitor) | Synthetic | Degradation |
| Ro106-9920 (small molecule) | Synthetic | IκBα ubiqutination inhibitor |
| Low level laser therapy | — | Degradation |
| Electrical stimulation of vagus nerve | — | Degradation |
| Lactacystine, β-lactone | Natural product | Proteasome |
| Cyclosporin A | Natural product | Proteasome |
| ALLnL (N-acetyl-leucinyl-leucynil-norleucynal, MG101) | Peptide | Proteasome |
| LLM (N-acetyl-leucinyl-leucynil-methional) | Peptide | Proteasome |
| Z-LLnV (carbobenzoxyl-leucinyl-leucynil-norvalinal, MG115) | Peptide | Proteasome |
| Z-LLL (N-carbobenzoxyl-L-leucinyl-L-leucinyl-L-norleucinal, | Peptide | Proteasome |
| MG132) | | |
| Ubiquitin ligase inhibitors | Peptides | Proteasome |
| Boronic acid peptide | Synthetic | Proteasome |
| PS-341 (Bortezomib) | Synthetic | Proteasome |
| Salinosporamide A (1, NPI-0052) | Synthetic | Proteasome |
| FK506 (Tacrolimus) | Synthetic | Proteasome |
| Deoxyspergualin | Synthetic | Proteasome |
| Disulfiram | Synthetic | Proteasome |
| APNE (N-acetyl-DL-phenylalanine-b-naphthylester) | Synthetic | Protease |
| BTEE (N-benzoyl L-tyrosine-ethylester) | Synthetic | Protease |
| DCIC (3,4-dichloroisocoumarin) | Synthetic | Protease |
| DFP (diisopropyl fluorophosphate) | Synthetic | Protease |
| TPCK (N-α-tosyl-L-phenylalanine chloromethyl ketone) | Synthetic | Protease |
| TLCK (N-α-tosyl-L-lysine chloromethyl ketone) | Synthetic | Protease |
| Antrodia camphorata extract | Natural product | IκBα upregulation |
| Apigenin (4',5,7-trihydroxyflavone) | Natural product | IκBα upregulation |
| Glucocorticoids (dexamethasone, prednisone, methylprednisolone) | Natural product | IκBα upregulation |
| Human breast milk | Natural product | IκBα upregulation |
| α-pinene | Natural product | IκBα upregulation |
| Agastache rugosa leaf extract | Natural product | Nuclear translocation |
| Alginic acid | Natural product | Nuclear translocation |
| Astragaloside IV | Natural product | Nuclear translocation |
| Atorvastatin | Natural product | Nuclear translocation |
| 2',8″-biapigenin | Natural product | RelA nuclear translocation |
| Blue honeysuckle extract | Natural product | Nuclear translocation |
| Buthus martensi Karsch extract | Natural product | Nuclear translocation |
| Chiisanoside | Natural product | RelA Nuclear translocation |
| 15-deoxyspergualin | Natural product | Nuclear translocation |
| Eriocalyxin B | Natural product | Nuclear translocation |
| Gangliosides | Natural product | Nuclear translocation |
| Harpagophytum procumbens (Devil's Claw) extracts | Natural product | Nuclear translocation |
| Hirsutenone | Natural product | Nuclear translocation |
| JM34 (benzamide derivative) | Natural product | Nuclear translocation |
| KIOM-79 (combined plant extracts) | Natural product | Nuclear translocation |
| Leptomycin B (LMB) | Natural product | Nuclear translocation |
| Nucling | Natural product | RelA nuclear translocation |
| o,o'-bismyristoyl thiamine disulfide (BMT) | Natural product | Nuclear translocation |
| Oregonin | Natural product | RelA nuclear translocation |
| 1,2,3,4,6-penta-O-galloyl-β-D-glucose | Natural product | RelA nuclear translocation |
| Platycodi radix extract | Natural product | RelA nuclear translocation |
| Phallacidin | Natural product | Nuclear translocation |
| Piperine | Natural product | Nuclear translocation |
| Pitavastatin | Natural product | Nuclear translocation |
| Probiotics | Natural product | RelA nuclear translocation |
| Rhubarb aqueous extract | Natural product | RelA nuclear translocation |
| Selenomethionine | Natural product | Nuclear translocation |
| Salvia miltiorrhoza Bunge extract | Natural product | Nuclear translocation |
| ShenQi compound recipe | Natural product | RelA nuclear translocation |
| Sophorae radix extract | Natural product | Nuclear translocation |
| Sopoongsan | Natural product | Nuclear translocation |
| Sphondin (furanocoumarin derivative from Heracleum laciniatum) | Natural product | Nuclear translocation |
| Younggaechulgam-tang | Natural product | Nuclear translocation |
| Clarithromycin | Natural product | Nuclear NF-κB expression |
| 5F (from Pteri syeminpinnata L)) | Natural product | RelA expression |
| AT514 (serratamolide) | Natural product | RelA expression |
| oxidized 1-palmitoyl-2-arachidonoyl-sn-glycero-3-phosphorylcholine | Natural product | RelA expression |
| (OXPAPC) | | |
| Sorbus commixta cortex (methanol extract) | Natural product | RelA expression |
| Cantharidin | Natural product | NF-κB expression |
| Cornus officinalis extract | Natural product | NF-κB expression |
| Neomycin | Natural product | NF-κB expression |
| Paeoniflorin | Natural product | NF-κB expression |
| Rapamycin | Natural product | NF-κB expression |
| Sargassum hemiphyllum methanol extract | Natural product | NF-κB expression |
| Shenfu | Natural product | NF-κB expression |
| Tripterygium polyglycosides | Natural product | NF-κB expression |
| PN50 | Peptide | Nuclear translocation |
| Cell permeable NLS peptides | Peptide | Nuclear translocation |
| RelA peptides (P1 and P6) | Peptide | Nuclear translocation |
| Canine Distemper Virus | Protein, viral | Nuclear translocation |
| MNF (myxoma virus) | Protein, viral | Nuclear translocation |
| 3C protease (encephalomyocarditis virus) | Protein, viral | Rel A expression |
| ZUD protein | Protein | Activation of NF-κB; binds p105/RelA |
| HSCO (hepatoma protein) | Protein | Accelerates RelA nuclear export |
| β-amyloid protein | Protein | IκBα upregulation |
| Surfactant protein A (SP-A) | Protein | IκBα upregulation |
| DQ 65-79 (aa 65-79 of the alpha helix of the alpha-chain of the | Protein | IκBα upregulation and IKK inhibition |
| class II HLA molecule DQA03011) | | |
| C5a | Protein | IκBα upregulation |
| IL-10 | Protein | IκBα upregulation |
| IL-11 | Protein | IKKα; IκBα, IκBβ upregulation |
| IL-13 | Protein | IκBα upregulation |
| Fox1j | Protein | IκBβ upregulation |
| Glucorticoid-induced leucine zipper protein (GILZ) | Protein | Nuclear translocation |
| Heat shock protein 72 | Protein | Nuclear translocation |
| Retinoic acid receptor-related orphan | Protein | Nuclear translocation |
| receptor-alpha | | |
| TAT-SR-IκBα; MTS-SR-IκBα; p105-SR | Protein | Nuclear translocation |
| ZAS3 protein | Protein | RelA nuclear translocation; |
| | | DNA competition |
| RASSF1A gene overexpression | Protein | RelA nuclear expression |
| Onconase (Ranpirnase) | Protein | NF-κB expression |
| R-etodolac | Synthetic | IKBa upregulation |
| BMD (N(1)-Benzyl-4-methylbenzene-1,2-diamine) | Synthetic | Nuclear translocation |
| Carbaryl | Synthetic | Nuclear translocation |
| Indole-3-carbinol | Synthetic | Nuclear translocation |
| Dioxin | Synthetic | RelA nuclear translocation |
| Dehydroxymethylepoxyquinomicin | Synthetic | Nuclear translocation |
| (DHMEQ) | | |
| Dipyridamole | Synthetic | Nuclear translocation |
| Disulfiram | Synthetic | Nuclear translocation |
| Diltiazem | Synthetic | Nuclear translocation; induced |
| | | translocation of p50 dimers |
| Fluvastatin | Synthetic | Nuclear expression |
| JSH-23 (4-Methyl--(3-phenyl-propyl)-benzene-1,2-diamine | Synthetic | Nuclear translocation |
| KL-1156 (6-Hydroxy-7-methoxychroman-2-carboxylic acid | Synthetic | Nuclear translocation |
| phenylamide) | | |
| Leflunomide | Synthetic | RelA nuclear expression |
| Levamisole | Synthetic | Nuclear translocation |
| MEB (2-(4-morpholynl) ethyl butyrate hydrochloride) | Synthetic | Nuclear translocation |
| Rolipram | Synthetic | Nuclear translocation |
| SC236 (a selective COX-2 inhibitor) | Synthetic | Nuclear translocation |
| Triflusal | Synthetic | Nuclear expression |
| Volatile anesthetic treatment | Synthetic | Nuclear translocation |
| Moxifloxacin | Synthetic | RelA expression |
| Omapatrilat, enalapril, CGS 25462 | Synthetic | NF-κB expression |
| Estrogen enhanced transcript | mRNA | Nuclear translocation |
| Metals (chromium, cadmium, gold, lead, mercury, zinc, arsenic) | Inorganic Complex | DNA binding |
| Actinodaphine (from Cinnamomum insularimontanum) | Natural product | DNA binding |
| Anthocyanins (soybean) | Natural product | DNA binding |
| Arnica montana extract (sequiterpene lactones) | Natural product | DNA binding |
| Artemisinin | Natural product | DNA binding |
| Baicalein (5,6,7-trihydroxyflavone) | Natural product | DNA binding |
| Bambara groundnut (Vignea subterranean) | Natural product | DNA binding |
| β-lapachone (1,2-naphthoquinone) | Natural product | DNA binding |
| Biliverdin | Natural product | DNA binding |
| Brazilian | Natural product | DNA binding |
| Calcitriol (1a,25-dihydroxyvitamin D3) | Natural product | DNA binding |
| Campthothecin | Natural product | DNA binding |
| Cancer bush (Sutherlandia frutescens) | Natural product | DNA binding |
| Capsiate | Natural product | DNA binding |
| Catalposide (stem bark) | Natural product | DNA binding |
| Cat's claw bark (Uncaria tomentosa; Rubiaceae); Maca | Natural product | DNA binding |
| Cheongyeolsaseuptang | Natural product | DNA binding |
| Chitosan | Natural product | DNA binding |
| Chicory root (guaianolide 8-deoxylactucin) | Natural product | DNA binding |
| Chondrotin sulfate proteoglycan degradation product | Natural product | DNA binding |
| Clarithromycin | Natural product | DNA binding |
| Cloricromene | Natural product | DNA binding |
| Compound K (from Panax ginseng) | Natural product | DNA binding |
| Cortex cinnamomi extract | Natural product | DNA Binding |
| Cryptotanshinone | Natural product | DNA binding |
| Cytochalasin D | Natural product | DNA binding |
| DA-9201 (from black rice) | Natural product | DNA binding |
| Danshenshu | Natural product | DNA binding |
| Diterpenoids from Isodon rubescens or Liverwort Jungermannia | Natural product | DNA binding |
| ent-kaurane diterpenoids (Croton tonkinensis leaves) | Natural product | DNA binding |
| Epinastine hydrochloride | Natural product | DNA binding |
| Epoxyquinol A (fungal metabolite) | Natural product | DNA binding |
| Erythromycin | Natural product | DNA binding/transactivation |
| Evodiamine | Natural product | DNA binding |
| Fish oil feeding | Natural product | DNA binding |
| Fomes fomentarius methanol extracts | Natural product | DNA binding |
| Fucoidan | Natural product | DNA binding |
| Gallic acid | Natural product | DNA binding |
| Ganoderma lucidum (fungal dried spores or fruting body) | Natural product | DNA binding |
| Garcinol (fruit rind of Garcinia spp) | Natural product | DNA binding |
| Geranylgeraniol | Natural product | DNA binding |
| Ginkgolide B | Natural product | DNA binding |
| Glycyrrhizin | Natural product | DNA binding |
| Halofuginone | Natural product | DNA binding |
| Hematein (plant compound) | Natural product | DNA binding |
| Herbal compound 861 | Natural product | DNA binding |
| Hydroxyethyl starch | Natural product | DNA binding |
| Hydroxyethylpuerarin | Natural product | DNA binding |
| Hypericin | Natural product | DNA binding |
| Kamebakaurin | Natural product | DNA binding |
| Linoleic acid | Natural product | DNA binding |
| Lithospermi radix | Natural product | DNA binding |
| Macrolide antibiotics | Natural product | DNA binding |
| Mediterranean plant extracts | Natural product | DNA binding |
| 2-methoxyestradiol | Natural product | DNA binding; transactivation |
| 6-(Methylsulfinyl)hexyl isothiocyanate (Wasabi) | Natural product | DNA binding; transactivation |
| Nicotine | Natural product | DNA binding |
| Ochna macrocalyx bark extracts | Natural product | DNA binding |
| (diterpenoid from ) | Natural product | DNA binding |
| PC-SPES (8 herb mixture) | Natural product | DNA binding |
| 1,2,3,4,6-penta-O-galloyl-β-D-glucose | Natural product | DNA binding |
| Pepluanone | Natural product | DNA binding |
| Phyllanthus amarus extracts | Natural product | DNA binding |
| Plant compound A (a phenyl aziridine precursor) | Natural product | DNA binding and transactivation |
| Polyozellin | Natural product | DNA binding |
| Prenylbisabolane 3 | Natural product | DNA binding |
| Prostaglandin E2 | Natural product | DNA binding and RelA nuclear |
| | | translocation |
| Protein-bound polysaccharide (PSK) | Natural product | DNA binding |
| Quinic acid | Natural product | DNA binding |
| Sanggenon C | Natural product | DNA binding |
| Sesamin (from sesame oil) | Natural product | DNA binding |
| Shen-Fu | Natural Product | DNA binding |
| Silibinin | Natural product | DNA binding |
| Sinomenine | Natural product | DNA binding |
| Sword brake fern extract | Natural product | DNA binding |
| Tanacetum larvatum extract | Natural product | DNA binding |
| Tansinones (Salvia miltiorrhiza Bunge, Labiatae roots) | Natural product | DNA binding |
| Taurine + niacine | Natural product | DNA binding |
| Thiazolidinedione MCC-555 | Natural product | DNA binding |
| Trichostatin A | Natural product | RelA DNA binding |
| Triptolide (PG490, extract of Chinese herb) | Natural product | DNA binding |
| Tyrphostin AG-126 | Natural product | DNA binding |
| Ursolic acid | Natural product | DNA binding |
| Withaferin A | Natural product | DNA binding |
| Xanthohumol (a hops prenylflavonoid) | Natural product | DNA binding |
| Xylitol | Natural product | DNA binding |
| Yan-gan-wan | Natural product | DNA binding |
| Yin-Chen-Hao | Natural product | DNA binding |
| Yucca schidigera extract | Natural product | DNA binding |
| Ghrelin | Peptide | DNA binding |
| Peptide YY | Peptide | DNA binding |
| Rapamycin | Peptide | DNA binding |
| A238L IKB-like protein (African Swine Fever virus) | Protein, viral | DNA binding |
| C + V proteins (Sendai virus) | Protein, viral | DNA binding |
| E1B (Adenovirus) | Protein, viral | DNA binding |
| ICP27 (Herpes simplex virus-1) | Protein, viral | DNA binding |
| H4/N5 (Microplitis demolitor bracovirus) | Protein, viral | DNA binding |
| NS3/4A (Hepatitis C) | Protein, viral | DNA binding |
| Adiponectin | Protein | DNA binding |
| AIM2 (Absent in melanoma protein) overexpression | Protein | DNA binding |
| Angiopoietin-1 | Protein | DNA binding |
| Antithrombin | Protein | RelA-p300 interaction |
| AvrA protein (Salmonella) | Protein | DNA binding |
| β-catenin | Protein | DNA binding |
| Bromelain | Protein | DNA binding |
| Calcium/calmodulin-dependent kinase | Protein | DNA binding |
| kinase (CaMKK) (and increased intracellular calcium by | | |
| ionomycin, UTP and thapsigargin) | | |
| CD43 overexpression | Protein | DNA binding (RelA) |
| FLN29 overexpression | Protein | DNA binding |
| FLICE-Like Inhibitory Protein (FLIP) | Protein | DNA binding |
| G-120 (Ulmus davidiana Nakai glycoprotein) | Protein | DNA binding; IKB increases |
| Gax (homeobox protein) | Protein | DNA binding |
| HIV-1 Resistance Factor | Protein | DNA binding |
| Insulin-like growth factor binding protein-3 | Protein | DNA binding |
| Interleukin 4 (IL-4) | Protein | DNA binding |
| Leucine-rich effector proteins of Salmonella & Shigella | Protein | DNA binding |
| (SspH1 and IpaH9.8) | | |
| NDPP1 (CARD protein) | Protein | DNA binding |
| Overexpressed ZIP1 | Protein | DNA binding |
| p8 | Protein | DNA binding |
| p202a (nterferon inducible protein) | Protein | DNA binding by p65 and p50/p65; |
| | | increases p50 |
| p21 (recombinant) | Protein | DNA binding |
| PIAS1 (protein inhibitor of activatated STAT1) | Protein | RelA DNA binding |
| Pro-opiomelanocortin | Protein | DNA binding |
| PYPAF1 protein | Protein | DNA binding |
| Raf Kinase Inhibitor Protein (RKIP) | Protein | DNA binding |
| Rhus verniciflua Stokes fruits 36 kDa glycoprotein | Protein | DNA binding |
| Secretory leucoprotease inhibitor (SLPI) | Protein | DNA binding |
| Siah2 | Protein | DNA binding |
| SIRT1 Deacetylase overexpression | Protein | DNA binding |
| Siva-1 | Protein | DNA binding |
| Solana nigrum L. | Protein | DNA binding |
| Surfactant protein A | Protein | DNA binding |
| Tom1 (target of Myb-1) overexpression | Protein | DNA binding |
| Transdominant p50 | Protein | DNA binding |
| Uteroglobin | Protein | DNA binding |
| Vascular endothelial growth factor (VEGF) | Protein | DNA binding |
| ADP ribosylation inhibitors | Synthetic | DNA binding |
| (nicotinamide, 3-aminobenzamide) | | |
| 7-amino-4-methylcoumarin | Synthetic | DNA binding |
| Amrinone | Synthetic | DNA binding |
| Atrovastat (HMG-COA reductase inhibitor) | Synthetic | DNA binding |
| Benfotiamine (thiamine derivative) | Synthetic | DNA binding |
| Bisphenol A | Synthetic | DNA binding |
| Caprofen | Synthetic | DNA binding |
| Carbocisteine | Synthetic | DNA binding |
| Celecoxib and germcitabine | Synthetic | DNA binding |
| Cinnamaldehyde, 2-methoxycinnamaldehyde, | Synthetic | DNA binding |
| 2-hydroxycinnamaldehyde | | |
| Commerical peritoneal dialysis solution | Synthetic | DNA binding |
| CP Compound (6-Hydroxy-7-methoxychroman-2- | Synthetic | DNA binding |
| carboxylic acid phenylamide) | | |
| Cyanoguanidine CHS 828 | Synthetic | DNA binding |
| (κB site) Decoy oligonucleotides | Synthetic | DNA binding |
| Diarylheptanoid 7-(4'-hydroxy-3'-methoxyphenyl)- | Synthetic | DNA binding |
| 1-phenylhept-4-en-3-one | | |
| α-difluoromethylornithine (polyamine depletion) | Synthetic | DNA binding |
| DTD (4,10-dichloropyrido[5,6:4,5]thieno | Synthetic | DNA binding |
| [3,2-d':3,2-d]-1,2,3-ditriazine) | | |
| Evans Blue | Synthetic | DNA binding |
| Evodiamine | Synthetic | DNA binding |
| Fenoldopam | Synthetic | DNA binding |
| Fexofenadine hydrochloride | Synthetic | DNA binding |
| Fibrates | Synthetic | DNA binding |
| FK778 | Synthetic | DNA binding |
| Flunixin meglumine | Synthetic | DNA binding |
| Flurbiprofen | Synthetic | DNA binding |
| Hydroquinone (HQ) | Synthetic | DNA binding |
| IMD-0354 | Synthetic | DNA binding |
| JSH-21 (N1-Benzyl-4-methylbenzene-1,2-diamine) | Synthetic | DNA binding |
| KT-90 (morphine synthetic derivative) | Synthetic | DNA binding |
| Lovastatin | Synthetic | DNA binding |
| Mercaptopyrazine | Synthetic | DNA binding |
| Mevinolin, 5'-methylthioadenosine (MTA) | Synthetic | DNA binding |
| Monomethylfumarate | Synthetic | DNA binding |
| Moxifloxacin | Synthetic | DNA binding |
| Nicorandil | Synthetic | DNA binding |
| Nilvadipine | Synthetic | DNA binding |
| Nitric oxide-donating aspirin | Synthetic | DNA binding |
| Panepoxydone | Synthetic | DNA binding |
| Peptide nucleic acid-DNA decoys | Synthetic | DNA binding |
| Perindopril | Synthetic | DNA binding |
| 6(5H)-phenanthridinone and benzamide | Synthetic | DNA binding |
| Phenyl-N-tert-butylnitrone (PBN) | Synthetic | DNA binding |
| Pioglitazone (PPARgamma ligand) | Synthetic | DNA binding |
| Pirfenidone | Synthetic | DNA binding |
| Pyridine N-oxide derivatives | Synthetic | DNA binding |
| Quinadril (ACE inhibitor) | Synthetic | DNA binding |
| Raloxifene | Synthetic | RelA DNA binding |
| Raxofelast | Synthetic | DNA binding |
| Ribavirin | Synthetic | DNA binding |
| Rifamides | Synthetic | DNA binding |
| Ritonavir | Synthetic | DNA binding |
| Rosiglitazone | Synthetic | DNA binding |
| Roxithromycin | Synthetic | DNA binding |
| Santonin diacetoxy acetal derivative | Synthetic | DNA binding |
| Serotonin derivative (N-(p-coumaroyl) serotonin, SC) | Synthetic | DNA binding |
| Simvastatin | Synthetic | DNA binding |
| SM-7368 (small molecule) | Synthetic | DNA binding |
| T-614 (a methanesulfoanilide anti-arthritis inhibitor) | Synthetic | DNA binding |
| Sulfasalazine | Synthetic | DNA binding |
| SUN C8079 | Synthetic | DNA binding |
| Triclosan plus cetylpyridinium chloride | Synthetic | DNA binding |
| Tobacoo smoke | Synthetic | DNA binding |
| Verapamil | Synthetic | DNA binding |
| 8-acetoxy-5-hydroxyumbelliprenin | Natural product | Transactivation |
| Adenosine and cyclic AMP | Natural product | Transactivation |
| Artemisia sylvatica sesquiterpene lactones | Natural product | Transactivation (reporter assays) |
| α-zearalenol | Natural product | Transactivation |
| BSASM (plant extract mixture) | Natural product | Transactivation |
| Bifodobacteria | Natural product | Transactivation |
| | (Bacteria) | |
| Bupleurum fruticosum phenylpropanoids | Natural product | Transactivation |
| Blueberry and berry mix (Optiberry) | Natural product | Transactivation |
| 4'-demethyl-6-methoxypodophyllotoxin | Natural product | Transactivation |
| (lignan of Linum tauricum Willd. ssp. tauricum) | | |
| Cycloprodigiosin hycrochloride | Natural product | Transactivation |
| Eckol/Dieckol (seaweed E stolonifera) | Natural product | Transactivation |
| Extract of the stem bark of Mangifera indica L. | Natural product | NF-KB mRNA expression |
| Fructus Benincasae Recens extract | Natural product | Transactivation |
| Glucocorticoids (dexametasona, prednisone, | Natural product | Transactivation and increases |
| methylprednisolone) | | IκBα levels |
| Gypenoside XLIX (from Gynostemma pentaphyllum) | Natural product | Transactivation (PPAR-α-dependent) |
| Kwei Ling Ko (Tortoise shell-Rhizome jelly) | Natural product | Transactivation |
| Ligusticum chuanxiong Hort root | Natural product | Transactivation |
| Luteolin | Natural product | P65 transactivation |
| Manassantins A and B | Natural product | RelA transactivation |
| Mesuol | Natural product | RelA phosphorylation & transactivation |
| Nobiletin | Natural product | Transactivation |
| 4-phenylcoumarins (from Marila pluricostata) | Natural product | Transactivation |
| Phomol | Natural product | Transactivation |
| Psychosine | Natural product | Transactivation |
| Qingkailing and Shuanghuanglian (Chinese medicinal preparations) | Natural product | Transactivation |
| Saucerneol D and saucerneol E | Natural product | Transactivation |
| Smilax bockii warb extract (flavenoids) | Natural product | Transactivation |
| Trilinolein | Natural product | Transactivation |
| Uncaria tomentosum plant extract | Natural product | Transactivation |
| Witheringia solanacea leaf extracts | Natural product | Transactivation |
| Wortmannin (fungal metabolite) | Natural product | Transactivation |
| BZLF1 (EBV protein) | Protein, viral | Transactivation |
| SH gene products (Paromyxovirus) | Protein, viral | Transactivation |
| NRF (NF-κB repression factor) | Protein | Transactivation |
| PIAS3 | Protein | Transactivation |
| PTX-B (pertussis toxin binding protein) | Protein | RelA phosphorylation and transactivation |
| Antithrombin | Protein | RelA-p300 interaction |
| 17-allylamino-17-demethoxy geldanamycin | Synthetic | Transactivation |
| 6-aminoquinazoline derivatives | Synthetic | Transactivation |
| Chromene derivatives | Synthetic | Transactivation |
| D609 (phosphatidylcholine-phospholipase C inhibitor) | Synthetic | Transactivation |
| Dimethylfumarate (DMF) | Synthetic | Nuclear translocation |
| Ethyl 2-[(3-methyl-2,5-dioxo(3-pyrrolinyl)) pyrimidine-5-carboxylate | Synthetic | Transactivation |
| pyrimidine-5-carboxylate | | |
| Histidine | Synthetic | Transactivation |
| HIV-1 protease inhibitors (nelfinavir, ritonavir, or saquinavir) | Synthetic | Transactivation |
| Phenethylisothiocyanate | Synthetic | Transactivation |
| Pranlukast | Synthetic | Transactivation |
| RO31-8220 (PKC inhibitor) | Synthetic | Transactivation |
| SB203580 (p38 MAPK inhibitor) | Synthetic | Transactivation |
| Tetrathiomolybdate | Synthetic | Transactivation |
| Tranilast [N-(3,4-dimethoxycinnamoyl)anthranilic acid] | Synthetic | Transactivation |
| 3,4,5-trimethoxy-4'-fluorochalcone | Synthetic | Transactivation |
| Troglitazone | Synthetic | Transactivation |
| 9-aminoacridine (9AA) derivatives | Synthetic | RelA phosphorylation and transactivation |
| (including the antimalaria drug quinacrine) | | |
| Mesalamine | Synthetic | RelA phosphorylation & transactivation |
| Aged garlic extract (allicin) | | |
| 2-Amino-1-methyl-6-phenylimidazo | | |
| [4,5-b]pyridine (PhIP) | | |
| Anetholdithiolthione | | |
| Apocynin | | |
| Apple juice/extracts | | |
| Aretemisa p7F (5,6,3',5'-tetramethoxy | | |
| 7,4'-hydroxyflavone) | | |
| Astaxanthin | | |
| Benidipine | | |
| bis-eugenol | | |
| Bruguiera gymnorrhiza compounds | | |
| Butylated hydroxyanisole (BHA) | | |
| Caffeic Acid Phenethyl Ester (3,4- | | |
| dihydroxycinnamic acid, CAPE) | | |
| Carnosol | | |
| B-Carotene | | |
| Carvedilol | | |
| Catechol derivatives | | |
| Celasterol | | |
| Cepharanthine | | |
| Chlorophyllin | | |
| Chlorogenic acid | | |
| Cocoa polyphenols | | |
| Curcumin (Diferulolylmethane) | | |
| Dehydroevodiamine | | |
| Dehydroepiandrosterone (DHEA) and | | |
| DHEA-sulfate (DHEAS) | | |
| Dibenzylbutyrolactone lignans | | |
| Diethyldithiocarbamate (DDC) | | |
| Diferoxamine | | |
| Dihydroisoeugenol | | |
| Dihydrolipoic acid | | |
| Dilazep + fenofibric acid | | |
| Dimethyldithiocarbamates (DMDTC) | | |
| Dimethylsulfoxide (DMSO) | | |
| Disulfiram | | |
| Ebselen | | |
| Edaravone | | |
| EPC-K1 (phosphodiester compound of | | |
| vitamin E and vitamin C) | | |
| Epigallocatechin-3-gallate (EGCG; | | |
| green tea polyphenols) | | |
| Ergothioneine | | |
| Ethyl pyruvate (glutathione depletion) | | |
| Ethylene glycol tetraacetic acid (EGTA) | | |
| Extract of the stem bark of | | |
| Mangifera indica L. | | |
| Flavonoids (Crataegus; | | |
| Boerhaavia diffusa root; | | |
| xanthohumol) | | |
| Gamma-glutamylcysteine synthetase | | |
| (gamma-GCS) | | |
| Ganoderma lucidum polysaccharides | | |
| Garcinol (from extract of | | |
| Garcinia indica fruit rind) | | |
| Ginkgo biloba extract | | |
| Glutathione | | |
| Hematein | | |
| Hydroquinone | | |
| 23-hydroxyursolic acid | | |
| IRFI 042 (Vitamin E-like compound) | | |
| Iron tetrakis | | |
| Isovitexin | | |
| Kangen-karyu extract | | |
| Ketamine | | |
| L-cysteine | | |
| Lacidipine | | |
| Lazaroids | | |
| Ligonberries | | |
| α-lipoic acid | | |
| Lupeol | | |
| Magnolol | | |
| Maltol | | |
| Manganese superoxide dismutase | | |
| (Mn-SOD) | | |
| Mangiferin | | |
| Melatonin | | |
| Mulberry anthocyanins | | |
| Myricetin | | |
| Naringin | | |
| N-acetyl-L-cysteine (NAC) | | |
| Nacyselyn (NAL) | | |
| N-ethyl-maleimide (NEM) | | |
| Nitrosoglutathione | | |
| Nordihydroguaiaritic acid (NDGA) | | |
| Ochnaflavone | | |
| Orthophenanthroline | | |
| PMC (2,2,5,7,8-pentamethyl-6- | | |
| hydroxychromane) | | |
| Pentoxyifylline (1-(5'-oxohexyl) | | |
| 3,7-dimehylxanthine, PTX) | | |
| Phenolic antioxidants (Hydroquinone | | |
| and tert-butyl hydroquinone) | | |
| Phenylarsine oxide (PAO, tyrosine | | |
| phosphatase inhibitor) | | |
| Phyllanthus urinaria | | |
| Pyrithione | | |
| Pyrrolinedithiocarbamate (PDTC) | | |
| Quercetin (low concentrations) | | |
| Quinozolines | | |
| Rebamipide | | |
| Red wine | | |
| Ref-1 (redox factor 1) | | |
| Resveratrol | | |
| Rg(3), a ginseng derivative | | |
| Rotenone | | |
| Roxithromycin | | |
| S-allyl-cysteine (SAC, garlic compound) | | |
| Sauchinone | | |
| Spironolactone | | |
| Strawberry extracts | | |
| Taxifolin | | |
| Tempol | | |
| Tepoxaline (5-(4-chlorophenyl)-N- | | |
| hydroxy-(4-methoxyphenyl) -N-methyl- | | |
| 1H-pyrazole-3-propanamide) | | |
| Tetracylic A | | |
| α-tocopherol | | |
| α-torphryl acetate | | |
| α-torphryl succinate | | |
| Vitamin C | | |
| Vitamin B6 | | |
| Vitamin D | | |
| Vitamin E derivatives | | |
| Wogonin (5,7-dihydroxy-8- | | |
| methoxyflavone) | | |
| Yakuchinone A and B | | |
| Carbocysteine | Mucus Regulation | |
| CGS 25462 | Neutral endopeptidase | |
| CHS 828 | DNA synthesis | |
| Clarithromycin | Bacterial 50S ribosome | |
| Dipyridamole | Adenosine deaminase/PDE | |
| Disulfiram | Acetaldehyde | |
| | dehydrogenase | |
| Fenoldopam | Dopamine receptor | |
| Fibrates | PPARa | |
| Fluvastin | HMG-CoA | |
| | reductase | |
| Gleevec | BCR-ABL | |
| Leflunomide | Dehydroorotate | |
| | dehydrogenase | |
| Moxifloxacin | Bacterial DNA Gyrase | |
| Perindopril | Angiotensin converting | |
| | enzyme | |
| Raloxifene | Estrogen Receptor | |
| Rapamycin | FK-binding protein 12 | |
| Ritonavir | HIV-1 protease | |
| Tetrathiomolybdate | Anti-copper | |
| Triflusal | Cyclooxygenase-1 | |
| Troglitazone | PPAR-Y | |

One or more of the NF-κB inhibitors in Table 1 may be specifically excluded in the methods and compositions of the disclosure.

### IV. ANTIGENS

The term “antigen” as used herein refers to a molecule against which a subject can initiate a humoral and/or cellular immune response. Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. In certain compositions and methods of the disclosure, the antigen is a peptide.

The inventors have demonstrated that the hybrid molecules disclosed herein reduce systemic toxicity to baseline levels while maintaining the adjuvanticity in a vaccine formulation. Antigens useful in methods and compositions of the disclosure include, for example, antigenic components from Anthrax, Cancer, Chikungunya, Dengue (1,2,3,4-Dengue Fever), Diphtheria, E. coli, Shiga toxin-producing (STEC), Ebola, Non-Polio Enterovirus, Enterovirus D68 (EV-D68), Gonorrhea, Hepatitis A (Hep A), Hepatitis B (Hep B), Hepatitis C (Hep C), Hepatitis D (Hep D), Hepatitis E (Hep E), Herpes, Shingles, HIV, HPV, Influenza, Malaria, Measles, Viral Meningitis, Bacterial Menigitis, Mumps, Norovirus, Pertussis, Plague; Bubonic, Septicemic, Pneumonic, Pneumococcal Disease, Poliomyelitis (Polio), Pustular Rash diseases (Small pox, monkeypox, cowpox), Q-Fever, Rabies, Salmonellosis gastroenteritis (Salmonella), Severe Acute Respiratory Syndrome, Shigellosis gastroenteritis (Shigella), Smallpox, Tetanus, Tuberculosis, Varicella (Chickenpox), Viral Hemorrhagic Fever (Ebola, Lassa, Marburg), West Nile Virus, Yellow Fever, Yersenia (Yersinia), and Zika Virus Infection. It is contemplated that one or more of the antigens and antigenic components listed in this paragraph are specifically excluded.

Further examples of antigens useful in the methods and compositions of the disclosure are provided below and throughout the disclosure.

### A. Viral Antigens

Examples of viral antigens include, but are not limited to, retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B. and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components. See Fundamental Virology, Second Edition, e's. Fields, B. N. and Knipe, D. M. (Raven Press, New York, 1991) for additional examples of viral antigens. It is contemplated that one or more of the antigens and antigenic components listed in this paragraph are specifically excluded.

### B. Bacterial Antigens

Bacterial antigens which can be used in the compositions and methods of the disclosure include, but are not limited to, pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; hemophilus influenza bacterial antigens such as capsular polysaccharides and other hemophilus influenza bacterial antigen components; anthrax bacterial antigens such as anthrax protective antigen and other anthrax bacterial antigen components; rickettsiae bacterial antigens such as romps and other rickettsiae bacterial antigen component. Also included with the bacterial antigens described herein are any other bacterial, mycobacterial, Mycoplasmal, rickettsial, or chlamydial antigens. It is contemplated that one or more of the antigens and antigenic components listed in this paragraph are specifically excluded in methods and compositions of the disclosure.

### C. Fungal Antigens

Fungal antigens which can be used in the compositions and methods of the disclosure include, but are not limited to, Candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. It is contemplated that one or more of the antigens and antigenic components listed in this paragraph are specifically excluded in methods and compositions of the disclosure.

### D. Parasite Antigens

Examples of protozoa and other parasitic antigens include, but are not limited to, Plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; Leishmania major and other Leishmaniae antigens such as gp63, lipophosphoglycan and its associated protein and other Leishmanial antigen components; and Trypanosoma cruzi antigens such as the 75-77 kDa antigen, the 56 kDa antigen and other Trypanosomal antigen components. It is contemplated that one or more of the antigens and antigenic components listed in this paragraph are specifically excluded in methods and compositions of the disclosure.

### E. Tumor Antigens

Tumor antigens which can be used in the compositions and methods of the disclosure include, but are not limited to, telomerase components; multidrug resistance proteins such as P-glycoprotein; MAGE-1, alpha fetoprotein, carcinoembryonic antigen, mutant p53, immunoglobulins of B-cell derived malignancies, fusion polypeptides expressed from genes that have been juxtaposed by chromosomal translocations, human chorionic gonadotrpin, calcitonin, tyrosinase, papillomavirus antigens, gangliosides or other carbohydrate-containing components of melanoma or other tumor cells. It is contemplated by the disclosure that antigens from any type of tumor cell can be used in the compositions and methods described herein. It is contemplated that one or more of the antigens and antigenic components listed in this paragraph are specifically excluded in methods and compositions of the disclosure.

### F. Antigens Relating to Autoimmunity

Antigens involved in autoimmune diseases, allergy, and graft rejection can be used in the compositions and methods of the disclosure. For example, an antigen involved in any one or more of the following autoimmune diseases or disorders can be used in the present disclosure: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. An antigen can also be an altered peptide ligand useful in treating an autoimmune disease. It is contemplated that one or more of the antigens and antigenic components listed in this paragraph are specifically excluded in methods and compositions of the disclosure. It is further contemplated that autoantigens are specifically excluded in methods and compositions of the disclosure.

Examples of miscellaneous antigens which can be used in the compositions and methods of the disclosure include endogenous hormones such as luteinizing hormone, follicular stimulating hormone, testosterone, growth hormone, prolactin, and other hormones, drugs of addiction such as cocaine and heroin, and idiotypic fragments of antigen receptors such as Fab-containing portions of an anti-leptin receptor antibody.

### V. PRR AND TLR AGONISTS

Certain aspects of the present disclosure are directed to pattern recognition receptor (PRR) agonists. A PRR agonist describes any molecule that, directly or indirectly, activates a PRR or stimulates PRR signaling. PRRs include cell surface receptors (e.g., toll-like receptor (TLR) agonists) and intracellular receptors (e.g., RIG-I-like receptors). Examples of PRRs which may be targeted by agonists of the present disclosure include NOD-like receptors, RIG-I-like receptors, STING receptors, and toll-like receptors. In some embodiments, disclosed herein are PRR agonists, wherein a PRR agonist is a NOD-like receptor agonist, a RIG-I-like receptor agonist, a STING agonist, or a TLR agonist. In some embodiments, a PRR agonist of the present disclosure is a TLR agonist.

Aspects of the present disclosure relate to TLR agonists, including polymers comprising a TLR agonist or derivative thereof. A TLR agonist may be any molecule that, directly or indirectly, activates a TLR and/or stimulates TLR signaling. In some cases, a TLR agonist is a molecule that binds directly to a TLR. In some aspects, disclosed are immunomodulators comprising one or more TLR agonists linked by a polypeptide backbone.

In some embodiments, the TLR agonist is one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111:B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam₂CSK₄; lipoarabinomannan or lipomannan from M. smegmatis; triacylated lipoproteins such as Pam₃CSK₄; lipoproteins such as MALP-2 and MALP-404 from Mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™ Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CLO97, 2Bxy, R848, or R848 VacciGrade™), TLR9 (e.g., CpG ODNs); and TLR11 (e.g., Toxoplasma gondii Profilin). In some embodiments, the TLR agonist is an amphiphilic TLR agonist. In some embodiments, the TLR agonist is a TLR 2/6 agonist, for example Pam₂CSK₄ or Pam₃CSK₄. In some embodiments, the TLR agonist is a hydrophobic TLR agonist. In some embodiments, the TLR agonist is a TLR 7, TLR 8, or TLR 7/8 agonist, for example 2Bxy or imidazoquinoline. In some aspects, a TLR agonist of the disclosure is imidazoquinoline. In certain embodiments, the TLR agonist is a specific agonist listed above. Derivatives of any of the TLR agonists listed above are also contemplated herein, and TLR agonists of the disclosure encompass any derivative of the molecules listed above having TLR agonist activity.

In further embodiments, the TLR agonist is one that agonizes either one TLR or two TLRs specifically. In some embodiments, linked TLR agonists comprise different types of TLR agonists (e.g., TLR agonists capable of activating different classes of TLRs). Alternatively, linked TLR agonists may comprise the same type of TLR agonist.

In some embodiments, disclosed herein are small molecule compounds suitable for use as TLR agonists. Examples of small molecule TLR agonists include compounds having a 2-aminopyridine fused to a five membered nitrogen-containing heterocyclic ring. Such compounds include, for example, imidazoquinoline amines including but not limited to substituted imidazoquinoline amines such as, for example, aminoalkyl-substituted imidazoquinoline amines, amide-substituted imidazoquinoline amines, sulfonamide-substituted imidazoquinoline amines, urea-substituted imidazoquinoline amines, aryl ether-substituted imidazoquinoline amines, heterocyclic ether-substituted imidazoquinoline amines, amido ether-substituted imidazoquinoline amines, sulfonamido ether-substituted imidazoquinoline amines, urea-substituted imidazoquinoline ethers, and thioether-substituted imidazoquinoline amines; tetrahydroimidazoquinoline amines including but not limited to amide-substituted tetrahydroimidazoquinoline amines, sulfonamide-substituted tetrahydroimidazoquinoline amines, urea-substituted tetrahydroimidazoquinoline amines, aryl ether-substituted tetrahydroimidazoquinoline amines, heterocyclic ether-substituted tetrahydroimidazoquinoline amines, amido ether-substituted tetrahydroimidazoquinoline amines, sulfonamido ether-substituted tetrahydroimidazoquinoline amines, urea-substituted tetrahydroimidazoquinoline ethers, and thioether-substituted tetrahydroimidazoquinoline amines; imidazopyridine amines including but not limited to amide-substituted imidazopyridine amines, sulfonamido-substituted imidazopyridine amines, urea-substituted imidazopyridine amines; aryl ether-substituted imidazopyridine amines, heterocyclic ether-substituted imidazopyridine amines, amido ether-substituted imidazopyridine amines, sulfonamido ether-substituted imidazopyridine amines, urea-substituted imidazopyridine ethers, and thioether-substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines.

In certain embodiments, the TLR agonist is an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the TLR agonist is a sulfonamide-substituted imidazoquinoline amine. In alternative embodiments, the TLR agonist can be a urea-substituted imidazoquinoline ether. In another alternative embodiment, the TLR agonist can be an aminoalkyl-substituted imidazoquinoline amine. In one particular embodiment, the TLR agonist is 4-amino-α,α,2-trimethyl-1H-imidazo[4,5-c]quinolin-1-ethanol. In an alternative particular embodiment, the TLR agonist is N-(2-{2-[4-amino-2-(2-methoxyethyl)-1H-imidazo[4,5-c]quinolin-1-yl]ethoxy}ethyl)-N-methylmorpholine-4-carboxamide. In another alternative embodiment, the TLR agonist is 1-(2-amino-2-methylpropyl)-2-(ethoxymethyl)-1H-imidazo[4,5-c]quinolin-4-amine. In another alternative embodiment, the TLR agonist is N-[4-(4-an-no-2-ethyl-1H-imidazo[4,5-c]quinolin-1-yl)butyl]methanesulfonamide. In yet another alternative embodiment, the TLR agonist is N-[4-(4-amino-2-propyl-1H-imidazo[4,5-c]quinolin-1-yl)butyl]methanesulfonamide.

In certain embodiments, the TLR agonist may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an aminoalkyl-substituted imidazoquinoline amine, an amide-substituted imidazoquinoline amine, a sulfonamide-substituted imidazoquinoline amine, a urea-substituted imidazoquinoline amine, an aryl ether-substituted imidazoquinoline amine, a heterocyclic ether-substituted imidazoquinoline amine, an amido ether-substituted imidazoquinoline amine, a sulfonamido ether-substituted imidazoquinoline amine, a urea-substituted imidazoquinoline ether, or a thioether-substituted imidazoquinoline amines.

### VI. AMINO ACIDS

The term “amino acid” refers to natural amino acids, non-natural amino acids (also “unnatural amino acids”), and amino acid analogs, all in their D and L stereoisomers, unless otherwise indicated, if their structures allow such stereoisomeric forms. An amino acid, may be e.g., of the formula:

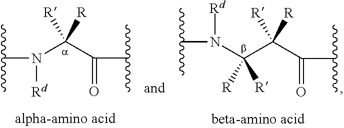

wherein each instance of R and R′ independently are selected from the group consisting of hydrogen, optionally substituted aliphatic, optionally substituted heteroaliphatic, optionally substituted aryl, optionally substituted heteroaryl, and R^{d} is hydrogen or an amino protecting group. Amino acids encompassed by the above two formulae include, without limitation, natural alpha-amino acids such as D- and L-isomers of the 20 common naturally occurring alpha-amino acids found in polypeptides and proteins (e.g., A, R, N, C, D, Q, E, G, H, I, L, K, M, F, P, S, T, W, Y, V, as depicted in Table 2 below), non-natural alpha-amino acids (examples of which are depicted in Table 3 below), natural beta-amino acids (e.g., beta-alanine), and unnatural beta-amino acids.

TABLE 2
Natural Amino Acids

| Amino Acid | R | R’ |
|---|---|---|
| L-Alanine (A) | —CH₃ | —H |
| L-Arginine (R) | —CH₂CH₂CH₂—NHC(═NH)NH₂ | —H |
| L-Asparagine (N) | —CH₂C(═O)NH₂ | —H |
| L-Aspartic acid (D) | —CH₂CO₂H | —H |
| L-Cysteine (C) | —CH₂SH | —H |
| L-Glutamic acid (E) | —CH₂CH₂CO₂H | —H |
| L-Glutamine (Q) | —CH₂CH₂C(=O)NH₂ | —H |
| Glycine (G) | —H | —H |
| L-Histidine (H) | —CH₂-2-(1H-imidazole) | —H |
| L-Isoleucine (I) | -sec-butyl | —H |
| L-Leucine (L) | -iso-butyl | —H |
| L-Lysine (K) | —CH₂CH₂CH₂CH₂NH₂ | —H |
| L-Methionine (M) | —CH₂CH₂SCH₃ | —H |
| L-Phenylalanine (F) | —CH₂Ph | —H |
| L-Proline (P) | -2-(pyrrolidine) | —H |
| L-Serine (S) | —CH₂OH | —H |
| L-Threonine (T) | —CH₂CH(OH)(CH₃) | —H |
| L-Tryptophan (W) | —CH₂-3-(1H-indole) | —H |
| L-Tyrosine (Y) | —CH₂-(p-hydroxyphenyl) | —H |
| L-Valine (V) | -isopropyl | —H |

TABLE 3
Non-natural Amino Acids

| Amino Acid | R | R’ |
|---|---|---|
| D-Alanine | —H | —CH₃ |
| D-Arginine | —H | —CH₂CH₂CH₂—NHC(═NH)NH₂ |
| D-Asparagine | —H | CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | CH═CH2 | —CH═CH2 |

| Amino Acid | R and R’ are equal to: |
|---|---|
| α-methyl-Alanine (Aib) | —CH₃, —CH₃ |
| α-methyl-Arginine | —CH₃, —CH₂CH₂CH₂—NHC(═NH)NH2 |
| α-methyl-Asparagine | —CH₃, —CH₂C(═O)NH₂ |
| α-methyl-Aspartic acid | —CH₃, —CH₂CO₂H |
| α-methyl-Cysteine | —CH₃, —CH₂SH |
| α-methyl-Glutamic acid | —CH₃, —CH₂CH₂CO₂H |
| α-methyl-Glutamine | —CH₃, —CH₂CH₂C(═O)NH₂ |
| α-methyl-Histidine | —CH₃, —CH₂-2-(1H-imidazole) |
| α-methyl-Isoleucine | —CH₃, -sec-butyl |
| α-methyl-Leucine | —CH₃, -iso-butyl |
| α-methyl-Lysine | —CH₃, —CH₂CH₂CH₂CH₂NH₂ |
| α-methyl-Methionine | —CH₃, —CH₂CH₂SCH₃ |
| α-methyl-Phenylalanine | —CH₃, —CH₂Ph |
| α-methyl-Proline | —CH₃, -2-(pyrrolidine) |
| α-methyl-Serine | —CH₃, —CH₂OH |
| α-methyl-Threonine | —CH₃, —CH₂CH(OH)(CH₃) |
| α-methyl-Tryptophan | —CH₃, —CH₂-3-(1H-indole) |
| α-methyl-Tyrosine | —CH₃, —CH₂-(p-hydroxyphenyl) |
| α-methyl-Valine | —CH₃, -isopropyl |
| Di-vinyl Norleucine | —CH═CH₂, —CH═CH₂, —H, —CH₂CH₂CH₂CH₃ |

There are many known unnatural amino acids any of which may be included in the polypeptides, polypeptide backbones, polymers, and compounds of the present invention. See, for example, S. Hunt, The Non-Protein Amino Acids: In Chemistry and Biochemistry of the Amino Acids, edited by Gl. C. Barrett, Chapman and Hall, 1985; incorporated by reference in its entirety. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine.

Certain unnatural amino acids may be included in a polypeptide chain for peptide stapling or stitching. These unnatural amino acids include a terminal unsaturated moiety, such as a double or triple bond. Exemplary amino acids with terminal olefinic unsaturation include, but are not limited to, —(CH₂)_{g}—S—(CH₂)_{g}CH═CH₂; —(CH₂)_{g}—O—(CH₂)_{g}CH═CH₂; —(CH₂)_{g}—NH—(CH₂)_{g}CH═CH₂; —(CH₂)_{g}—(C═O)—S—(CH₂)_{g}CH═CH₂; —(CH₂)_{g}—(C═O)—O—(CH₂)_{g}CH═CH₂; —(CH₂)_{g}—(C═O)—NH—(CH₂)_{g}CH═CH₂; —CH₂CH₂CH₂CH₂—NH—(CH₂)_{g}CH═CH₂; —(C₆H₅)-p-O—(CH₂)_{g}CH═CH₂; —CH(CH₃)—O—(CH₂)_{g}CH═CH₂; —CH₂CH(—O—CH═CH₂)(CH₃); -histidine-N((CH₂)_{g}CH═CH₂); -tryptophan-N((CH₂)_{g}CH═CH₂); and (CH₂)_{g+1}(CH═CH₂), wherein each instance of g is, independently, 0 to 10, inclusive. Specific amino acids with terminal unsaturation are further described and depicted herein.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

### VII. PHARMACEUTICAL COMPOSITIONS

The disclosure relates to TLR agonists, including dimers comprising a TLR agonist or derivative thereof. A TLR agonist may be any molecule that, directly or indirectly, activates a TLR and/or stimulates TLR signaling. In some cases, a TLR agonist is a molecule that binds directly to a TLR. In some cases, TLR agonists of the present disclosure are linked, for example, by a polyethylene glycol (PEG) or other molecular linker. TLR agonists may be formulated into nanoparticles, optionally with one or more co-assembly agents (e.g., functionalized polymers).

The TLR agonist may be one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111:B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam₂CSK₄; lipoarabinomannan or lipomannan from M. smegmatis; triacylated lipoproteins such as Pam₃CSK₄; lipoproteins such as MALP-2 and MALP-404 from Mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™ Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CLO97, 2Bxy, R848, or R848 VacciGrade™) TLR9 (e.g., CpG ODNs); and TLR11 (e.g., Toxoplasma gondii Profilin). The TLR agonist may be an amphiphilic TLR agonist. The TLR agonist may be a TLR 2/6 agonist, for example Pam₂CSK₄ or Pam₃CSK₄. The TLR agonist may be a hydrophobic TLR agonist. The TLR agonist may be a TLR 7, TLR 8, or TLR 7/8 agonist, for example 2Bxy. The TLR agonist may be a specific agonist listed above. The TLR agonist may be one that agonizes either one TLR or two TLRs specifically. Linked TLR agonists may comprise different types of TLR agonists (e.g., TLR agonists capable of activating different classes of TLRs). For example, a linked TLR agonist may comprise a TLR 2/6 agonist and a TLR 7 agonist covalently attached by a molecular linker. Alternatively, linked TLR agonists may comprise the same type of TLR agonist.

Disclosed herein are small molecule compounds suitable for use as TLR agonists. Examples of small molecule TLR agonists include compounds having a 2-aminopyridine fused to a five membered nitrogen-containing heterocyclic ring. Such compounds include, for example, imidazoquinoline amines including but not limited to substituted imidazoquinoline amines such as, for example, aminoalkyl-substituted imidazoquinoline amines, amide-substituted imidazoquinoline amines, sulfonamide-substituted imidazoquinoline amines, urea-substituted imidazoquinoline amines, aryl ether-substituted imidazoquinoline amines, heterocyclic ether-substituted imidazoquinoline amines, amido ether-substituted imidazoquinoline amines, sulfonamido ether-substituted imidazoquinoline amines, urea-substituted imidazoquinoline ethers, and thioether-substituted imidazoquinoline amines; tetrahydroimidazoquinoline amines including but not limited to amide-substituted tetrahydroimidazoquinoline amines, sulfonamide-substituted tetrahydroimidazoquinoline amines, urea-substituted tetrahydroimidazoquinoline amines, aryl ether-substituted tetrahydroimidazoquinoline amines, heterocyclic ether-substituted tetrahydroimidazoquinoline amines, amido ether-substituted tetrahydroimidazoquinoline amines, sulfonamido ether-substituted tetrahydroimidazoquinoline amines, urea-substituted tetrahydroimidazoquinoline ethers, and thioether-substituted tetrahydroimidazoquinoline amines; imidazopyridine amines including but not limited to amide-substituted imidazopyridine amines, sulfonamido-substituted imidazopyridine amines, urea-substituted imidazopyridine amines; aryl ether-substituted imidazopyridine amines, heterocyclic ether-substituted imidazopyridine amines, amido ether-substituted imidazopyridine amines, sulfonamido ether-substituted imidazopyridine amines, urea-substituted imidazopyridine ethers, and thioether-substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines.

The TLR agonist may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

The TLR agonist may be a sulfonamide-substituted imidazoquinoline amine. The TLR agonist can be a urea-substituted imidazoquinoline ether. The TLR agonist can be an aminoalkyl-substituted imidazoquinoline amine. The TLR agonist may be 4-amino-α,α,2-trimethyl-1H-imidazo[4,5-c]quinolin-1-ethanol. The TLR agonist may be N-(2-{2-[4-amino-2-(2-methoxyethyl)-1H-imidazo[4,5-c]quinolin-1-yl]ethoxy}ethyl)-N-methylmorpholine-4-carboxamide. The TLR agonist may be 1-(2-amino-2-methylpropyl)-2-(ethoxymethyl)-1H-imidazo[4,5-c]quinolin-4-amine. The TLR agonist may be N-[4-(4-an-no-2-ethyl-1H-imidazo[4,5-c]quinolin-1-yl)butyl]methanesulfonamide. The TLR agonist may be N-[4-(4-amino-2-propyl-1H-imidazo[4,5-c]quinolin-1-yl)butyl]methanesulfonamide.

The TLR agonist may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an aminoalkyl-substituted imidazoquinoline amine, an amide-substituted imidazoquinoline amine, a sulfonamide-substituted imidazoquinoline amine, a urea-substituted imidazoquinoline amine, an aryl ether-substituted imidazoquinoline amine, a heterocyclic ether-substituted imidazoquinoline amine, an amido ether-substituted imidazoquinoline amine, a sulfonamido ether-substituted imidazoquinoline amine, a urea-substituted imidazoquinoline ether, or a thioether-substituted imidazoquinoline amines.

### VIII. PHARMACEUTICAL COMPOSITIONS

Administration of the compositions will typically be via any common route. This includes, but is not limited to parenteral, orthotopic, intradermal, subcutaneous, intramuscular, intraperitoneal, intranasal, or intravenous injection. A vaccine composition may be inhaled (e.g., U.S. Pat. No. 6,651,655, which is specifically incorporated by reference). Additional formulations which are suitable for other modes of administration include oral formulations. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, for example about 25% to about 70%.

Typically, compositions are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of an antibody are applicable. These are believed to include oral application on a solid physiologically acceptable base or in a physiologically acceptable dispersion, parenterally, by injection and the like. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2 day to twelve week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The hybrid molecules can be formulated for parenteral administration, e.g., formulated for injection via the intravenous, intradermal, intramuscular, sub-cutaneous, or even intraperitoneal routes. The composition may be administered by intradermal injection. The composition may be administered by intravenous injection. The composition may be administered by intramuscular injection. Compositions of the disclosure can be prepared as injectables, either as liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active ingredients in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### IX. METHODS OF TREATMENT

As discussed above, the compositions and methods of using these compositions can treat a subject (e.g., prevent an infection, evoke a robust immune response to an antigen, or reduce or prevent tumor proliferation) having, suspected of having, or at risk of developing an infection, cancer, or related disease.

As used herein the phrase “immune response” or its equivalent “immunological response” refers to a humoral (antibody mediated), cellular (mediated by antigen-specific T cells or their secretion products) or both humoral and cellular response directed against a protein, peptide, or polypeptide of the invention in a recipient patient. Treatment or therapy can be an active immune response induced by administration of immunogen or a passive therapy effected by administration of antibody, antibody containing material, or primed T-cells.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject. As used herein and in the claims, the terms “antibody” or “immunoglobulin” are used interchangeably.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

The methods may include treatment for or prevention of a disease or condition caused by a pathogen. Furthermore, in some examples, treatment comprises administration of other agents commonly used against viral infection, such as one or more antiviral or antiretroviral compounds.

The therapeutic compositions are administered in a manner compatible with the dosage formulation, and in such amount as will be therapeutically effective. The quantity to be administered depends on the subject to be treated. Precise amounts of active ingredient required to be administered depend on the judgment of the practitioner. Suitable regimes for initial administration and boosters are also variable, but are typified by an initial administration followed by subsequent administrations.

The manner of application may be varied widely. Any of the conventional methods for administration of a polypeptide therapeutic are applicable. These are believed to include oral application on a solid physiologically acceptable base or in a physiologically acceptable dispersion, parenterally, by injection and the like. The dosage of the composition will depend on the route of administration and will vary according to the size and health of the subject.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

A subject may be administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 445, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 micrograms, mg, jg/kg, or mg/kg (or any range derivable therein), of hybrid molecule or composition.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. The patient may be administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of an the condition have disappeared or been reduced or after 6, 12, 18, or 24 hours or 1, 2, 3, 4, or 5 days after symptoms of an infection have disappeared or been reduced.

### X. COMBINATION THERAPY

The compositions and related methods, particularly administration of a composition comprising a hybrid molecule, may also be used in combination with the administration of one or more additional therapies.

A therapy may be used in conjunction with antiviral or anti-retroviral treatment. A therapy may be used in conjunction with an anti-cancer treatment (e.g., chemotherapeutic, cancer immunotherapeutic, etc.). The therapy may precede or follow the other agent treatment by intervals ranging from minutes to weeks. Where the other agents and/or proteins or polynucleotides are administered separately, one would generally ensure that a significant period of time did not expire between the time of each delivery, such that the therapeutic composition would still be able to exert an advantageously combined effect on the subject. In such instances, it is contemplated that one may administer both modalities within about 12-24 h of each other, for example within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

A vaccine may be administered as part of a prime/boost strategy. A priming vaccine dose can be administered in any of the methods described herein. A vaccine boost can be administered through the use of a second vaccine, either of the same type or from a different type of vaccine. Examples of such different vaccines include naked DNA vaccines or a recombinant poxvirus.

Various combinations of therapy may be employed, for example adjuvant is “A” and NFkB inhibitor is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions to a patient/subject will follow general protocols for the administration of such compounds, taking into account the toxicity, if any, of the composition. It is expected that the treatment cycles would be repeated as necessary. It is also contemplated that various standard therapies, such as hydration, may be applied in combination with the

### XI. CANCER THERAPY

The disclosed methods may comprise administering a cancer therapy to a subject or patient. The cancer therapy may comprise a local cancer therapy. The cancer therapy may exclude a systemic cancer therapy. The cancer therapy may exclude a local therapy. The cancer therapy may comprise a local cancer therapy without the administration of a system cancer therapy. The cancer therapy may comprise administering a dimer of the present disclosure. The cancer therapy may comprise a radiotherapy. The cancer therapy may comprise a chemotherapy. The cancer therapy may comprise an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. The cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. The cancer may be a Stage I cancer. The cancer may be a Stage II cancer. The cancer may be a Stage III cancer. The cancer may be a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Disclosed are methods for treating cancer originating from the colon. The cancer may be colon cancer. The cancer may be colorectal cancer.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Radiotherapy

Radiotherapy, such as ionizing radiation, may be administered to a subject. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

The radiotherapy can comprise external radiotherapy, internal radiotherapy, radioimmunotherapy, or intraoperative radiation therapy (IORT). The external radiotherapy may comprise three-dimensional conformal radiation therapy (3D-CRT), intensity modulated radiation therapy (IMRT), proton beam therapy, image-guided radiation therapy (IGRT), or stereotactic radiation therapy. The internal radiotherapy may comprise interstitial brachytherapy, intracavitary brachytherapy, or intraluminal radiation therapy. The radiotherapy may be administered to a primary tumor.

The amount of ionizing radiation is greater than 20 Gy and may be administered in one dose. The amount of ionizing radiation may be 18 Gy and is administered in three doses. The amount of ionizing radiation may be at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). The ionizing radiation may be administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of radiotherapy administered to a subject may be presented as a total dose of radiotherapy, which is then administered in fractionated doses. For example, the total dose may be 50 Gy administered in 10 fractionated doses of 5 Gy each. The total dose may be 50-90 Gy, administered in 20-60 fractionated doses of 2-3 Gy each. The total dose of radiation may be at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses may be administered (or any derivable range therein). At least, at most, or exactly 1, 2, 3, 4, 5, 6, 7, 8, 9, 10, 11, or 12 (or any derivable range therein) fractionated doses may be administered per day. At least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses may be administered per week.

### B. Cancer Immunotherapy

The methods may comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

The methods and compositions of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H₁, B7-4, CD274, and B7-H. Alternative names for “PDL2” include B7-DC, Btdc, and CD273. PD-1, PDL1, and PDL2 may be human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may be PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

The immune checkpoint inhibitor may be a PDL1 inhibitor such as Durvalumab, also known as MEDI4736, atezolizumab, also known as MPDL3280A, avelumab, also known as MSB00010118C, MDX-1105, BMS-936559, or combinations thereof. The immune checkpoint inhibitor may be a PDL2 inhibitor such as rHIgM12B7.

The inhibitor may comprise the heavy and light chain CDRs or VRs of nivolumab, pembrolizumab, or pidilizumab. The inhibitor may comprise the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may be one that blocks the CTLA-4 and B7-1 interaction. The inhibitor may be one that blocks the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

The inhibitor may comprise the heavy and light chain CDRs or VRs of tremelimumab or ipilimumab. The inhibitor may comprise the CDR1, CDR2, and CDR3 domains of the V_{H} region of tremelimumab or ipilimumab, and the CDR1, CDR2 and CDR3 domains of the V_{L} region of tremelimumab or ipilimumab. The antibody may be one that competes for binding with and/or binds to the same epitope on PD-1, B7-1, or B7-2 as the above-mentioned antibodies. The antibody may have at least about 70, 75, 80, 85, 90, 95, 97, or 99% (or any derivable range therein) variable region amino acid sequence identity with the above-mentioned antibodies.

c. Lag3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

The immune checkpoint inhibitor may be an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

The inhibitor may comprise the heavy and light chain CDRs or VRs of an anti-LAG3 antibody. The inhibitor may comprise the CDR1, CDR2, and CDR3 domains of the V_{H} region of an anti-LAG3 antibody, and the CDR1, CDR2 and CDR3 domains of the V_{L} region of an anti-LAG3 antibody. The antibody may have at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

The immune checkpoint inhibitor may be an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

The inhibitor may comprise the heavy and light chain CDRs or VRs of an anti-TIM-3 antibody. The inhibitor may comprise the CDR1, CDR2, and CDR3 domains of the V_{H} region of an anti-TIM-3 antibody, and the CDR1, CDR2 and CDR3 domains of the V_{L} region of an anti-TIM-3 antibody. The antibody may have at least about 70, 75, 80, 85, 90, 95, 97, or 99% (or any derivable range or value therein) variable region amino acid sequence identity with the above-mentioned antibodies.

### 2. Activation of Co-Stimulatory Molecules

The immunotherapy may comprise an activator of a co-stimulatory molecule. The activator may comprise an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Methods and compositions of the disclosure include patients that have been previously treated for a therapy described herein, are currently being treated for a therapy described herein, or have not been treated for a therapy described herein. The patient may be one that has been determined to be resistant to a therapy described herein. The patient may be one that has been determined to be sensitive to a therapy described herein. For example, the patient may be one that has been determined to be sensitive to an immune checkpoint inhibitor therapy based on a determination that the patient has or previously had pancreatitis.

### C. Chemotherapies

The additional therapy may comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be used as a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### D. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### E. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### F. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### XII. IMMUNOSTIMULATORS

Certain aspects of the present disclosure are directed to immunostimulators and methods of use. The term “immunostimulator” as used herein refers to a compound that can stimulate an immune response in a subject, and may include an adjuvant.

In some aspects, an immunostimulator is an agent that does not constitute a specific antigen, but can boost the strength and longevity of an immune response to an antigen. Such immunostimulators may include, but are not limited to stimulators of pattern recognition receptors (such as Toll-like receptors, RIG-1 and NOD-like receptors) mineral salts, such as alum, alum combined with monphosphoryl lipid (MPL) A of Enterobacteria, such as Escherihia coli, Salmonella minnesota, Salmonella typhimurium, or Shigella flexneri or specifically with MPL (AS04), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as ASO1, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments. Example immunostimulators include polymeric immunomodulators described herein.

### XIII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Synthesis and Use of Polymeric TLR 7/8 Modulator Adjuvant for Immune Activation and Vaccination Enhancement

An oligomeric peptide with repeating TLR7/8a (Imidazoquinoline) and dopamine units was generated (“pTLR7/8a_mod” or “p(TLR7/8a_dopa)”; see FIG. 7 for general structure and FIGS. 8A-8B for an overview of the synthesis scheme and FIG. 9 for the chemical structure). p(TLR7/8a_dopa) was analyzed in various in vitro and in vivo assays to evaluate immune activation and enhancement of vaccine efficacy.

### A. Materials and Methods

Peptide: peptides and modified peptides were synthesized using microwave Liberty blue peptide synthesizer using standard solid-phase peptide synthesis and purified (>90%) by high-performance liquid chromatography.

TLR-7/8 agonists: Imidazoquinoline-based TLR-7/8 agonists were produced as previously described (see J. Med. Chem. 2010, 53, 11, 4450-4465, incorporated herein by reference in its entirety).

pTLR7/8a: Imidazoquinoline-based TLR7/8a was modified with a C6 linker using NHS ester chemistry. The agonist was then loaded onto a Glutamic acid pentamer using HATU coupling chemistry. This peptide was modified with a DBCO handle.

pdopa: Fmoc-DOPA(acetonide)-OH was purchased form Sigma to make the pentamer of dopa using solid state peptide synthesis. This peptide was modified with an azide handle.

p(TLR7/8a_dopa): pTLR7/8a and pdopa were dissolved in DMF and mixed in a tube. The tube was shaken for 1 h at room temperature. The final product was purified using reverse phase HPLC.

BMDC activation: BMDCs were prepared from C57Bl/6 mice as previously described (see Chem. Sci., 2021, December, 6646-6651, incorporated herein by reference in its entirety) and used on day 7. For BMDC activation studies, 2×105 cells per well were seeded in round-bottom 96-well plates (Fisher Scientific) in RPMI with 10% FBS and 2% penicillin/streptomycin (Life Technologies), and treated with varying concentrations of mTLR7/8a (Imidazoquinoline), mTLR7/8a and dopamine mixture, or p(TLR7/8a_dopa), then incubated at 37° C. After 24 h, the samples were collected, and cytokine concentration was measured in the media by ELISA as detailed in the manufacturer's instructions.

RAW264.7 Macrophage (RAW-Blue) NF-κB assay: RAW-Blue cells (passage 5-15) were plated in a 96 well plate at a density of 100,000 cells/well in 180 μL DMEM containing 10% heat-inactivated FBS (HI-FBS) and selective antibiotics. The cells were treated with agonists for 20 h at 37° C. and 5% CO₂. NF-κB activity was measured by a QUANTI-Blue (InvivoGen) assay and the absorbance was measured at 620 nm using a Multiskan FC plate reader (Thermo Scientific).

Flow Cytometry for Cell Surface Marker Upregulation: BMDCs were plated in untreated 12-well plates at 1×106 cells/mL and incubated with agonists in culture media for 18 h at 37° C. with 5% CO₂. The cells were released from the plate by pipetting vigorously and centrifuged at 2500 RPM at 4° C. for 10 min. The cell pellet was resuspended in cold FACS buffer (PBS, 10% FBS, and 0.1% sodium azide) buffer (300 μL) and incubated with CD16/32 FcR blocking antibodies (1.0 μg/1×106 cells) on ice for 15 min. The cell suspension was pelleted, and the supernatant was removed. Next, the cell pellet was resuspended in cold FACS buffer (100 μL) and stained for CD86 and CD40 in the dark for 30 min. The samples were then washed twice with 300 μL FACS buffer. The pelleted cells were resuspended in cold FACS buffer (200 μL) and kept on ice until being loaded onto the flow cytometer for analysis.

Immunization studies with OVA: C57Bl/6 mice were vaccinated via s.c injection near the tail on days 0 and 14 with 100 μg of OVA formulated as OVA/p(TLR7/8a_dopa)/Alum, OVA/Alum, OVA/mTLR7/8a/Alum, OVA/(dopa+mTLR7/8)/Alum. Formulations containing TLR7/8 ligand contained molar equivalents of TLR7/8 ligand in monomeric TLR7 or polymeric form (25 nmol/mouse). On day 21 blood was collected and serum was separated by centrifugation and stored at −20° C. Sera were assayed for antibody levels against OVA using ELISA. The dLNs of each animal were collected and processed. Re-stimulation was carried out on lymph node cells over 2 days for the measurement of secreted cytokines by cytokine bead array (CBA).

### B. Results

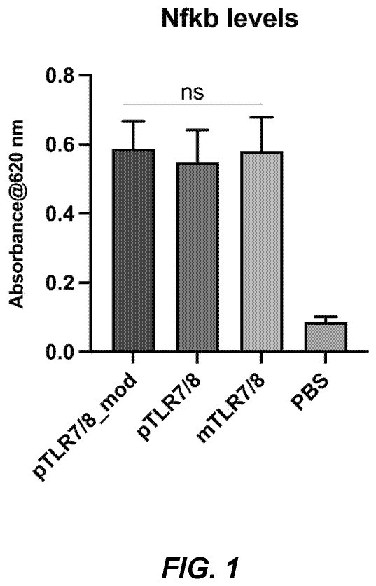

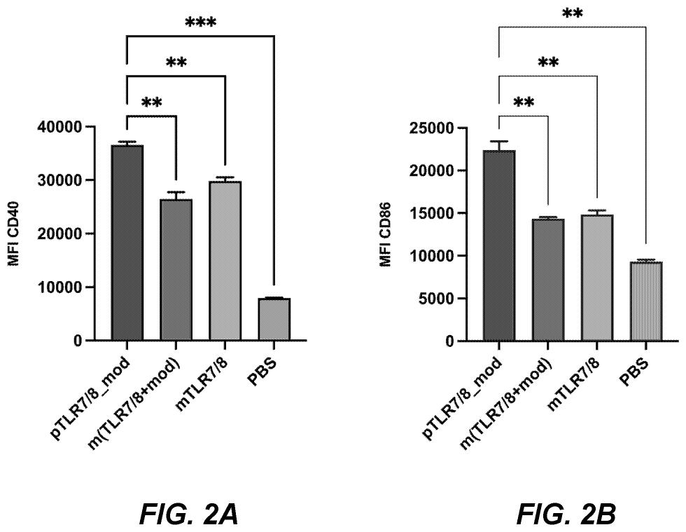

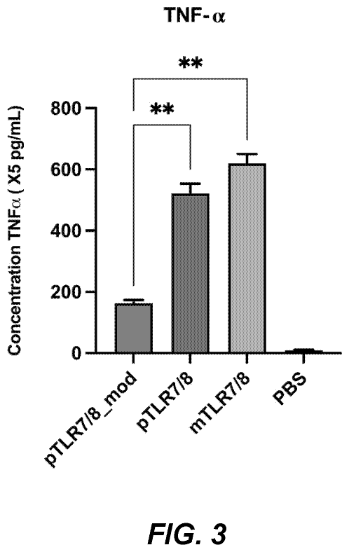

Bone marrow derived macrophages (BMDMs) were treated with pTLR7/8_mod, pTLR7/8, mTLR7/8, or phosphate buffered saline (PBS). FIG. 1 shows NF-κB levels, which were increased in response to treatment. FIGS. 2A and 2B show levels of CD40 (FIG. 2A) and CD86 (FIG. 2B). Treatment with pTLR7/8_mod showed the greatest increase in levels of both molecules. FIG. 3 shows TNF-α concentration measured in the supernatant of treated BMDMs. Treatment with pTLR7/8_mod showed the smallest increase in TNF-α levels compared with pTLR7/8 and mTLR7/8.

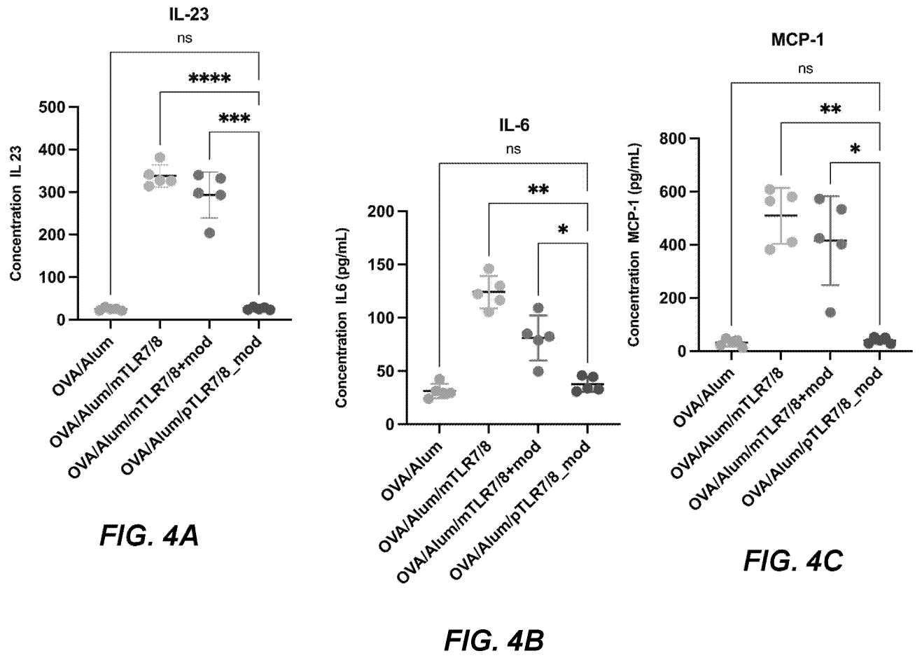

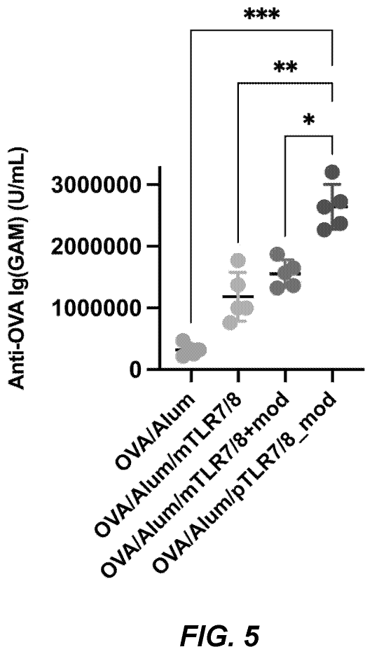

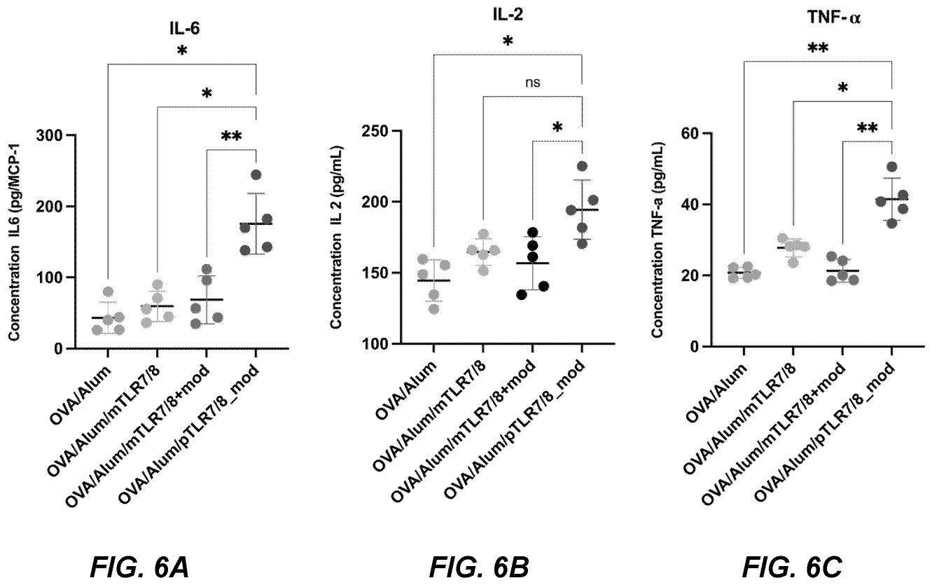

C57Bl/6 mice were vaccinated with ovalbumin antigen (“OVA”) with just alum or also in combination with pTLR7/8_mod, mTLR7/8, or mTLR7/8+dopamine (mTLR7/8+mod). FIGS. 4A-4C show serum concentration of IL-23 (FIG. 4A), IL-6 (FIG. 4B), and MCP-1 (FIG. 4C). Vaccination with pTLR7/8_mod shows the lowest levels of all three cytokines. FIG. 5 shows serum titers of anti-OVA antibodies in the mice. Highest anti-OVA antibodies were measured in the mice vaccinated with pTLR7/8_mod. FIGS. 6A-6C show amount of IL-23 (FIG. 6A), IL-6 (FIG. 6B), and TNF-α (FIG. 6C) produced by cells isolated from the draining lymph nodes of mice and re-stimulated with OVA for 48 hours. Cells from mice vaccinated with pTLR7/8_mod showed the highest levels of all three cytokines.

### Example 2—General Materials and Methods

Reagents and solvents were purchased from commercial sources and used without further purification. Vanillin, dopamine hydrochloride, 4-hydroxy-3-methoxybenzylamine 25 hydrochloride and ferulic acid were commercially obtained. APC anti-mouse CD40, PE anti-mouse CD11c and purified anti-mouse CD16/32 were purchased from BioLegend. COX-2 (mouse) Polyclonal Antibody was purchased from Cayman Chemical. GAPDH (14C10) Rabbit mAb and iNOS (D6B6S) Rabbit mAb were purchased from Cell Signaling Technology. Spectroscopic characterization was done on Bruker Avance III HD 500 11.7 Tesla NMR (500 MHz) for ¹H and ¹³C NMR. NMR spectra were analyzed using MestreNova software. Coupling on the spectra is expressed in hertz and abbreviations for multiplicities given as s=singlet, d=doublet, t=triplet, dd=doublet of doublets, and m=multiplet where applicable. Mass spectral analysis was performed on Agilent 6224 TOF-MS. Preparative reversed-phase HPLC purification was carried using Phenomenex Luna C₁₈ or C₈ Prep (150×21.2250 mm, 5 μm particle size) column with a flow rate of 21.2 mL/min on a Gilson 333/334 pump system and GX-271 liquid handler system. UV detection (214 nm, 254 nm, and 260 nm) was used for preparative HPLC. Flow Cytometry data was acquired on a NovoCyte Benchtop Flow Cytometer. Absorbance measurements were acquired on a Multiskan FC plate reader (Thermo Scientific). Data was analyzed using one-way ANOVA in Graph Pad Prism software. All values were reported as mean±SD. Female C57/BL6 mice and male Balb/c mice were purchased from Jackson Laboratories and allowed to equilibrate for a minimum of 48 h before use. For all experiments, the mice were 6-10 wk old. All animal studies and mice maintenance were approved by the Institutional of Animal Care and Use (IACUC #2012-3048).

### Example 3—RAW264.7 Macrophage (RAW-Blue) NF-κB Assay

RAW-Blue cells, (InvivoGen) were cultured as described by the manufacturer. Cells were grown in complete culture media composed of Dulbecco's Modified Eagle's Medium (DMEM) with 4.5 g/L glucose (Life Technologies), 2 mM L-glutamine, 10,000 U/mL penicillin, 10 mg/mL streptomycin, 25 g/mL amphotericin B, supplemented with 10% fetal bovine serum (FBS, Thermo Fisher Scientific). RAW-Blue cells (passage 5-15) were plated in a 96 well plate at a density of 100,000 cells/well in 180 μL DMEM containing 10% heat-inactivated FBS (HI-FBS) and selective antibiotics. The cells were treated with agonist and agonist dimers and LPS control (50 ng/mL) for 20 h at 37° C. and 5% CO2. NF-κB activity was measured by a QUANTI-Blue (InvivoGen) assay and the absorbance was measured at 620 nm using a Multiskan FC plate reader (Thermo Scientific).

### Example 4—Bone Marrow-Derived Dendritic Cell Harvest and Culture

Bone marrow-derived dendritic cells (BMDCs) were harvested from female C57Bl/6 mice. Femur bones were aseptically removed from mice and the bone marrow was extracted into PBS buffer and the cell suspension centrifuged at 300 RCF for 10 min at RT to pellet the cells. ACK Lysing Buffer (3 mL, Lonza) was added to the cell pellet and incubated for 2 min at room temperature (RT). PBS buffer (13 mL) was then added to the cell suspension, and the cell solution was centrifuged at 300 RCF for 10 min at RT. Next, the cell pellet was resuspended in BMDC complete media (RPMI 1640, 10% heat-inactivated FBS, 20 ng/mL granulocyte-macrophage colony-stimulating factor (GM-CSF), 2 mM L-glutamine (Life Technologies), 10,000 U/mL penicillin, 10 mg/mL streptomycin, 25 μg/mL amphotericin B, and 50 μM beta-mercaptoethanol). The cells were then plated at 1×10⁶ cells/mL in 100 mm petri dishes in 10 mL complete media and incubated at 37° C. in a CO₂ incubator. On day 3, 10 mL of fresh BMDC media was added to each petri dish. On day 6, BMDCs were released and plated in untreated 12-well plates at 1×10⁶ cells/mL for cell surface marker activation and cytokine secretion experiments.

### Example 5—Western Blot Analysis of Pathway Proteins

RAW264.7 Macrophage cells (InvivoGen) cultured in complete media were plated in 6 well plates at 1×10⁶ cells/mL and allowed to adhere for 12 h at 37° C. in a CO₂ incubator. The cells were then treated with agonist, agonist dimers, and LPS control for 16 h. The treated cells were washed and scraped into cold phosphate-buffered saline (PBS) and centrifuged at 400×g at 4° C. for 5 min. The cell pellets were resuspended in triple detergent lysis buffer (10 mL) containing one protease inhibitor cocktail (cOmplete™ ULTRA Tablets, Sigma) and centrifuged to yield whole cell lysate. The lysate was quantified using a Pierce™ BCA Protein Assay Kit. 50 μg of total protein was separated using 4-15% SDS-PAGE and blotted onto PVDF membranes (Bio-Rad). The membranes were probed using monoclonal antibodies for COX-2 at a dilution of 1:1000 (Cayman Chemicals, MI) GAPDH (14C₁₀) at a dilution of 1:1000 and Rabbit mAb and at a dilution of 1:500 iNOS (D6B6S) Rabbit mAb Visualization was achieved using IRDye® 800CW (Abcam) at a dilution of 1:10000 and imaged on Azure biosystems imager. Densitometric analysis was done using Image J.

### Example 6—Flow Cytometry for Cell Surface Marker Upregulation and Cytokine Secretion Analysis

BMDCs were plated in untreated 12-well plates at 1×10⁶ cells/mL and incubated with agonist and agonist dimers in culture media for 8 h at 37° C. with 5% CO₂. The cells were released from the plate by pipetting vigorously and centrifuged at 2500 RPM at 4° C. for 10 min. The cell culture media was saved for IL-6 cytokine quantification using ELISA (BioLegend). The cell pellet was resuspended in cold FACS buffer (PBS, 10% FBS, and 0.1% sodium azide) buffer (300 μL) and incubated with CD16/32 FcR blocking antibodies (1.0 μg/1×106 cells) on ice for 15 min. The cell suspension was pelleted, and the supernatant was removed. Next, the cell pellet was resuspended in cold FACS buffer (100 μL) and incubated with PE-CD11c (1.0 μg/1×10⁶ cells) and APC CD40 (1.0 μg/1×10⁶ cells), on ice and in the dark for 30 min. The samples were then washed twice with 300 μL FACS buffer. The pelleted cells were resuspended in cold FACS buffer (200 μL) and kept on ice until being loaded onto the flow cytometer for analysis.

### Example 7—In Vivo Vaccination of Mice

Female C57/BL6 mice were briefly anesthetized with isoflurane and injected intramuscularly in the right hind leg with 50 μL containing ovalbumin (100 μg), adjuvant, adjuvant dimers, vanillin, and dopamine (0.07 μmoles) and a PBS vehicle control group.

### Example 8—Plasma Cytokine Analysis and Antibody Quantification

Mouse blood was collected via the submandibular vein in 0.2 mL heparin-coated collection tubes (VWR Scientific) 1 h after vaccination. Serum was isolated by allowing blood to clot for 30 min RT and centrifugation at 2000×g for 10 min. Supernatant was collected and stored at −80° C. until use. Serum was analyzed using BD Cytometric Bead Array Mouse Inflammation cytokine kit or LEGENDplex™ Mouse Inflammation Panel (Biolegend) according to manufacturer's protocol. For antibody quantification, mouse blood was collected via cardiac puncture 28 days after vaccination in 0.2 mL heparin-coated collection tubes (VWR Scientific). Serum was isolated by allowing blood to clot for 30 min RT and centrifuging at 2000×g for 10 min. Serum was analyzed using a quantitative anti-ovalbumin total Ig's, IgA, and IgG ELISA kits (Alpha Diagnostic International) according to the manufacture's protocol.

### Example 9—Tumor Studies

0.2×10⁶ CT-26 cells were injected subcutaneously into the flank of 6-week-old Balb/c mice (n=10 per group) in 100 μL of PBS. The tumor size was monitored on alternating days. Tumor volumes were measured using the equation V=1/2×L×W×W. When the tumors reached a size of approximately 75 mm3 (day 11), treatment was started. Various formulations (20 nmols of each agonist or PBS) were injected peritumorally every 4 days (day 15, 19, and 23). Mice were euthanized when the tumors reached 20 mm in any linear dimension. Five mice for each group were used for blood analysis. Blood was collected two days post the first injection for hematological toxicity analysis and two hours post the second injection for systemic cytokine analysis.

### Example 10—Nitric Oxide Assay

RAW264.7 Macrophage (InvivoGen) cultured in complete media were plated in 12-well plates at 1×10⁶ cells/mL and treated with agonist and agonist dimers for 16 h at 37° C. in a CO₂ incubator. The cell supernatant was collected and the quantity of nitrite in the culture medium was measured as an indicator of NO production. Amounts of nitrite, a stable metabolite of NO, were measured using Griess reagent (1% sulfanilamide and 0.1% naphthyl ethylenediamine dihydrochloride in 2.5% phosphoric acid). 100 μL of cell culture medium was mixed with 100 μL of Griess reagent. After incubation at room temperature for 10 min, the absorbance at 540 nm was measured in a microplate reader. The quantity of nitrite was determined from a sodium nitrite standard curve.

### Example 11—Reactive Oxygen Species Measurement

RAW264.7 Macrophage (InvivoGen) cultured in complete media, were plated in 12-well plates at 1×10⁶ cells/mL and treated with agonist and agonist dimers for 16 h at 37° C. in a CO₂ incubator. The cells were released from the plate and centrifuged at 2500 RPM at room temperature for 10 min and the supernatant aspirated. The cells were then washed twice with PBS (200 μL) and the cell pellet resuspended in Hanks' Balanced Salt solution (HBSS) containing CM-H₂DCFDA (1 μM). Next, the cells were incubated for 30 min at 37° C. with 5% CO₂. After incubation, the cells were washed twice with cold PBS. Fluorescence was measure using Flow Cytometry on FL-1 (fitc) channel.

### Example 12—Proliferation Assay

Proliferation assay was performed as previously reported. Splenocytes were isolated from C57BL/6 mice and plated at 5×104 in 96-well plates. The splenocytes were then incubated with 3 μM of agonist and agonist dimers for 48 h at 37° C. in a CO₂ incubator. 3-[4,5-dimethylthiazol-2-yl]-2,5-diphenyltetrazolium bromide (MTT) (20 μl, 5 mg/ml in PBS) was added 4 h before the end of the incubation period. Purple crystals were dissolved in sterile DMSO and incubated for 5 minutes to ensure complete dissolution. The absorbance was measured at 590 nm using a Multiskan FC plate reader (Thermo Scientific). The proliferation rate was determined as follows: Abs(sample)/Abs(PBS)×100%.

### Example 13—Cell Viability (MTT) Assay

RAW264.7 Macrophage cells were incubated with agonist and agonist dimers for 16 h and subjected to cell viability assays. 3-(4,5-dimethylthiazol-2-yl)-2,5-diiphenyltetrazolium bromide (MTT) was dissolved in PBS to final concentration of 5 mg/mL and sterile-filtered. Treated cells were resuspended in fresh RPMI medium with 10% FBS at concentration and plated in a 96-well plate at a concentration of 1×105 cell/mL. To these cells, 10 μL of MTT solution was added, then incubated at 37° C. with 5% CO₂ for 3 h. When purple crystals were visible, 75 μL of supernatant was removed. Purple crystals were dissolved in sterile DMSO and incubated for 5 minutes to ensure complete dissolution. The absorbance was measured at 590 nm using a Multiskan FC plate reader (Thermo Scientific). % viability was calculated as follows: Abs(sample)−Abs(blank)/Abs(Rest cells)−Abs(blank)×100%.

### Example 14—Synthesis of Imidazoquinolinone-Immune Potentiator Dimer

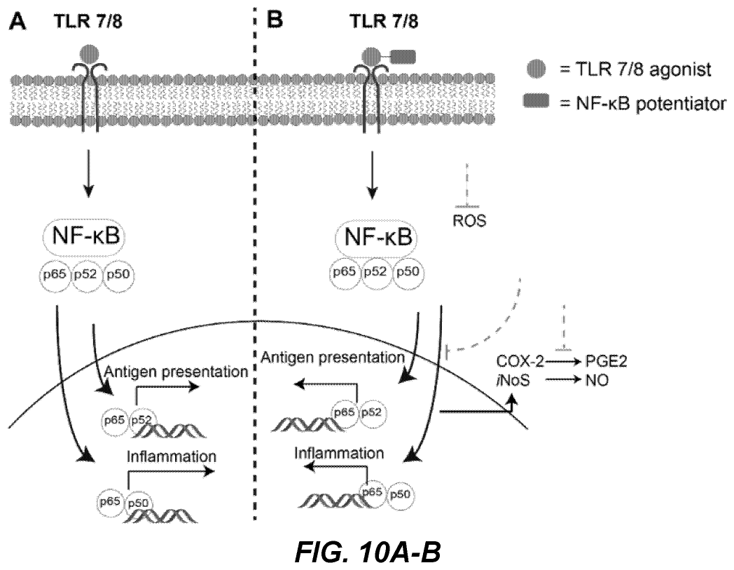

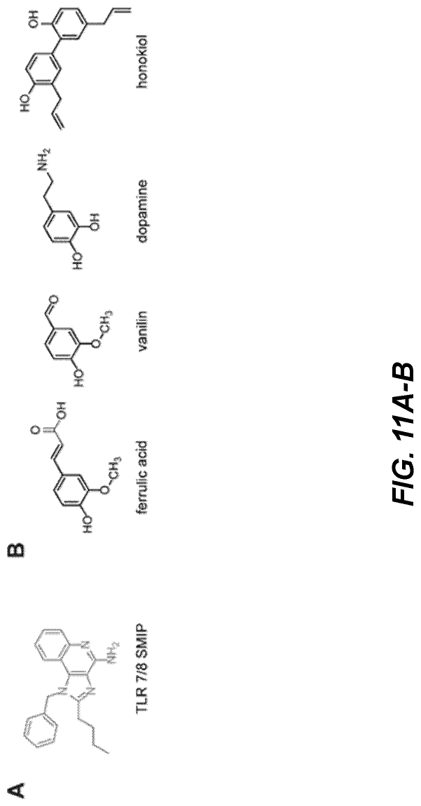

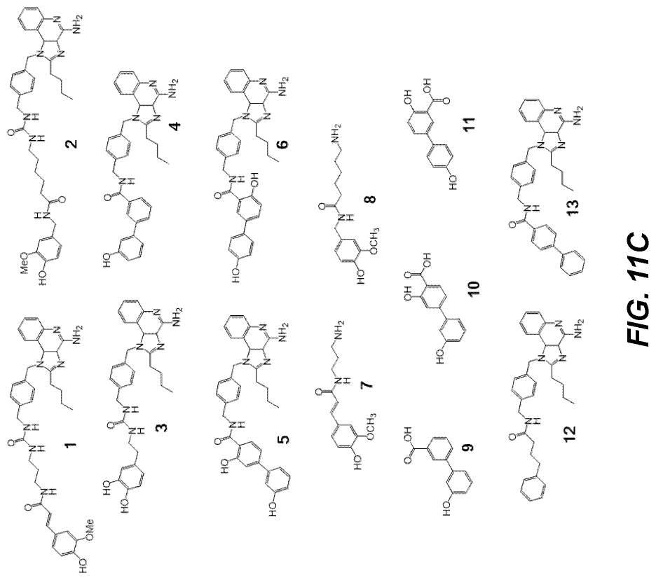

Various small molecule NF-κB inhibitors in vaccine formulations were screened both in vivo and in vitro. From this screen, vanillin and honokiol derivatives were identified as the most effective small molecule immune potentiators.¹⁹ These anti-inflammatory and antioxidant molecules have been extensively studied in literature for NF-κB modulation through direct inhibition of the canonical NF-κB pathway or through scavenging pro-inflammatory mediators such as nitric oxide and other ROIs.²¹⁻²³ When in vivo experiments were performed using a mixture of R848 and capsaicin or honokiol potentiators in vaccine formulations, high systemic cytokines were observed 1 hour after vaccination (FIGS. 10A-10B). A series of dimers were then designed and synthesized by conjugating a TLR 7/8 imidazoquinolinone derivative with a conjugatable amine handle²¹ to moieties based on vanillin, catechol and honokiol (FIG. 11).

### Example 15—In-Vitro Analyses of Synthesized Dimers

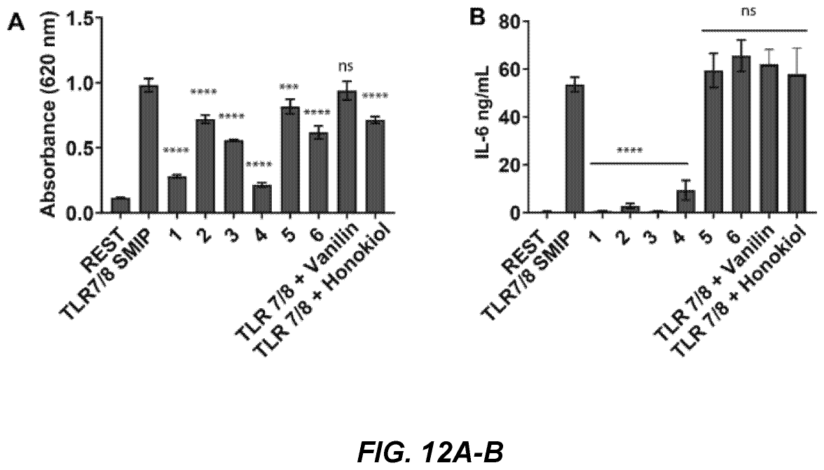

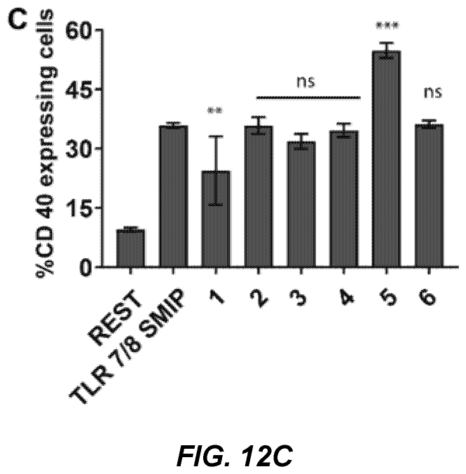

Next, an in vitro screen was designed to test the synthesized dimers and identify promising candidates for further development in an in vivo model. Using a RAW macrophage NF—KB-SEAP reporter cell line, the overall activities of the compounds was measured. A reduction in activity of the dimers was observed compared to the imidazoquinolinone agonist and equimolar mixtures of the agonist and NF-κB inhibitors (FIG. 12A). However, it was possible that the reduction in activity could be attributed to the conjugation of the molecules lowering the activity of the parent compound by disrupting cellular uptake or receptor binding. To further elucidate the activity of the dimers, pro-inflammatory cytokine and cell surface protein expression assays were performed on murine bone marrow derived macrophages (BMDCs). After incubating the parent SMIP and the dimers with BMDCs for 8 h, dimer compounds 1-4 reduced the levels of IL-6 secreted to almost baseline levels. Compounds 5 and 6 did not significantly change the IL-6 levels when compared to the parent SMIP. Equimolar mixture of the SMIP and the small molecule NF-κB inhibitors also did not lower the levels of IL-6 secreted (FIG. 12B). In a similar BMDC experiment the BMDCs were stained for cell surface expression of CD40, a well characterized co-stimulatory molecule with an important role in adaptive immunity,²⁴ and the expression levels were quantified using cytometry. In this experiment, the expression levels of CD40 remained unchanged for most of the compounds and the expression levels were slightly lower for compound 1 (FIG. 12C). This experiment was used to screen for dimers that would lower pro-inflammatory cytokines while maintaining or improving cell surface protein expression. This would indicate that the hybrid molecule was modulating the NF-κB response of the imidazoquinolinone as opposed to merely inhibiting the activity.

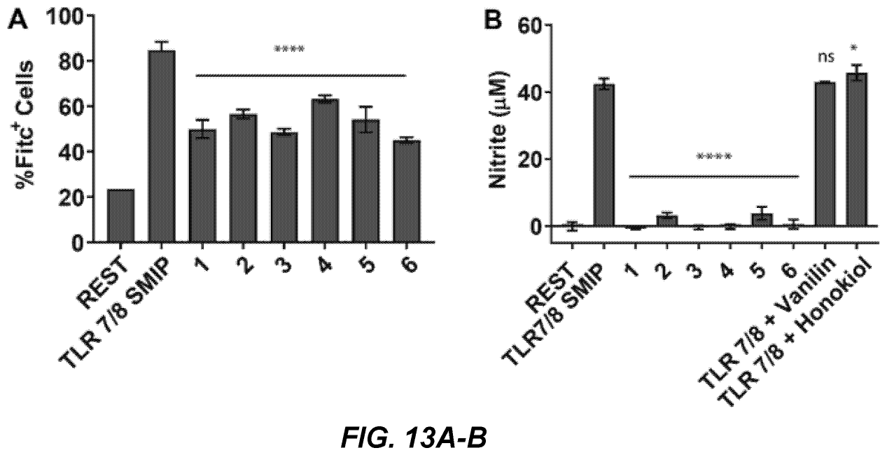

In addition to inhibition of the upstream events of NF-κB pathway, the small molecule NF-κB inhibitors that were incorporated into the adjuvant dimers had previously been described in literature as downstream inhibitors of pro-inflammatory mediators such as nitric oxide and reactive oxygen species (ROS).²³ The dimer adjuvants were then examined for similar effects on immune cells. To study the effect of the dimers on oxidative stress, RAW macrophages were incubated with the compounds for 16 h and the levels of intracellular ROS were measured using ROS-reactive fluorescent dye, CM-H2DCFDA and quantified the fluorescence using Flow cytometry. Here, intracellular ROS was reduced by almost half for all the dimer agonists, suggesting that these compounds were ROS scavengers (FIG. 13A). In a similar experiment RAW macrophages were incubated for 16 h with the dimer agonists and Griess reagent was used to measure the levels of nitrite, a metabolite of nitric oxide in the cell supernatant. The nitrite levels were quantified by measuring absorbance using a plate reader. This analysis showed that the dimer adjuvants reduced NO levels to baseline levels compared to the parent SMIP (FIG. 13B), indicating that the dimers were potent inhibitors of nitric oxide either through scavenging or inhibition of inducible nitric oxide synthase (iNOS).

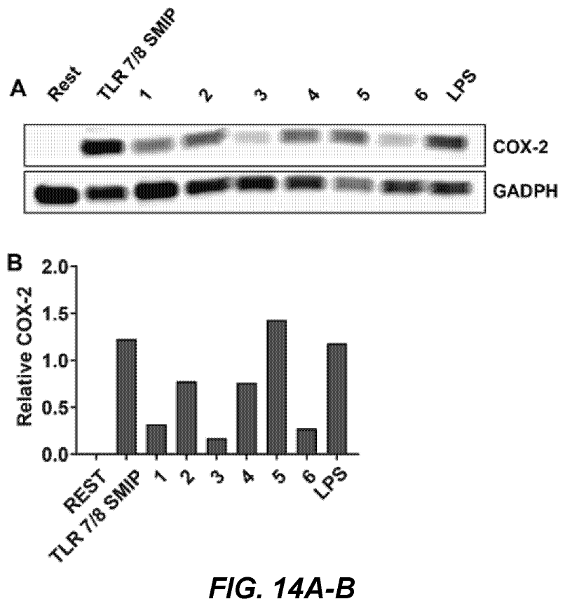

Lastly, dimer agonists' effects on inhibition of Cyclooxygenase-2 (COX-2) was examined. COX-2 is a pro-inflammatory marker associated with the activation of both the NF-κB and MAPK pathways. After incubating RAW macrophages with the dimer agonists for 16 h, the cells were lyzed and the proteins were separated using SDS-PAGE. The proteins were then transferred to a membrane and the protein levels were probed using an anti-COX-2 antibody. Low relative expression of COX-2 protein was observed with compounds 1-4 and 6 suggesting that these compounds were slightly inhibiting the COX-2 pathway (FIGS. 14A-14B).

### Example 16—In-Vivo Analysis

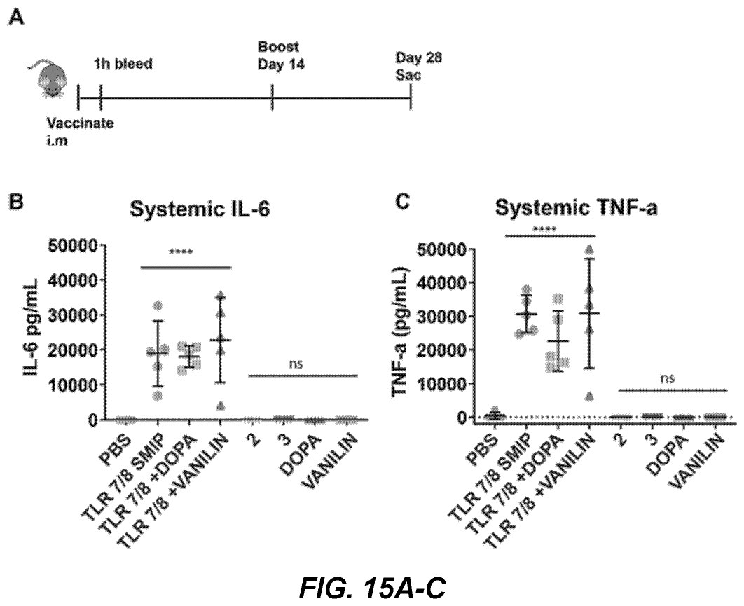

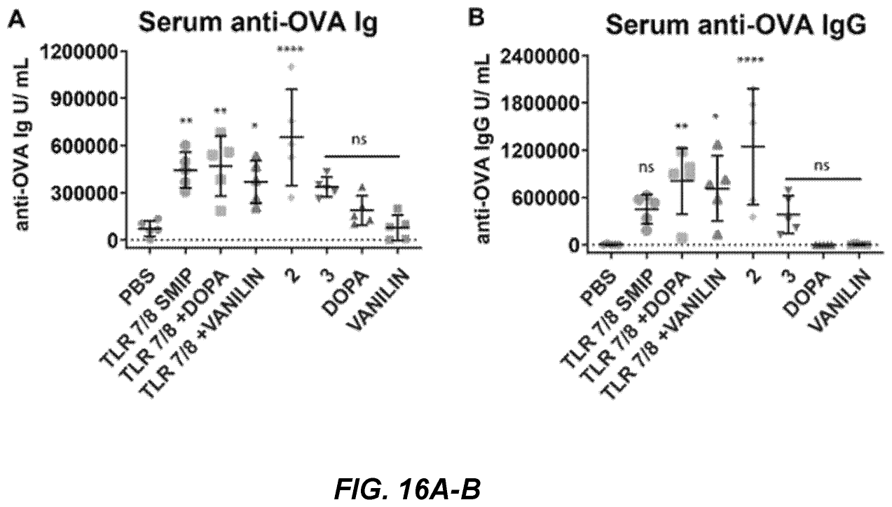

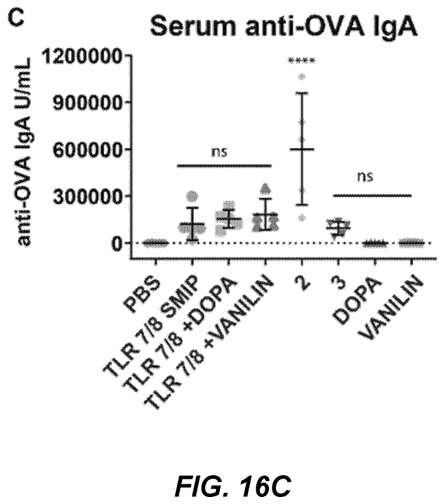

Next, in vivo studies were performed to see how these dimers would perform in a vaccine formulation. An outline of these studies is depicted in FIG. 15A. Using ovalbumin as a model, antigen mice were vaccinated with the most promising dimers (2, 3) from the in vitro screen. Intramuscular injections (i.m) were performed with 100 μg of ova, 70 nmoles of imidazoquinolinone, dimers, and equimolar mixtures of imidazoquinolinone and NF-κB inhibitors in 50 μl of PBS. At the 1-hour mark post injection, serum was collected from the mice and systemic levels of IL-6 (FIG. 15B) and TNF-α (FIG. 15C) were quantified. The dimers reduced these systemic cytokines to background levels comparable to the PBS, vanillin, and catechol controls. On day 28, the mice were sacrificed, sera collected, and anti-OVA antibodies were analyzed (FIGS. 16A-16C). Looking at total Ig levels, comparable levels across the TLR7/8 adjuvanted mice were observed compared to PBS and vanillin and catechol controls. Comparing specific anti-OVA IgG (FIG. 16B) and IgA antibodies (FIG. 16C), compound 2 induced statistically higher levels of these antibodies.

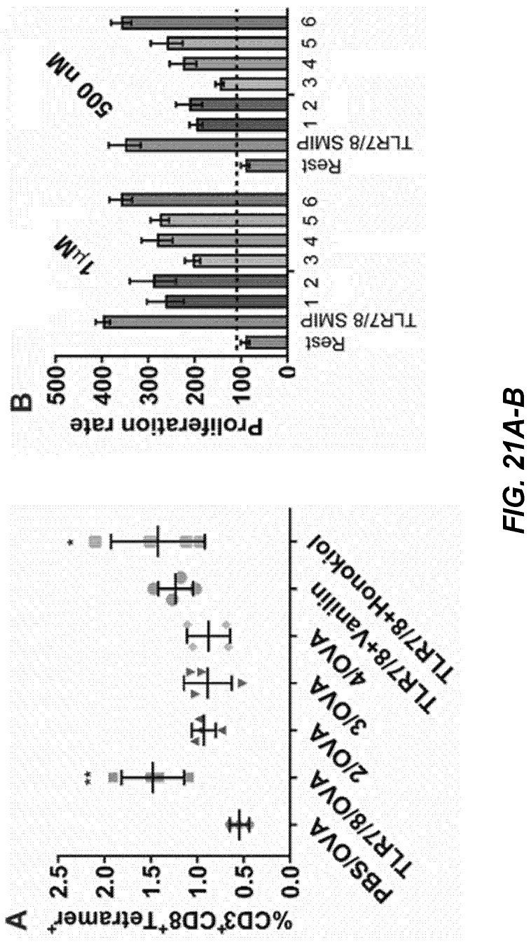

Because activation of TLR 7/8 has been previously associated with increased CD8+ T cell function,¹⁷ the activity of dimer adjuvants was investigated and measured to the activity of the parent SMIP adjuvant. On day 28 of the OVA in vivo vaccination experiment, the spleen of the mice were harvested and a single cell spleenocyte suspension was prepared. The cells were then incubated with a SINFEKL MHC specific tetramer and analyzed the cells using Flow cytometry. Increased activity was not observed with the agonist dimers compared to the vehicle control. The parent SMIP adjuvant was also only slightly higher than the PBS/OVA control (FIG. 21A). This was not surprising because this route of administration, admixing OVA and the adjuvant in PBS and i.m injection, has been shown by others to enhanced antibody titers but not T-cell immunity. In a separate analysis, spleenocyte proliferation was examined with naïve spleenocytes, incubating the cells for 48 h with the dimer agonists and parent SMIP. The dimer agonists promoted the proliferation of lymphocytes to comparable levels when compared to the parent SMIP (FIG. 21B).

### Example 17—Local Anti-Tumor Activity of Dimer Agonists

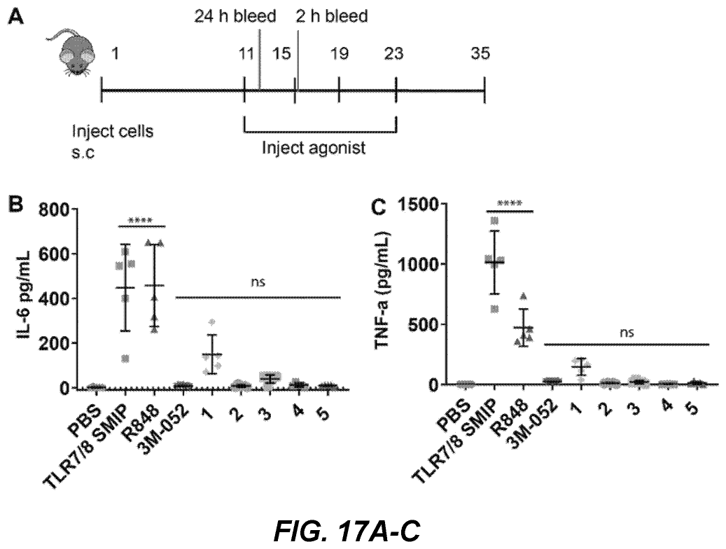

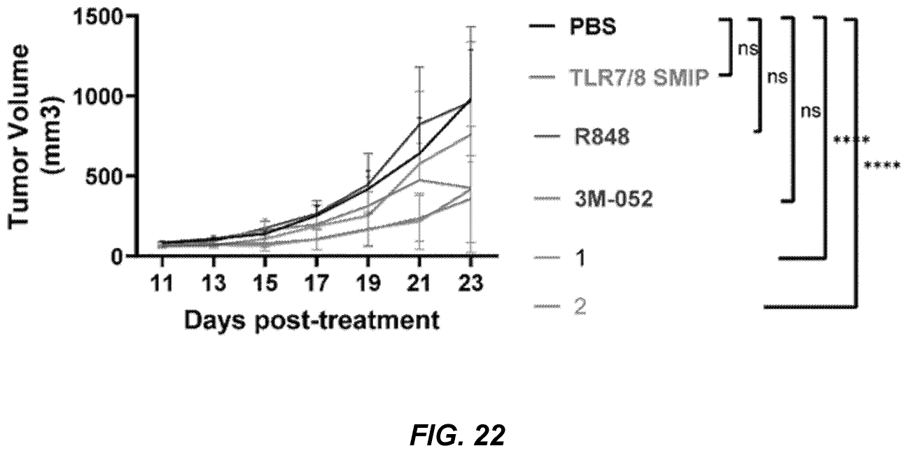

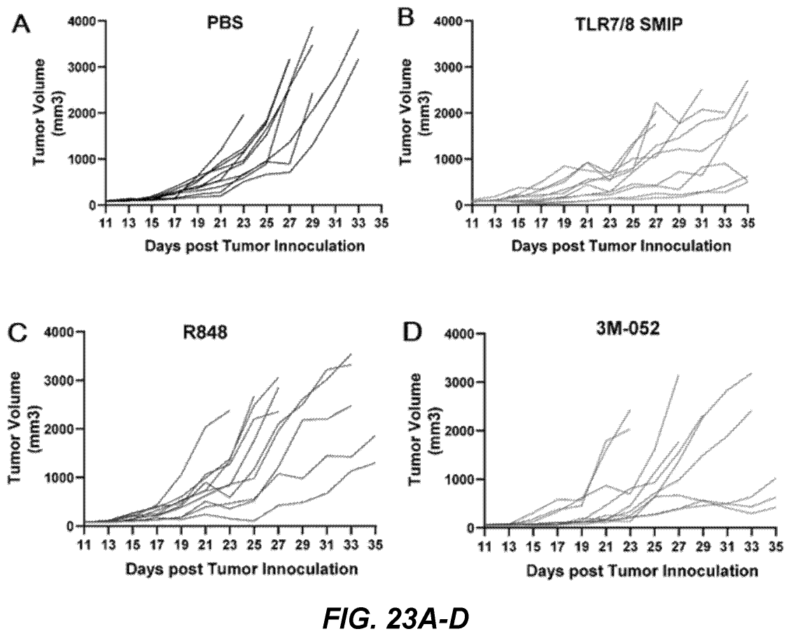

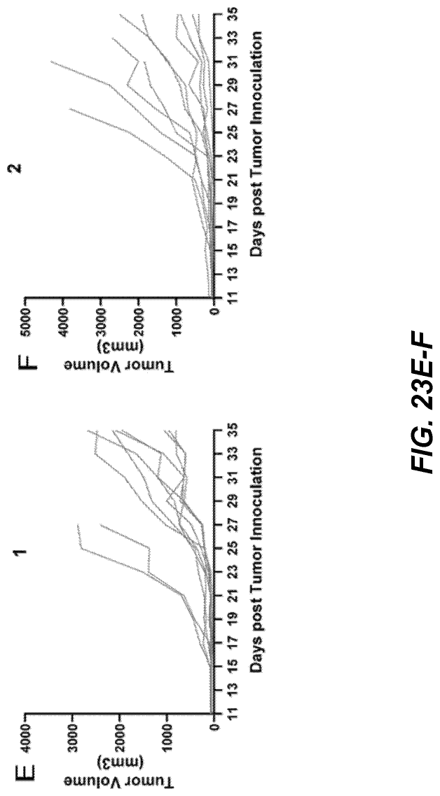

The anti-tumorigenic activity of the dimers was then examined. Previous studies have shown that intratumoral adjuvant introduction was effective in reducing tumor proliferation by enhancing T-cell anti-tumor activity. Specifically, TLR 7/8 activation leads to enhanced innate immune cell activation propagated by increased secretion of IFNα, IL-12 and IFNγ cytokines.^{17,25} For most compounds used for this purpose, e.g., resiquomot (R848), systemic adjuvant toxicity is always a drawback. As the present dimer agonist platform reduced systemic cytokines, the compounds were then tested on a tumor model (FIGS. 17A, 22, and 23A-23F). In this in vivo model, the CT26 tumor models were used and adjuvants, a PBS control, and adjuvant dimers were administered. Additionally, resiqumod (R848) along with a resiquimod derivative 3M-052 were included as controls. 3M-502 has been used in some clinical studies due to its ability to be formulated in liposomes or emulsions which reduces the diffusion rate due to the formation of a depot.²⁵ In this study however, all adjuvants were formulated in PBS.

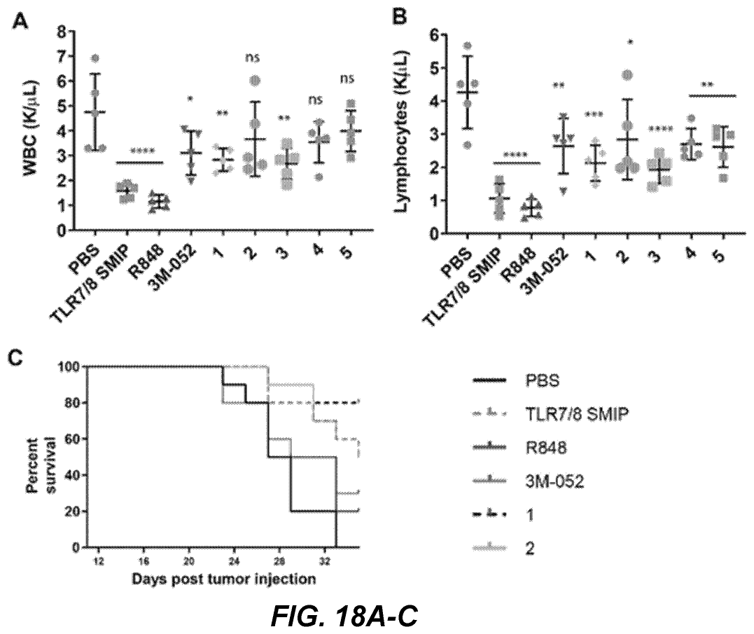

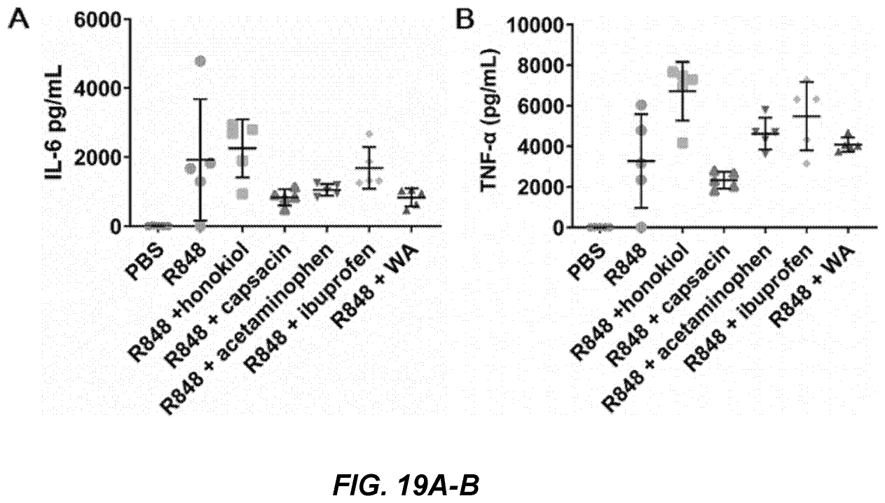

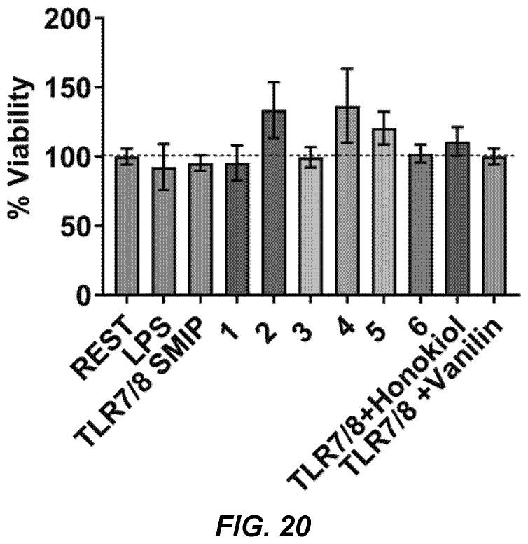

After 11 days, the tumors were established and intratumoral injections were administered with the SMIP adjuvant and adjuvant dimers. At 2 h and 24 h after these injections, serum and blood were collected to examine systemic cytokines in the serum and perform a hematological analysis on the blood to better quantify adjuvant toxicity. Similar to the OVA vaccination model, the dimer adjuvants induced baseline levels of IL-6 and TNF-α as measured in the serum. By contrast, the parent SMIP and R848 induced high levels of these pro-inflammatory cytokines, while 3M-052 was comparable to the the vehicle control and the dimer adjuvants. Interestingly, compound 1 induced slightly elevated levels of IL-6 (FIG. 17B) and TNF-α (FIG. 17C) when compared to the rest of the tested dimer adjuvants. Additionally, the hematological analysis of the blood showed that the molecules that induced higher systemic inflammatory cytokines led to lower WBC (FIG. 18A) and lymphocyte (FIG. 18B) counts compared to the vehicle control, suggesting that these molecules were causing adjuvant toxicity when introduced intratumorally.

Observing promising tolerability with the adjuvant dimers, tumor proliferation was then monitored (FIG. 18C). After 32 days, all the vehicle-treated mice had to be euthanized due to tumor proliferation. Of the dimer adjuvants tested, two dimer molecules 1 and 2 were able to improve survivability with compound 1 providing 80% survivability at day 35, which was higher than the parent adjuvant, 3M-052 and R848. Slightly elevated levels of IL-6 and TNF-α were observed in the serum of mice injected with compound 1, suggesting a possible need for localized inflammation for successful immunotherapy adjuvant candidates. Immune checkpoint inhibitors may be included in the dimer adjuvant formulation to bolster the dimers, thereby making the therapy effective as well as tolerable.

### Example 18—Synthetic Methods

Compound 1 (FIG. 11C) was synthesized as follows. To a solution of 7 (19 mg, 0.078 mmol) and triethylamine (0.01 mL, 0.078 mmol) was added 12 (26 mg, 0.052 mmol) in 1 mL DMF. The mixture was stirred at room temperature under argon for 12 h. The reaction mixture purified using preparative HPLC to give the product as a white solid (15 mg, 40% yield) ¹H NMR (400 MHz, DMSO) δ 13.47 (s, 1H), 7.95 (t, J=8.3 Hz, 2H), 7.80 (d, J=8.0 Hz, 1H), 7.62 (t, J=7.4 Hz, 1H), 7.38 (t, J=7.3 Hz, 1H), 7.30 (d, J=15.7 Hz, 1H), 7.21 (d, J=8.2 Hz, 2H), 7.12 (d, J=1.7 Hz, 1H), 7.02 (d, J=8.1 Hz, 2H), 6.98 (dd, J=8.2, 1.8 Hz, 1H), 6.79 (d, J=8.1 Hz, 1H), 6.42 (d, J=15.7 Hz, 1H), 5.93 (s, 2H), 4.15 (s, 2H), 3.80 (s, 4H), 3.14 (dd, J=12.6, 6.5 Hz, 2H), 2.98 (dd, J=16.6, 9.0 Hz, 4H), 1.73 (m, J=15.3, 7.6 Hz, 2H), 1.57-1.47 (m, 2H), 1.39 (m, J=14.7, 7.4 Hz, 2H), 0.87 (t, J=7.4 Hz, 3H). ¹³C NMR (100 MHz, DMSO) δ 165.8, 158.9, 158.6, 158.5, 157.4, 149.2, 148.7, 148.3, 141.2, 139.3, 135.8, 134.3, 134.1, 129.9, 128.0, 126.9, 125.9, 125.2, 125.1, 122.0, 119.4, 119.0, 116.0, 114.9, 112.9, 111.1, 55.9, 43.9, 36.7, 30.7, 29.7, 29.4, 26.6, 22.9, 22.2, 14.1. HRMS (ESI) m/z calculated for C₃₆H₄₁N₇O₄, [M+H]⁺ 635.322; found: 636.3294.

Compound 2 was synthesized as follows. To a solution of 8 (20 mg, 0.066 mmol) and triethylamine (0.01 mL, 0.078 mmol) was added 12 (26 mg, 0.052 mmol) in 1 mL DMF. The mixture was stirred at room temperature for 12 h. The reaction mixture purified using preparative HPLC to give the product as a white solid (18 mg, 54% yield) ¹H NMR (400 MHz, DMSO) δ 13.51 (s, 1H), 8.15 (t, J=5.8 Hz, 1H), 7.95 (d, J=8.2 Hz, 1H), 7.79 (d, J=8.0 Hz, 1H), 7.62 (t, J=7.4 Hz, 1H), 7.37 (t, J=7.4 Hz, 1H), 7.18 (t, J=7.3 Hz, 2H), 7.01 (d, J=8.1 Hz, 2H), 6.79 (d, J=1.7 Hz, 1H), 6.69 (d, J=8.0 Hz, 1H), 6.62 (dd, J=8.0, 1.8 Hz, 1H), 5.93 (s, 2H), 4.13 (d, J=5.5 Hz, 4H), 3.72 (s, 5H), 2.95 (dd, J=15.7, 8.0 Hz, 4H), 2.08 (dd, J=9.4, 5.3 Hz, 2H), 1.72 (m, J=15.3, 7.6 Hz, 2H), 1.49 (m, J=15.0, 7.5 Hz, 2H), 1.35 (m, J=22.4, 14.9, 7.2 Hz, 4H), 1.20 (m, J=14.7, 7.3 Hz, 2H), 0.87 (t, J=7.4 Hz, 3H). ¹³C NMR (100 MHz, DMSO) δ 172.3, 159.0, 158.7, 158.4, 157.4, 149.3, 147.9, 145.8, 141.2, 135.8, 134.4, 134.1, 130.9, 129.8, 128.0, 125.8, 125.2, 125.1, 122.0, 120.1, 118.9, 115.6, 112.9, 112.1, 55.9, 48.6, 42.9, 42.3, 35.8, 30.2, 29.7, 26.7, 26.5, 25.6, 22.2, 14.1. HRMS (ESI) m/z calculated for C₃₇H₄₅N₇O₄, [M+H]⁺ 651.3533; found: 652.3610.

Compound 3 was synthesized as follows. To a solution of dopamine hydrochloride (20 mg, 0.105 mmol) and triethylamine (0.027 mL, 0.105 mmol) was added 12 (26 mg, 0.052 mmol) in 1 mL DMF. The mixture was stirred at room temperature for 12 h. The reaction mixture purified using preparative HPLC to give the product as a white solid (9.3 mg, 33% yield) ¹H NMR (400 MHz, DMSO) δ 13.53 (s, 1H), 7.95 (d, J=8.3 Hz, 1H), 7.79 (d, J=8.0 Hz, 1H), 7.62 (t, J=7.8 Hz, 1H), 7.37 (t, J=7.4 Hz, 1H), 7.18 (t, J=6.6 Hz, 2H), 7.01 (d, J=8.1 Hz, 2H), 6.61 (d, J=7.9 Hz, 1H), 6.55 (d, J=1.9 Hz, 1H), 6.40 (dd, J=8.0, 1.9 Hz, 1H), 5.93 (s, 2H), 4.13 (s, 2H), 3.11 (t, J=7.0 Hz, 2H), 3.00-2.89 (m, 2H), 1.73 (m, J=15.3, 7.6 Hz, 2H), 1.37 (m, J=14.6, 7.4 Hz, 2H), 0.87 (t, J=7.3 Hz, 3H). ¹³C NMR (100 MHz, DMSO) δ 159.1, 158.7, 158.3, 157.4, 149.3, 145.5, 143.9, 141.1, 136.3, 135.8, 134.3, 134.06, 132.28, 130.83, 129.89, 128.06, 125.90, 125.23, 125.13, 122.96, 122.04, 119.67, 118.96, 117.83, 116.4, 115.9, 114.9, 112.9, 48.7, 42.8, 41.7, 36.0, 29.7, 26.7, 22.2, 14.2. HRMS (ESI) m/z calculated for C₃₁H₃₄N₆O₄, [M+H]⁺ 554.2642; found: 555.2714.

Compound 4 was synthesized as follows. To a solution of the imidazoquinoline³ (30 mg, 0.084 mmol) and 9 (20 mg, 0.093 mmol) in 5 mL DMF stirred at room temperature under argon was added triethylamine (0.015 mL, 1.5 equiv., 0.15 mmol) and 1-[Bis(dimethylamino)methylene]-1H-1,2,3-triazolo[4,5-b]pyridinium 3-oxide hexafluorophosphate (HATU) (41 mg, 1.1 equiv, 0.12 mmol in 0.2 mL DMF). The mixture was stirred at room temperature for 12 h. The reaction mixture was loaded on a silica column and purified by using column chromatography using DCM/MeOH 9:1 to yield an off-white powder. (30 mg, 55% yield) ¹H NMR (400 MHz, DMSO) δ 13.55 (s, 1H), 9.57 (s, 1H), 9.15 (t, J=5.9 Hz, 1H), 8.09 (s, 1H), 7.97 (d, J=8.3 Hz, 1H), 7.86-7.72 (m, 3H), 7.62 (t, J=7.7 Hz, 1H), 7.53 (t, J=7.7 Hz, 1H), 7.37 (t, J=7.7 Hz, 1H), 7.31 (d, J=8.4 Hz, 2H), 7.26 (d, J=7.9 Hz, 1H), 7.12 (d, J=7.8 Hz, 1H), 7.07 (dd, J=11.9, 5.1 Hz, 3H), 6.80 (dd, J=8.0, 2.1 Hz, 1H), 5.95 (s, 2H), 4.47 (d, J=5.7 Hz, 2H), 3.01-2.90 (m, 2H), 1.79-1.64 (m, 2H), 1.44-1.31 (m, 2H), 0.87 (t, J=7.3 Hz, 3H). ¹³C NMR (100 MHz, DMSO) δ 166.5, 158.7, 158.4, 157.8, 149.5, 141.4, 140.8, 139.7, 135.8, 135.2, 134.4, 130.4, 129.8, 129.4, 128.9, 126.8, 126.4, 125.7, 125.1, 122.2, 119.4, 118.3, 115.2, 114.1, 112.9, 48.7, 42.6, 35.5, 32.3, 29.7, 26.6, 22.2, 14.1. HRMS (ESI) m/z calculated for C₃₅H₃₃N₅O₂, [M+H]⁺ 555.2623; found: 556.2718.

Compound 5 was synthesized as follows. To a solution of the imidazoquinoline³ (30 mg, 0.083 mmol) and 10 (35 mg, 0.15 mmol) in 5 mL DMF stirred at room temperature under argon was added triethylamine (0.015 mL, 1.5 equiv., 0.15 mmol) and HATU (50 mg, 1.1 equiv, 0.13 mmol in 0.2 mL DMF). The mixture was stirred at room temperature for 12 h. The reaction mixture was loaded on a silica column and purified by using column chromatography using DCM/MeOH 9:1 to yield an off-white powder. The product was further purified using preparative HPLC (20 mg, 55% yield) ¹H NMR (400 MHz, DMSO) δ 12.53 (s, 1H), 9.57 (d, J=7.0 Hz, 1H), 9.33 (t, J=5.9 Hz, 2H), 8.02-7.85 (m, 4H), 7.78 (dd, J=11.6, 8.2 Hz, 3H), 7.58 (t, J=7.7 Hz, 2H), 7.29 (m, J=15.8, 7.8 Hz, 9H), 7.14-6.96 (m, 13H), 6.85-6.74 (m, 3H), 6.52 (s, 3H), 5.93 (s, 4H), 4.48 (d, J=5.8 Hz, 4H), 3.02-2.89 (m, 5H), 1.72 (m, J=15.3, 7.5 Hz, 5H), 1.36 (m, J=17.0, 8.5 Hz, 2H), 0.85 (dd, J=12.4, 5.0 Hz, 3H). ¹³C NMR (100 MHz, DMSO) δ 169.4, 161.0, 158.4, 157.4, 149.4, 145.9, 139.2, 135.8, 135.0, 134.6, 134.8, 129.9, 129.8, 128.6, 128.5, 128.6, 126.3, 126.1, 125.2, 125.1, 122.0, 118.9, 118.3, 116.8, 116.2, 115.4, 114.4, 113.4, 112.9, 48.7, 42.4, 29.7, 26.7, 22.2, 14.1 HRMS (ESI) m/z calculated for C₃₅H₃₃N₅O₃, [M+H]⁺ 571.2565; found: 572.2640.

Compound 6 was synthesized as follows. To a solution of the imidazoquinoline (ref) (30 mg, 0.084 mmol) and 11 (20 mg, 0.093 mmol) in 5 mL DMF stirred at room temperature under argon was added triethylamine (0.015 mL, 1.5 equiv., 0.15 mmol) and HATU (41 mg, 1.1 equiv, 0.12 mmol in 0.2 mL DMF). The mixture was stirred at room temperature for 12 h. The reaction mixture was loaded on a silica column and purified by using column chromatography using DCM/MeOH 9:1 to yield an off-white powder. The product was further purified using preparative HPLC (33.3 mg, 75% yield) ¹H NMR (400 MHz, DMSO) δ 13.48 (s, 1H), 12.48 (s, 1H), 9.46 (d, J=6.1 Hz, 2H), 8.07 (d, J=2.2 Hz, 2H), 7.96 (d, J=8.1 Hz, 2H), 7.78 (d, J=8.2 Hz, 2H), 7.62 (dd, J=12.5, 4.1 Hz, 2H), 7.44 (dd, J=11.3, 5.3 Hz, 3H), 7.35 (dd, J=14.0, 6.9 Hz, 3H), 7.31 (d, J=8.2 Hz, 2H), 7.05 (d, J=8.2 Hz, 2H), 6.91 (dd, J=10.1, 5.4 Hz, 4H), 6.84-6.77 (m, 9H), 5.94 (s, 2H), 4.49 (d, J=5.7 Hz, 2H), 3.00-2.89 (m, 2H), 1.71 (m, J=15.3, 7.5 Hz, 2H), 1.37 (m, J=14.6, 7.3 Hz, 2H), 0.90-0.78 (m, 3H). ¹³C NMR (100 MHz, DMSO) δ 169.1, 160.7, 158.3, 157.4, 149.4, 146.0, 140.7, 139.1, 135.8, 134.6, 134.8, 130.5, 129.9, 128.9, 128.4, 126.1, 125.2, 125.1, 122.0, 118.9, 118.3, 117.9, 117.6, 115.8, 115.8, 114.6, 113.9, 112.9, 48.7, 42.4, 29.7, 26.7, 22.2, 14.1 HRMS (ESI) m/z calculated for C₃₅H₃₃N₅O₃, [M+H]⁺ 571.2583; found: 572.2659.

Compound 7 was synthesized as follows. To a solution of trans-ferulic acid (200 mg, 1.03 mmol, 1.0 equiv.) and 3-azidopropylamine (0.15 mL, 1.5 mmol, 1.5 equiv.) in 5 mL DMF, was added triethylamine (0.14 mL, 1.02 mmol, 1.0 equiv.) and HATU (390 mg, 1.02 mmol, 1.0 equiv.) and the solution stirred for 12 h at RT under argon. The reaction mixture was extracted into ethyl acetate (10 mL×3) and the solvent evaporated in vacuo. The crude product obtained from the organic layer was dissolved in 5 mL MeOH/H₂O 4:1 and to this solution Tris(2-carboxyethyl) phosphine hydrochloride (295 mg, 1.03 mmol) was added and the mixture stirred for 12 h at RT. The reaction mixture was loaded on a silica column and purified by using column chromatography using DCM/MeOH 9:1 to yield an amorphous solid (80 mg, 67% yield). ¹H NMR (400 MHz, DMSO) δ 7.33 (d, J=15.7 Hz, 1H), 7.13 (d, J=1.7 Hz, 1H), 6.99 (dd, J=8.2, 1.7 Hz, 1H), 6.79 (d, J=8.1 Hz, 1H), 6.45 (d, J=15.7 Hz, 1H), 3.80 (s, 3H), 3.24 (q, J=6.5 Hz, 2H), 2.81 (m, J=12.4, 6.1 Hz, 2H), 1.78-1.68 (m, 2H). ¹³C NMR (100 MHz, DMSO) δ 166.4, 148.8, 148.3, 139.7, 126.7, 122.10, 119.0, 116.1, 111.2, 55.9, 37.3, 36.2, 28.0. HRMS (ESI) m/z calculated for C₁₃H₁₈N₂O₃, [M+Na]⁺ 250.1317; found: 274.1242.

Compound 8 was synthesized as follows. To a solution of 4-hydroxy-3-methoxybenzylamine hydrochloride (75 mg, 0.40 mmol, 1.0 equiv.) and 6-Azidohexanoic Acid NHS ester (100 mg, 0.40 mmol, 1.0 equiv.) in 1 mL DMF was added triethylamine (0.06 mL, 0.43 mmol, 1.2 equiv.) and the solution stirred for 12 h at RT under argon. The reaction mixture was dissolved in 5 mL MeOH/H₂O 4:1 and to this solution Tris(2-carboxyethyl) phosphine hydrochloride (120 mg, 0.40 mmol) was added and the mixture stirred for 12 h at RT. The reaction mixture was loaded on a silica column and purified by using column chromatography using DCM/MeOH 9:1 to yield an amorphous solid (58 mg, 50% yield). ¹H NMR (400 MHz, DMSO) δ 6.81 (d, J=1.7 Hz, 1H), 6.70 (d, J=8.0 Hz, 1H), 6.63 (dd, J=8.0, 1.8 Hz, 1H), 4.15 (d, J=5.8 Hz, 2H), 3.74 (s, 3H), 2.84-2.70 (m, 2H), 2.17-2.06 (m, 2H), 1.59-1.44 (m, 4H), 1.36-1.23 (m, 2H). ¹³C NMR (100 MHz, DMSO) δ 172.2, 147.9, 145.8, 130.9, 120.2, 115.6, 112.2, 56.0, 42.3, 35.6, 27.3, 25.9, 25.3. HRMS (ESI) m/z calculated for C₁₄H₂₂N₂O₃, [M+H]⁺ 266.163; found: 267.1705.

Compound 9 was synthesized as follows. A mixture of 3-hydroxyphenylboronic acid (220 mg, 1.59 mmol) 3-iodobenzoic acid (200 mg, 0.81 mmol) potassium carbonate (400 mg, 2.89 mmol) and Pd/C (10%) in 20 mL H₂O was refluxed at 80° C. for 4 h. Solution was acidified with 1M HCl and extracted with ethylacetate and washed with brine. Solvent evaporated in vacuo. Compound was purified by column chromatography to yield product as white powder (120 mg, 69% yield) ¹H NMR (400 MHz, DMSO) δ 8.13 (t, J=1.6 Hz, 1H), 7.96-7.91 (m, 1H), 7.88-7.82 (m, 1H), 7.58 (dd, J=9.6, 5.8 Hz, 1H), 7.28 (t, J=7.9 Hz, 1H), 7.13-7.05 (m, 2H), 6.81 (dd, J=7.8, 2.0 Hz, 1H). ¹³C NMR (100 MHz, DMSO) δ 167.7, 158.4, 141.1, 141.1, 131.9, 131.4, 130.6, 129.8, 128.6, 127.6, 117.9, 115.4, 113.9. HRMS (ESI) m/z calculated for C₁₃H₁₀O₃, [M+H]⁺ 214.063; found: 215.0661.

Compound 10 was synthesized as follows. A mixture of 3-hydroxyphenylboronic acid (220 mg, 1.59 mmol) 2-hydroxy-4-iodobenzoic acid (210 mg, 0.79 mmol) potassium carbonate (400 mg, 2.89 mmol) and Pd/C (10%) in 20 mL H₂O was refluxed at 80° C. for 4 h. Solution was acidified with 1M HCl and extracted with ethylacetate and washed with brine. Solvent evaporated in vacuo. Compound was purified by column chromatography to yield product as white powder (126 mg, 68%) ¹H NMR (400 MHz, DMSO) δ 7.28 (t, J=7.9 Hz, 1H), 7.15 (m, J=14.2, 10.4, 5.0 Hz, 3H), 7.05 (dd, J=5.2, 3.2 Hz, 1H), 6.86-6.80 (m, 1H). ¹³C NMR (100 MHz, DMSO) δ 172.2, 161.8, 158.3, 158.1, 147.8, 140.5, 131.3, 130.5, 118.1, 116.0, 115.0, 114.1, 112.3. HRMS (ESI) m/z calculated for C₁₃H₁₀O₄, [M+H]⁺ 230.0579; found: 231.0579.

For synthesis of Compound 11, a mixture of 4-hydroxyphenylboronic acid (220 mg, 1.59 mmol) 3-iodobenzoic acid (200 mg, 0.79 mmol) potassium carbonate (400 mg, 2.89 mmol) and Pd/C (10%) in 20 mL H₂O was refluxed at 80° C. for 4 h. Solution was acidified with 1M HCl and extracted with ethylacetate and washed with brine. Solvent evaporated in vacuo. Compound was purified by column chromatography to yield product as white powder (75 mg, 41% yield) ¹H NMR (400 MHz, DMSO) δ 7.93 (t, J=5.3 Hz, 1H), 7.74 (dd, J=8.6, 2.5 Hz, 1H), 7.43 (m, J=7.7, 4.8, 2.4 Hz, 3H), 7.01 (d, J=8.6 Hz, 1H), 6.86-6.81 (m, 3H). ¹³C NMR (100 MHz, DMSO) δ 172.3, 160.2, 157.2, 133.8, 132.0, 130.3, 127.7, 127.6, 127.5, 118.1, 116.2, 116.2, 113.6. HRMS (ESI) m/z calculated for C₁₃H₁₀O₄, [M+H]⁺ 230.0579; found: 231.0545.

Compound 12 was synthesized as follows. To a solution of imidazoquinoline (60 mg, 0.167 mmol) and triethylamine (0.023, 0.167 mmol) was added N,N′-Disuccinimidyl carbonate (60 mg, 0.235 mmol) was added. The reaction mixture was stirred at room temperature for 6 h. The reaction mixture was loaded on a silica column and purified by using column chromatography using DCM/MeOH 9:1 to yield a low melting clear solid (40 mg, 60% yield) 1H NMR (500 MHz, DMSO) δ 13.90 (s, 1H), 8.85 (t, J=6.0 Hz, 1H), 7.96 (d, J=8.3 Hz, 1H), 7.79 (d, J=8.3 Hz, 1H), 7.69-7.55 (m, 1H), 7.36 (dd, J=21.9, 14.0 Hz, 1H), 7.22 (dd, J=16.2, 8.0 Hz, 2H), 7.05 (dd, J=22.6, 7.6 Hz, 2H), 5.95 (s, 2H), 4.23 (d, J=3.6 Hz, 2H), 2.96 (t, J=7.7 Hz, 2H), 2.75 (s, 4H), 1.73 (m, J=15.2, 7.7 Hz, 2H), 1.38 (m, J=14.6, 7.4 Hz, 2H), 0.87 (dd, J=7.8, 6.9 Hz, 3H). 13C NMR (125 MHz, DMSO) δ 171.3, 157.5, 152.6, 148.7, 146.0, 138.3, 135.9, 134.9, 129.9, 128.2, 126.2, 125.2, 125.1, 122.5, 118.9, 112.8, 48.7, 44.4, 29.6, 26.6, 25.7, 22.2, 14.1. HRMS (ESI) m/z calculated for C₂₇H₂₈N₆O₄, [M+H]+; 500.2172 found: 501.2261.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and those cited elsewhere herein, to the extent that they provide exemplary procedural or other details supplementary to those set forth herein, are specifically incorporated herein by reference.
(1) Van Duin, D.; Medzhitov, R.; Shaw, A. C. Triggering TLR Signaling in Vaccination. Trends in Immunology. 2006. https://doi.org/10.1016/j.it.2005.11.005.
(2) Duthie, M. S.; Windish, H. P.; Fox, C. B.; Reed, S. G. Use of Defined TLR Ligands as Adjuvants within Human Vaccines. Immunol. Rev. 2011. https://doi.org/10.1111/j.1600-065X.2010.00978.x.
(3) Steinhagen, F.; Kinjo, T.; Bode, C.; Klinman, D. M. TLR-Based Immune Adjuvants. Vaccine. 2011. https://doi.org/10.1016/j.vaccine.2010.08.002.
(4) Dunne, A.; Marshall, N. A.; Mills, K. H. G. TLR Based Therapeutics. Current Opinion in Pharmacology. 2011. https://doi.org/10.1016/j.coph.2011.03.004.
(5) Mifsud, E. J.; Tan, A. C. L.; Jackson, D. C. TLR Agonists as Modulators of the Innate Immune Response and Their Potential as Agents against Infectious Disease. Frontiers in Immunology. 2014. https://doi.org/10.3389/fimmu.2014.00079.
(6) Mancini, R. J.; Stutts, L.; Ryu, K. A.; Tom, J. K.; Esser-Kahn, A. P. Directing the Immune System with Chemical Compounds. ACS Chem. Biol. 2014. https://doi.org/10.1021/cb500079s.
(7) Del Giudice, G.; Rappuoli, R.; Didierlaurent, A. M. Correlates of Adjuvanticity: A Review on Adjuvants in Licensed Vaccines. Seminars in Immunology. 2018. https://doi.org/10.1016/j.smim.2018.05.001.
(8) Flower, D. R. Systematic Identification of Small Molecule Adjuvants. Expert Opin. Drug Discov. 2012, 7 (9), 807-817. https://doi.org/10.1517/17460441.2012.699958.
(9) Wu, T. Y.-H.; Singh, M.; Miller, A. T.; Gregorio, E. De; Doro, F.; Oro, U. D.; Skibinski, D. a. G.; Mbow, M. L.; Bufali, S.; Herman, A. E.; Cortez, A.; Li, Y.; Nayak, B. P.; Tritto, E.; Filippi, C. M.; Otten, G. R.; Brito, L. a.; Monaci, E.; Li, C.; Aprea, S.; Valentini, S.; Calabr, S.; Laera, D.; Brunelli, B.; Caproni, E.; Malyala, P.; Panchal, R. G.; De Gregorio, E.; Doro, F.; D'Oro, U.; Skibinski, D. a. G.; Mbow, M. L.; Bufali, S.; Herman, A. E.; Cortez, A.; Li, Y.; Nayak, B. P.; Tritto, E.; Filippi, C. M.; Otten, G. R.; Brito, L. a.; Monaci, E.; Li, C.; Aprea, S.; Valentini, S.; Calabro, S.; Laera, D.; Brunelli, B.; Caproni, E.; Malyala, P.; Panchal, R. G.; Warren, T. K.; Bavari, S.; O'Hagan, D. T.; Cooke, M. P.; Valiante, N. M. Rational Design of Small Molecules as Vaccine Adjuvants. Sci. Transl. Med. 2014, 6 (263), 263ra160—263ra160. https://doi.org/10.1126/scitranslmed.3009980.
(10) Tyring, S.; Conant, M.; Marini, M.; Van Der Meijden, W.; Washenik, K. Imiquimod; An International Update on Therapeutic Uses in Dermatology. International Journal of Dermatology. 2002. https://doi.org/10.1046/j.1365-4362.2002.01597.x.
(11) Meyer, T.; Stockfleth, E. Clinical Investigations of Toll-like Receptor Agonists. Expert Opinion on Investigational Drugs. 2008. https://doi.org/10.1517/13543784.17.7.1051.
(12) Smith, A. J.; Li, Y.; Bazin, H. G.; St-Jean, J. R.; Larocque, D.; Evans, J. T.; Baldridge, J. R. Evaluation of Novel Synthetic TLR7/8 Agonists as Vaccine Adjuvants. Vaccine 2016. https://doi.org/10.1016/j.vaccine.2016.06.080.
(13) Ramakrishna, V.; Vasilakos, J. P.; Tario, J. D.; Berger, M. A.; Wallace, P. K.; Keler, T. Toll-like Receptor Activation Enhances Cell-Mediated Immunity Induced by an Antibody Vaccine Targeting Human Dendritic Cells. J. Transl. Med. 2007. https://doi.org/10.1186/1479-5876-5-5.
(14) Kim, W. G.; Choi, B.; Yang, H. J.; Han, J. A.; Jung, H.; Cho, H.; Kang, S.; Hong, S. Y. Covalent Conjugation of Small-Molecule Adjuvants to Nanoparticles Induces Robust Cytotoxic T Cell Responses via DC Activation. Bioconjug. Chem. 2016. https://doi.org/10.1021/acs.bioconjchem.6b00277.
(15) Kaczanowska, S.; Joseph, A. M.; Davila, E. TLR Agonists: Our Best Frenemy in Cancer Immunotherapy. J. Leukoc. Biol. 2013. https://doi.org/10.1189/jlb.1012501.
(16) Caron, G.; Duluc, D.; Fremaux, I.; Jeannin, P.; David, C.; Gascan, H.; Delneste, Y. Direct Stimulation of Human T Cells via TLR5 and TLR7/8: Flagellin and R-848 Up-Regulate Proliferation and IFN-γ Production by Memory CD4+ T Cells. J. Immunol. 2005. https://doi.org/10.4049/jimmunol.175.3.1551.
(17) Li, Q.; Yan, Y.; Liu, J.; Huang, X.; Zhang, X.; Kirschning, C.; Xu, H. C.; Lang, P. A.; Dittmer, U.; Zhang, E.; Lu, M. Toll-Like Receptor 7 Activation Enhances CD8+ T Cell Effector Functions by Promoting Cellular Glycolysis. Front. Immunol. 2019. https://doi.org/10.3389/fimmu.2019.02191.
(18) Dowling, D. J. Recent Advances in the Discovery and Delivery of TLR7/8 Agonists as Vaccine Adjuvants. ImmunoHorizons 2018. https://doi.org/10.4049/immunohorizons.1700063.
(19) Moser, B. A.; Escalante-buendia, Y.; Steinhardt, R. C.; Rosenberger, M. G.; Cassaidy, B. J.; Naorem, N.; Chon, A. C.; Nguyen, M.; Tran, N.; Aaron, P. Small Molecule NF-□B Inhibitors as Immune Potentiators for Enhancement of Vaccine Adjuvants Authors.
(20) Shukla, N. M.; Malladi, S. S.; Mutz, C. A.; Balakrishna, R.; David, S. A. Structure-Activity Relationships in Human Toll-like Receptor 7-Active Imidazoquinoline Analogues. J. Med. Chem. 2010. https://doi.org/10.1021/jm100358c.
(21) Murakami, Y.; Hirata, A.; Ito, S.; Shoji, M.; Tanaka, S.; Yasui, T.; Machino, M.; Fujisawa, S. Re-Evaluation of Cyclooxygenase-2-Inhibiting Activity of Vanillin and Guaiacol in Macrophages Stimulated with Lipopolysaccharide. Anticancer Res. 2007.
(22) Zheng, L. T.; Ryu, G. M.; Kwon, B. M.; Lee, W. H.; Suk, K. Anti-Inflammatory Effects of Catechols in Lipopolysaccharide-Stimulated Microglia Cells: Inhibition of Microglial Neurotoxicity. Eur. J. Pharmacol. 2008. https://doi.org/10.1016/j.ejphar.2008.04.035.
(23) Gilmore, T. D.; Herscovitch, M. Inhibitors of NF-KB Signaling: 785 and Counting. Oncogene. 2006. https://doi.org/10.1038/sj.onc.1209982.
(24) Elgueta, R.; Benson, M. J.; De Vries, V. C.; Wasiuk, A.; Guo, Y.; Noelle, R. J. Molecular Mechanism and Function of CD40/CD40L Engagement in the Immune System. Immunological Reviews. 2009. https://doi.org/10.1111/j.1600-065X.2009.00782.x.
(25) Mullins, S. R.; Vasilakos, J. P.; Deschler, K.; Grigsby, I.; Gillis, P.; John, J.; Elder, M. J.; Swales, J.; Timosenko, E.; Cooper, Z.; Dovedi, S. J.; Leishman, A. J.; Luheshi, N.; Elvecrog, J.; Tilahun, A.; Goodwin, R.; Herbst, R.; Tomai, M. A.; Wilkinson, R. W. Intratumoral Immunotherapy with TLR7/8 Agonist MEDI9197 Modulates the Tumor Microenvironment Leading to Enhanced Activity When Combined with Other Immunotherapies. J. Immunother. Cancer 2019. https://doi.org/10.1186/s40425-019-0724-8.

1. A compound of formula (I) 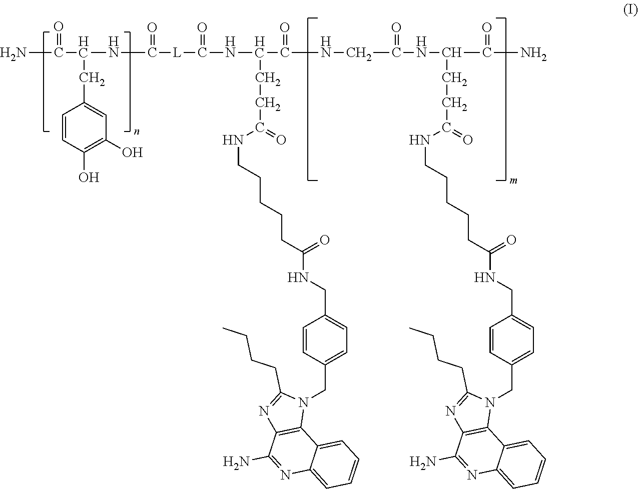
wherein L is a linker; and
wherein n is an integer from 1 to 10 and m is an integer from 0 to 10.

2. The compound of claim 1, wherein n is at least 2.

3. The compound of claim 1, wherein n is 5.

4. The compound of any of claims 1-3, wherein m is at least 1.

5. The compound of any of claims 1-3, wherein m is 4.

6. The compound of any of claims 1-5, wherein the linker is a bond, —NH—, —CH₂—, —C(O)—, —C(O)N—, -alkyl-C(O)—, -alkenyl-C(O)—, -alkyl-NC(O)—, -alkenyl-NC(O)—, —C(O)NC(O)—, —C(O)NH(CH₂)_{z}NC(O)—, -alkyl-C(O)NH(CH₂)_{z}NC(O)—, or -alkenyl-C(O)NH(CH₂)_{z}NC(O)—, where z is an integer from 1 to 10.

7. The compound of any of claims 1-5, wherein the linker comprises

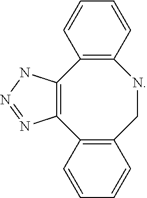

8. The compound of any of claims 1-7, wherein the compound is further defined as:

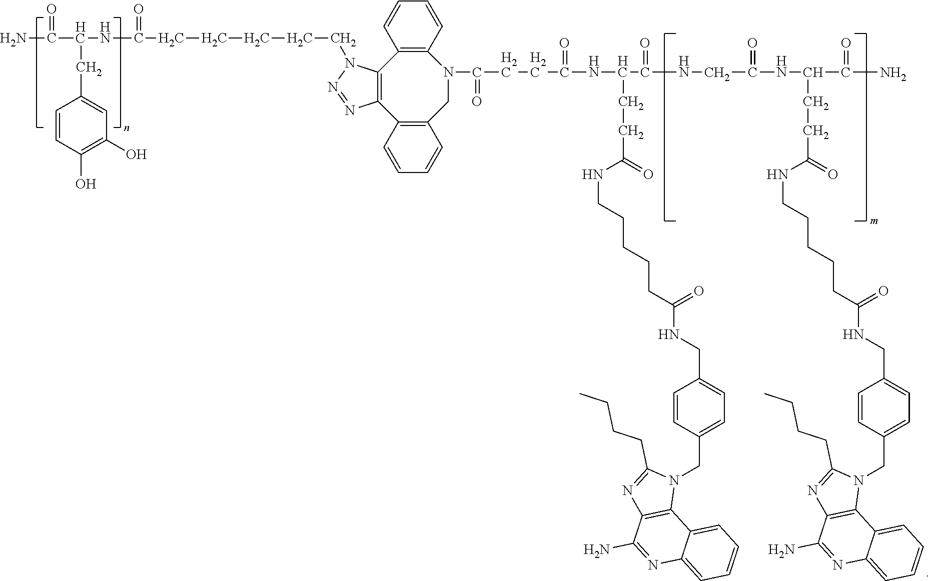

9. The compound of any of claims 1-8, wherein the compound is further defined as:

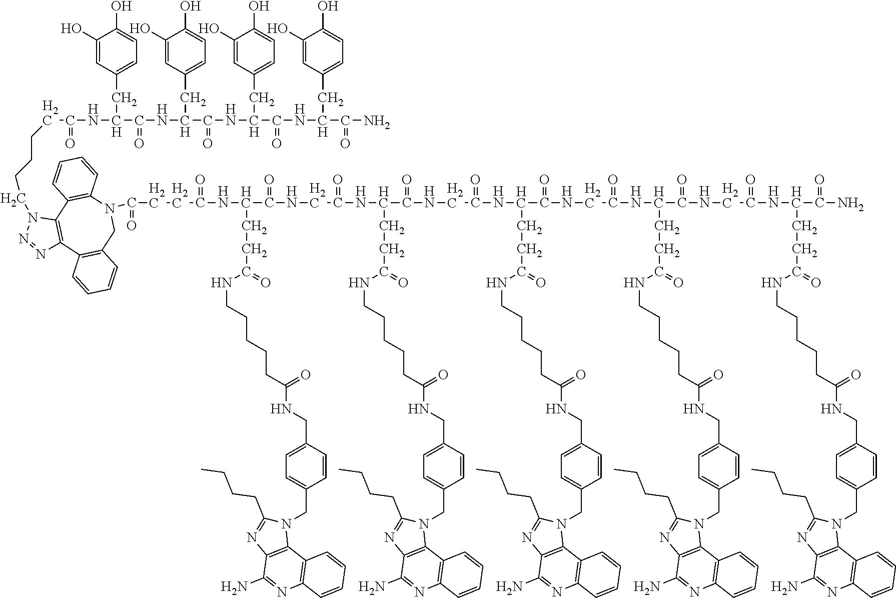

10. A compound of formula (II) 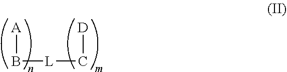
wherein:
A is an NF-κB inhibitor moiety;
B is an amino acid;
L is a linker;
C is an amino acid;
D is a TLR agonist moiety; and
wherein n is an integer from 1 to 10 and m is an integer from 1 to 10.

11. The compound of claim 10, wherein n is at least 2.

12. The compound of claim 10, wherein n is 5.

13. The compound of any of claims 10-12, wherein m is at least 2.

14. The compound of any of claims 10-12, wherein m is 5.

15. The compound of any of claims 10-15, wherein the TLR agonist moiety is a TLR7/8 agonist.

16. The compound of claim 15, wherein the TLR7/8 agonist is imidazoquinolinone or a derivative thereof.

17. The compound of any of claims 10-16, wherein the linker is a bond, —NH—, —CH₂—, —C(O)—, —C(O)N—, -alkyl-C(O)—, -alkenyl-C(O)—, -alkyl-NC(O)—, -alkenyl-NC(O)—, —C(O)NC(O)—, —C(O)NH(CH₂)_{z}NC(O)—, -alkyl-C(O)NH(CH₂)_{z}NC(O)—, or -alkenyl-C(O)NH(CH₂)_{z}NC(O)—, where z is an integer from 1 to 10.

18. The compound of any of claims 10-16, wherein the linker comprises

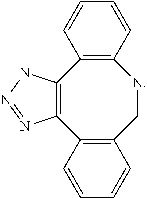

19. The compound of any of claims 10-18, wherein the NF-κB inhibitor moiety is ferrulic acid or a derivative thereof.

20. The compound of any of claims 10-18, wherein the NF-κB inhibitor moiety is vanillin or a derivative thereof.

21. The compound of any of claims 10-18, wherein the NF-κB inhibitor moiety is honokiol or a derivative.

22. The compound of any of claims 10-18, wherein the NF-κB inhibitor moiety is dopamine or a derivative thereof.

23. The compound of any of claims 10-22, wherein B is a glutamic acid.

24. The compound of any of claims 10-23, wherein C is a glycine.

25. The compound of any of claims 10-24, wherein the compound is further defined as: 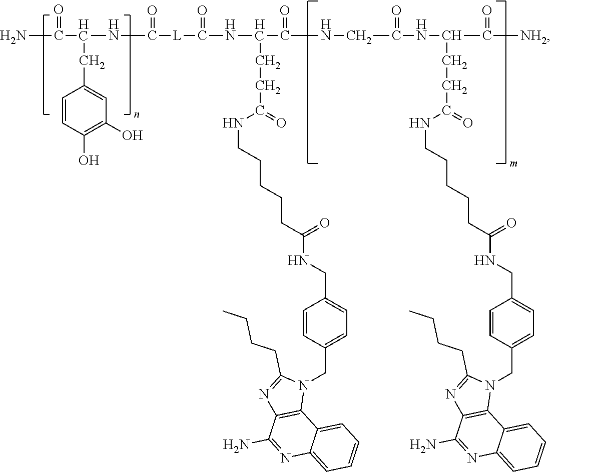
wherein L is a linker; and
wherein n is an integer from 1 to 10 and m is an integer from 0 to 10.

26. The compound of any of claims 10-24, wherein the compound is further defined as: 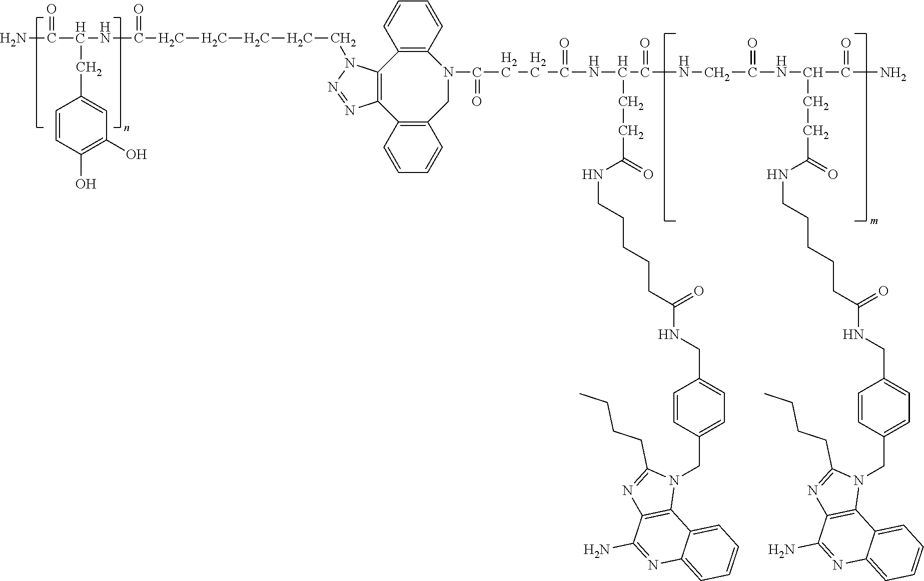
wherein n is an integer from 1 to 10 and m is an integer from 0 to 10.

27. The compound of any of claims 10-26, wherein the compound is further defined as:

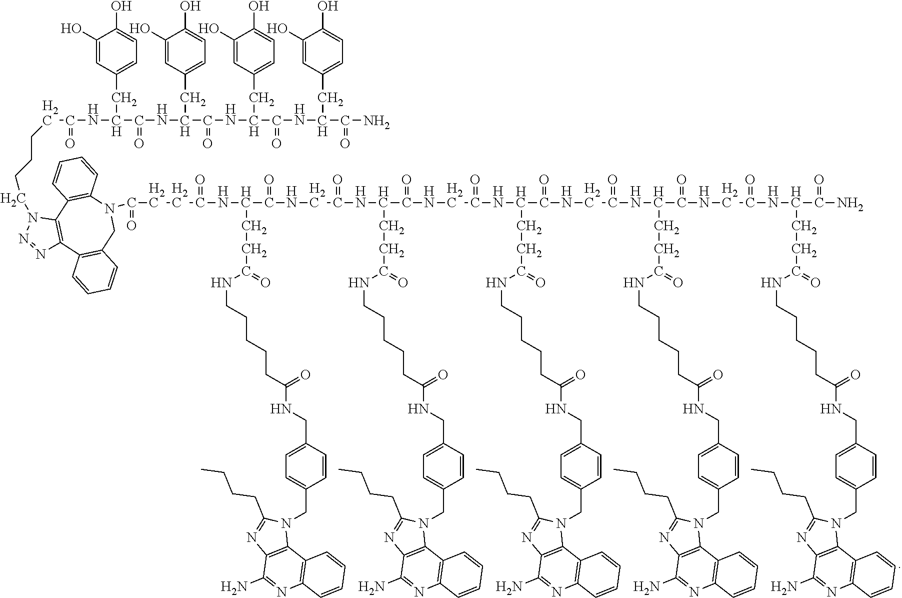

28. A compound having formula: 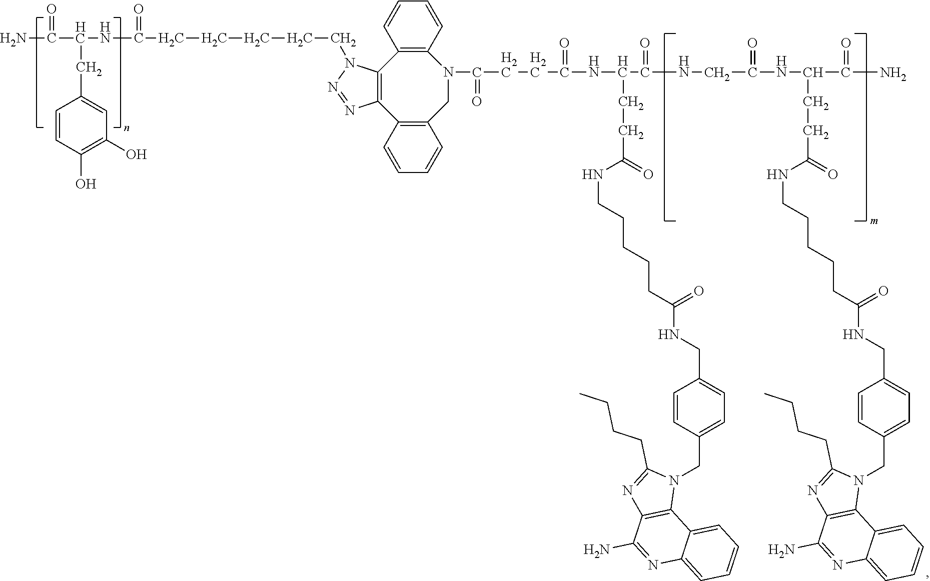
wherein n is an integer from 1 to 10 and m is an integer from 2 to 10.

29. The compound of claim 28, wherein the compound is further defined as:

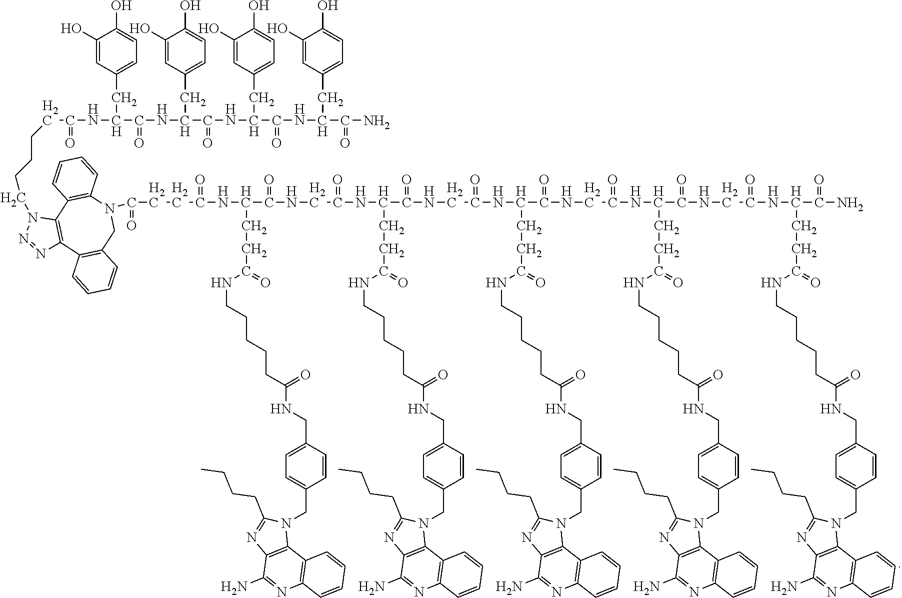

30. A method for immune activation comprising administering to a population of immune cells the compound of any of claims 1-29.

31. The method of claim 30, wherein the population of immune cells comprise T cells.

32. The method of claim 30, wherein the population of immune cells comprise macrophages.

33. The method of any of claims 30-32, wherein the method is an in vitro method.

34. The method of any of claims 30-32, wherein the method is an in vivo method.

35. A method for vaccinating a subject comprising administering to the subject an effective amount of a pharmaceutical composition comprising the compound of any of claims 1-27.

36. The method of claim 35, further comprising administering an antigen to the subject.

37. The method of claim 36, wherein the antigen is a bacterial antigen.

38. The method of claim 36, wherein the antigen is a viral antigen.

39. The method of claim 36, wherein the antigen is a dengue antigen.

40. The method of claim 39, wherein the dengue antigen is capsid protein of dengue serotype-2 (DENV-2C) or a portion thereof.

41. The method of claim 36, wherein the antigen is an HIV antigen.

42. The method of claim 41, wherein the HIV antigen is gp120 or a portion thereof.

43. The method of claim 36, wherein the antigen is a SARS-CoV-2 antigen.

44. The method of claim 43, wherein the SARS-CoV-2 antigen is a SARS-CoV-2 spike protein or a portion thereof.

45. The method of claim 36, wherein the antigen is a tumor antigen.

46. The method of any of claims 35-45, wherein the method is for preventing a disease in the subject.

47. The method of any of claims 35-45, wherein the method is for treating a disease in the subject.

48. The method of any of claims 35-47, wherein the subject has previously been administered an adjuvant.

49. The method of any of claims 35-47, wherein the subject has had an adverse reaction to the adjuvant.

50. A method for treatment or prevention of cancer, the method comprising administering to a subject an effective amount of a pharmaceutical composition comprising the compound of any of claims 1-24.

51. The method of claim 50, further comprising administering to the subject an additional cancer therapy.

52. The method of claim 51, wherein the additional cancer therapy comprises chemotherapy, radiation therapy, immunotherapy, or a combination thereof.

53. The method of claim 52, wherein the additional cancer therapy comprises immunotherapy.

54. The method of claim 53, wherein the additional cancer therapy is a checkpoint inhibitor therapy.

55. The method of any of claims 50-54, wherein the subject has not been diagnosed with cancer.

56. The method of any of claims 50-54, wherein the subject has been diagnosed with cancer.

57. The method of any of claims 50-56, wherein the subject was previously treated for cancer with a previous therapy.

58. The method of any of claims 50-56, wherein the subject was determined to be resistant to the previous therapy.

59. The method of any of claims 50-58, wherein the pharmaceutical composition is administered to the subject intratumorally.

60. A pharmaceutical composition comprising:
(a) the compound of any of claims 1-29; and
(b) an antigen.

61. The pharmaceutical composition of claim 60, wherein the antigen is a bacterial antigen

62. The pharmaceutical composition of claim 60, wherein the antigen is a viral antigen.

63. The pharmaceutical composition of claim 60, wherein the antigen is a dengue antigen.

64. The pharmaceutical composition of claim 63, wherein the dengue antigen is capsid protein of dengue serotype-2 (DENV-2C) or a portion thereof.

65. The pharmaceutical composition of claim 60, wherein the antigen is an HIV antigen.

66. The pharmaceutical composition of claim 65, wherein the HIV antigen is gp120 or a portion thereof.

67. The pharmaceutical composition of claim 60, wherein the antigen is a SARS-CoV-2 antigen.

68. The pharmaceutical composition of claim 67, wherein the SARS-CoV-2 antigen is a SARS-CoV-2 spike protein or a portion thereof.

69. The pharmaceutical composition of claim 60, wherein the antigen is a tumor antigen.

70. The pharmaceutical composition of any of claims 60-69, further comprising a pharmaceutically acceptable carrier.

71. A compound of formula (I): 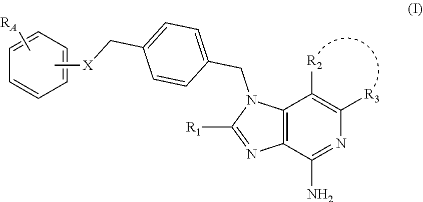
where
R₁ is H, C₁-C₁₂ alkyl, C₅-C₁₂ cycloalkyl, C₄-C₁₂ heterocycloalkyl, C₆-C₁₀ aryl, and C₄-C₁₀ heteroaryl, where R₁ is optionally substituted with one or more Y;
R₂ and R₃ are independently H, C₁-C₁₂ alkyl, C₂-C₁₂ alkenyl, C₂-C₁₂ alkynyl, C₁-C₁₂ heteroalkyl, C₂-C₁₂ heteroalkenyl, or C₂-C₁₂ heteroalkynyl, or together form a 5-6 membered carbocyclic or heterocyclic ring, where the 5-6 membered carbocyclic or heterocyclic ring is optionally substituted with one or more Y;
X is a bond, —N—, —C(O)—, -alkyl-C(O)—, -alkenyl-C(O)—, —C(O)N—, —NC(O)—, -alkyl-NC(O)—, alkenyl-NC(O)—, —C(O)N—, —C(O)NC(O)—, —C(O)NH(CH₂)_{z} NC(O)—, -alkyl-C(O)NH(CH₂)_{z} NC(O)—, or -alkenyl-C(O)NH(CH₂)_{z}NC(O)—, where z is an integer from 1 to 10;
R_{A} is attached to one or more ring atoms, and each R_{A} is independently hydrogen, hydroxyl, C₁-C₁₂ alkoxy, C₂-C₁₂ alkenoxy, C₂-C₁₂ alkenyl, or substituted or unsubstituted phenyl, and
Y is substituted alkyl, unsubstituted alkyl, substituted aryl, unsubstituted aryl, substituted heteroaryl, unsubstituted heteroaryl, substituted heterocycloalkyl, unsubstituted heterocycloalkyl, halogen, NH₂, NO₂, NR₄R₅, NC(O)R₅, OC(O)NR₄R₅, OC(O)OR₄, C(O)R₄, C(O)OR₄, SH, SR₄, OR₄, where R₄ and R₅ are independently C₁-C₆ substituted or unsubstituted alkyl.

72. The compound of claim 71, wherein R₁ is a C₁-C₁₂ alkyl group.

73. The compound of claim 72, wherein the alkyl group is an n-butyl group.

74. The compound of any of claims 71-73, wherein R₂ and R₃ together form a phenyl ring.

75. The compound of claim 74, wherein the phenyl ring is an unsubstituted phenyl ring.

76. The compound of any of claims 71-75, wherein X is an amide.

77. The compound of any of claims 71-75, wherein X comprises one or more of an alkyl group, an alkenyl group, an amide group, and a urea group.

78. The compound of any of claims 71-77, wherein R_{A} is unsubstituted phenyl.

79. The compound of any of claims 71-77, wherein R_{A} is substituted phenyl.

80. The compound of claim 71, wherein the compound is further defined as one of:

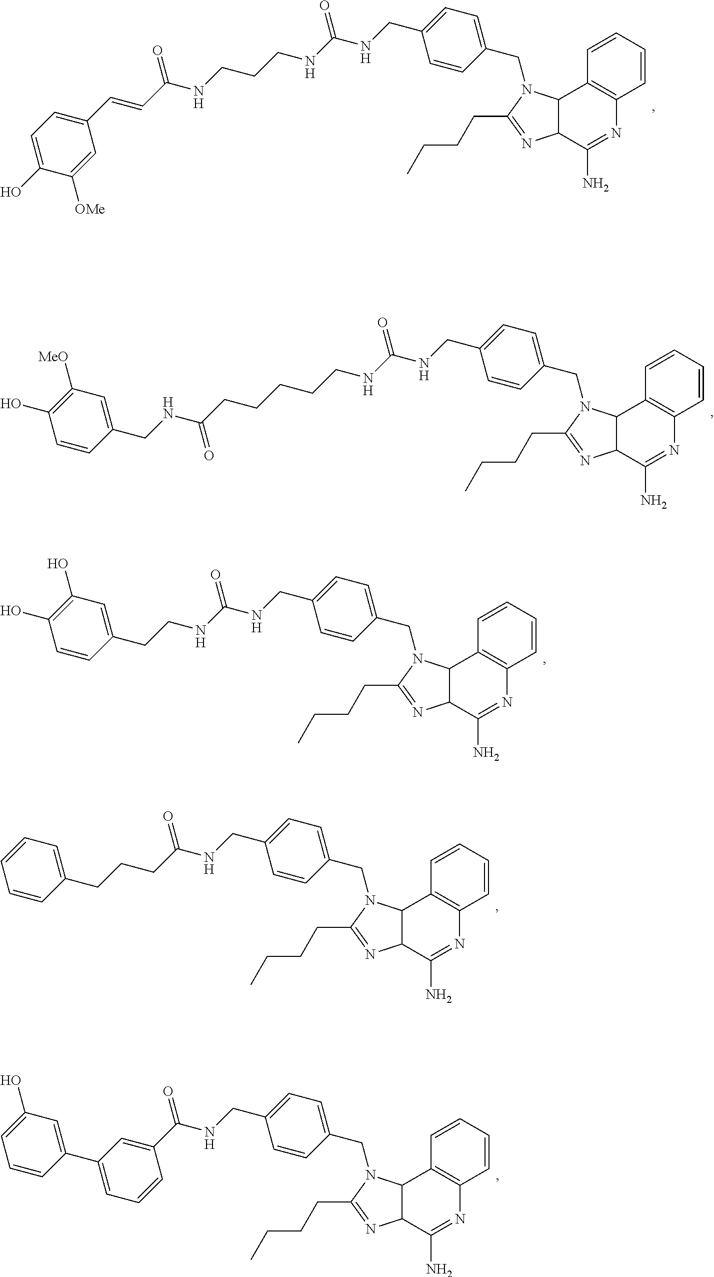 [SMILES: CCCCC1=NC2C(N)=Nc3ccccc3C2N1Cc1ccc(CNC(=O)NCCCNC(=O)/C=C/c2ccc(O)c(OC)c2)cc1 ; CCCCC1=NC2C(N)=Nc3ccccc3C2N1Cc1ccc(CNC(=O)NCCCCCC(=O)NCc2ccc(O)c(OC)c2)cc1 ; CCCCC1=NC2C(N)=Nc3ccccc3C2N1Cc1ccc(CNC(=O)NCCc2ccc(O)c(O)c2)cc1 ; CCCCC1=NC2C(N)=Nc3ccccc3C2N1Cc1ccc(CNC(=O)CCCc2ccccc2)cc1 ; CCCCC1=NC2C(N)=Nc3ccccc3C2N1Cc1ccc(CNC(=O)c2cccc(-c3cccc(O)c3)c2)cc1]

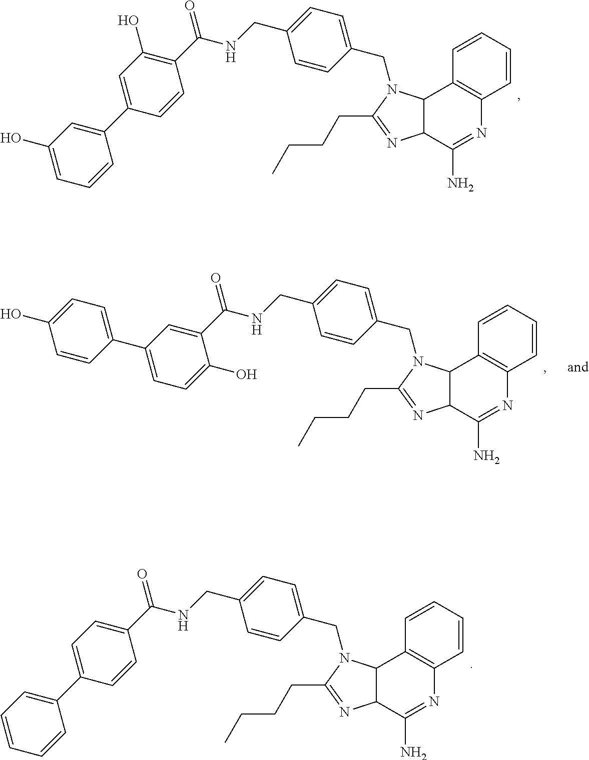 [SMILES: CCCCC1=NC2C(N)=Nc3ccccc3C2N1Cc1ccc(CNC(=O)c2ccc(-c3cccc(O)c3)cc2O)cc1 ; CCCCC1=NC2C(N)=Nc3ccccc3C2N1Cc1ccc(CNC(=O)c2cc(-c3ccc(O)cc3)ccc2O)cc1 ; CCCCC1=NC2C(N)=Nc3ccccc3C2N1Cc1ccc(CNC(=O)c2ccc(-c3ccccc3)cc2)cc1]

81. A compound of formula (II):
A-B-C (II)
wherein:
A is a TLR agonist;
B is a linker; and
C is an NF-κB inhibitor.

82. The compound of claim 81, wherein the TLR agonist is a TLR 7/8 agonist.

83. The compound of claim 82, wherein the TLR7/8 agonist is imidazoquinolinone or a derivative thereof.

84. The compound of any of claims 81-83, wherein the linker is a bond, —NH—, —CH₂—, —C(O)—, —C(O)N—, -alkyl-C(O)—, -alkenyl-C(O)—, -alkyl-NC(O)—, -alkenyl-NC(O)—, —C(O)NC(O)—, —C(O)NH(CH₂)_{z}NC(O)—, -alkyl-C(O)NH(CH₂)_{z}NC(O)—, or -alkenyl-C(O)NH(CH₂)_{z}NC(O)—, where z is an integer from 1 to 10.

85. The compound of any of claims 81-84, wherein the NF-κB inhibitor is ferrulic acid or a derivative thereof.

86. The compound of any of claims 81-84, wherein the NF-κB inhibitor is vanillin or a derivative thereof.

87. The compound of any of claims 81-84, wherein the NF-κB inhibitor is dopamine or a derivative thereof.

88. The compound of any of claims 81-84, wherein the NF-κB inhibitor is honokiol or a derivative thereof.

89. The compound of any of claims 81-88, wherein the compound is further defined as one of:

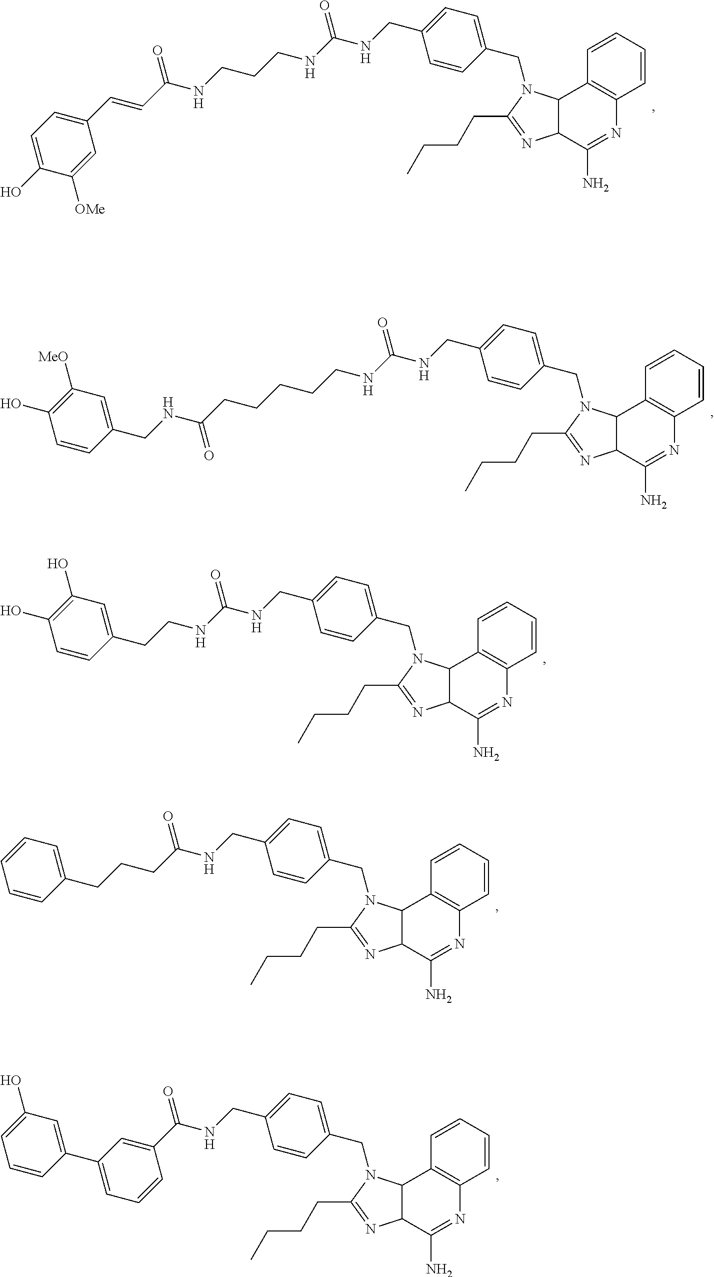 [SMILES: CCCCC1=NC2C(N)=Nc3ccccc3C2N1Cc1ccc(CNC(=O)NCCCNC(=O)/C=C/c2ccc(O)c(OC)c2)cc1 ; CCCCC1=NC2C(N)=Nc3ccccc3C2N1Cc1ccc(CNC(=O)NCCCCCC(=O)NCc2ccc(O)c(OC)c2)cc1 ; CCCCC1=NC2C(N)=Nc3ccccc3C2N1Cc1ccc(CNC(=O)NCCc2ccc(O)c(O)c2)cc1 ; CCCCC1=NC2C(N)=Nc3ccccc3C2N1Cc1ccc(CNC(=O)CCCc2ccccc2)cc1 ; CCCCC1=NC2C(N)=Nc3ccccc3C2N1Cc1ccc(CNC(=O)c2cccc(-c3cccc(O)c3)c2)cc1]

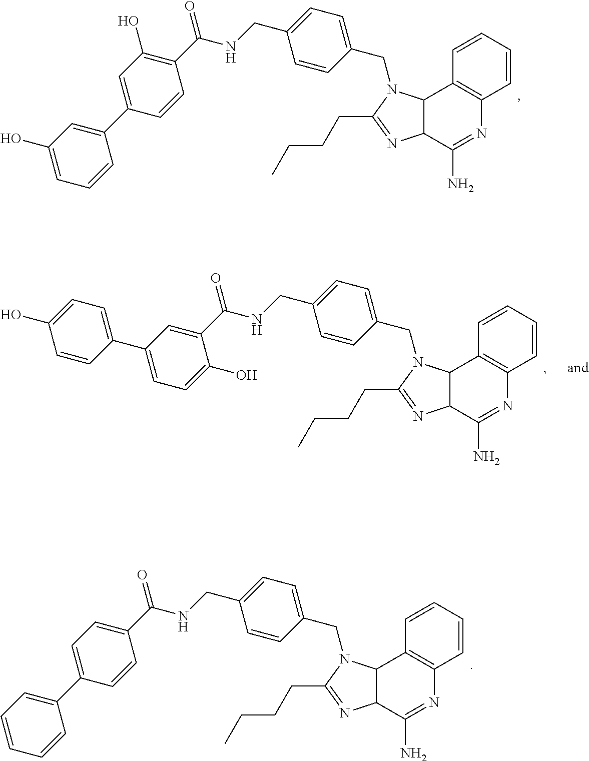 [SMILES: CCCCC1=NC2C(N)=Nc3ccccc3C2N1Cc1ccc(CNC(=O)c2ccc(-c3cccc(O)c3)cc2O)cc1 ; CCCCC1=NC2C(N)=Nc3ccccc3C2N1Cc1ccc(CNC(=O)c2cc(-c3ccc(O)cc3)ccc2O)cc1 ; CCCCC1=NC2C(N)=Nc3ccccc3C2N1Cc1ccc(CNC(=O)c2ccc(-c3ccccc3)cc2)cc1]

90. A method for vaccinating a subject comprising administering to the subject an effective amount of a pharmaceutical composition comprising (a) the compound of any of claims 71-80 or (b) the compound of any of claims 81-89.

91. The method of claim 90, further comprising administering an antigen to the subject.

92. The method of claim 91, wherein the antigen is a bacterial antigen.

93. The method of claim 91, wherein the antigen is a viral antigen.

94. The method of claim 91, wherein the antigen is a dengue antigen.

95. The method of claim 94, wherein the dengue antigen comprises capsid protein of dengue serotype-2 (DENV-2C).

96. The method of claim 91, wherein the antigen is an HIV antigen.

97. The method of claim 96, wherein the HIV antigen comprises gp120.

98. The method of claim 91, wherein the antigen is a SARS-CoV-2 antigen.

99. The method of claim 98, wherein the SARS-CoV-2 antigen is a SARS-CoV-2 spike protein or portion thereof.

100. The method of claim 91, wherein the antigen is a tumor antigen.

101. The method of any of claims 91-100, wherein the subject is a human subject.

102. The method of any of claims 91-101, wherein the method is for preventing a disease in the subject.

103. The method of any of claims 91-101, wherein the method is for treating a disease in the subject.

104. The method of any of claims 91-103, wherein the subject has previously been administered an adjuvant.

105. The method of claim 104, wherein the subject has had an adverse reaction to the adjuvant.

106. A method for treatment or prevention of cancer, the method comprising administering to a subject an effective amount of a pharmaceutical composition comprising (a) the compound of any of claims 71-80 or (b) the compound of any of claims 81-89.

107. The method of claim 106, further comprising administering to the subject an additional cancer therapy.

108. The method of claim 107, wherein the additional cancer therapy comprises chemotherapy, radiation therapy, immunotherapy, or a combination thereof.

109. The method of claim 107, wherein the additional cancer therapy comprises immunotherapy.

110. The method of claim 107, wherein the additional cancer therapy is a checkpoint inhibitor therapy.

111. The method of any of claims 106-110, wherein the subject is a human subject.

112. The method of any of claims 106-111, wherein the subject has not been diagnosed with cancer.

113. The method of any of claims 106-111, wherein the subject has been diagnosed with cancer.

114. The method of any of claims 106-113, wherein the subject was previously treated for cancer with a previous therapy.

115. The method of claim 114, wherein the subject was determined to be resistant to the previous therapy.

116. The method of any of claims 106-115, wherein the pharmaceutical composition is administered to the subject intratumorally.

117. A pharmaceutical composition comprising: the compound of any of claims 71-80 or the compound of any of claims 81-89; and an antigen.

118. The pharmaceutical composition of claim 117, wherein the antigen is a bacterial antigen.

119. The pharmaceutical composition of claim 117, wherein the antigen is a viral antigen.

120. The pharmaceutical composition of claim 117, wherein the antigen is a dengue antigen.

121. The pharmaceutical composition of claim 120, wherein the dengue antigen comprises capsid protein of dengue serotype-2 (DENV-2C).

122. The pharmaceutical composition of claim 117, wherein the antigen is an HIV antigen.

123. The pharmaceutical composition of claim 117, wherein the HIV antigen comprises gp120.

124. The pharmaceutical composition of claim 117, wherein the antigen is a SARS-CoV-2 antigen.

125. The pharmaceutical composition of claim 124, wherein the SARS-CoV-2 antigen is a SARS-CoV-2 spike protein or portion thereof.

126. The pharmaceutical composition of claim 117, wherein the antigen is an influenza antigen.

127. The pharmaceutical composition of claim 117, wherein the antigen is a tumor antigen.
